(19) United States
(12) Patent Application Publication
(10) Pub. No.: US 2026/0159902 A1
(43) Pub. Date: Jun. 11, 2026

(54) METHODS AND SYSTEMS FOR AMPLIFICATION AND DETECTION IN BIOLOGICAL SAMPLES

(71) Applicant: T2 Biosystems, Inc., Lexington, MA (US)

(72) Inventors: Mohamed Nabuan NAUFER, Malden, MA (US); Daniel Alejandro GAMERO, Chelmsford, MA (US); Jessica Lee SNYDER, Woburn, MA (US); Joseph MARTURANO, Bedford, MA (US); Mark DALTON, Lexington, MA (US); Christopher STEELE, Framingham, MA (US); Benjamin GALLO, Lexington, MA (US)

(21) Appl. No.: 18/706,871

(22) PCT Filed: Nov. 3, 2022
(86) PCT No.: PCT/US2022/079237

§ 371 Date: May 2, 2024

Related U.S. Application Data
(60) Provisional application No. 63/275,228, filed on Nov. 3, 2021.

(51) Int. Cl. C12Q 1/689; C12Q 1/6848

(52) U.S. Cl. CPC C12Q 1/689; C12Q 1/6848; C12Q 2600/16

(57) ABSTRACT

The invention features methods for amplification and detection of target nucleic acids in complex biological samples, for example, whole blood. The invention also features diagnostic and therapeutic methods based on amplification and detection of target nucleic acids characteristic of pathogens present in complex biological samples containing cells and/or cell debris, for example, whole blood.

### FIELD OF THE INVENTION

The invention features methods and systems for amplification and detection of target nucleic acids (e.g., DNA) in complex biological samples containing cells and/or cell debris as found in whole blood.

### BACKGROUND OF THE INVENTION

Whole blood contains interfering substances that can sometimes inhibit amplification methods (e.g., polymerase chain reaction (PCR)), which impedes direct detection of nucleic acid-based targets, whether from mammalian cells or from pathogens. This inhibition is especially problematic when specific loci must be amplified that are present only at minute concentrations, such as from one to ten microbial cells contained in a milliliter of human blood, which may be the case with pathogens that are present at low titer. For example, various heme compounds found in blood, including hemoglobin and hematin, have been shown to be inhibitory to Taq polymerase when added into PCR reactions. However, simply removing sources of heme compounds is not sufficient, as blood fractions lacking hemoglobin were also found to be inhibitory due to the presence of immunoglobulin G (IgG).

Another challenge in amplification of target nucleic acids from pathogens present in complex samples containing host cells and/or cell debris is presented by the enormous amount of mammalian DNA that is contained within the sample. For example, one milliliter of human blood contains approximately 3 to 6 million white blood cells. Since one human cell contains approximately 6 μg of nuclear DNA, 18 to 36 μg of human DNA is contained in one milliliter of crude blood lysate. In contrast, 10 bacterial cells contain 33 fg of DNA (based on a 2 Mbase genome). Thus, an approximate 8.4 billion-fold excess of human DNA over the microbial DNA of interest can exist. The inhibitory effects of high DNA concentrations in diagnostic assays aimed to detect pathogenic targets in total DNA extracted and purified from human blood is known in the art.

To reduce inhibition by interfering substances or high concentrations of non-target (e.g., host) nucleic acids, current assays for detecting pathogens in complex samples typically rely on nucleic acid isolation and fractionation/enrichment. Nucleic acid isolation is time-consuming, and loss of nucleic acids that are present in low copy numbers, such as microbial target DNA, may be lost during the process. Attempts at purifying the intact pathogen prior to nucleic acid isolation can also result in significant loss of target and reduced assay sensitivity. Another aspect of a lengthy purification process involving consumables and reagents is the danger of contamination with environmental and commensal microbial species. Therefore, minimal processing of complex samples before amplification and detection assays is desirable, and in some cases even necessary to achieve the highest levels of sensitivity and specificity.

Thus, there remains a need in the art for improved methods of amplifying and detecting target nucleic acids directly in complex samples containing cells and/or cell debris.

### SUMMARY OF THE INVENTION

The invention features methods for amplifying and detecting target nucleic acids in biological samples such as whole blood. The amplification (e.g., PCR) methods described herein are more sensitive than previously described amplification (e.g., PCR) methods, allowing for reliable detection of small number of pathogens in a blood sample. As a result, the amplification (e.g., PCR) methods described result in fewer false negative results and, accordingly, allow for more rapid and accurate diagnosis and treatment of a patient infected with a pathogen.

In one aspect, the invention features a method for amplifying and detecting a target pathogen nucleic acid in a biological sample suspected of containing one or more pathogen cells, the method comprising: (a) lysing one or more pathogen cells within the biological sample by contacting the biological sample with a lysis agent to form a solution containing both subject cell nucleic acid and pathogen nucleic acid, and optionally: (i) centrifuging the product of step (a) to form a supernatant and a pellet; (ii) discarding some or all of the supernatant of step (i) and optionally washing the pellet once; (iii) centrifuging the product of step (ii) to form a supernatant and a pellet; (iv) discarding some or all of the supernatant of step (iii) and mixing the pellet of step (iii) with a buffer; (b) amplifying the pathogen nucleic acid in the solution of step (a) by 6 to 20 cycles of PCR to form an amplified solution, wherein the PCR is symmetric PCR; (c) adding a pathogen-specific fluorescent probe to the amplified solution; (d) measuring a signal from the pathogen-specific fluorescent probe in the amplified solution of step (c) to obtain a baseline measurement; (e) further amplifying the pathogen nucleic acid in the amplified solution of step (c) by 30 to 50 cycles of PCR, wherein the PCR is asymmetric PCR; (f) measuring the signal from the pathogen-specific fluorescent probe in the amplified solution of step (e); and (g) obtaining a final signal measurement by subtracting the baseline measurement from step (d) from the signal from step (f), wherein the presence of the target pathogen in the biological sample is detected based on the final signal measurement.

In some aspects, the primer concentration for the symmetric PCR of step (b) is between 50 nM to 300 nM (e.g., 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, or 300 nM).

In some aspects, the excess primer concentration for the asymmetric PCR of step (e) is between 100 nM to 1000 nM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1000 nM).

In some aspects, the amplifying of step (b) is 8 cycles of PCR or 16 cycles of PCR.

In some aspects, the amplifying of step (e) is 46 cycles of PCR or 38 cycles of PCR.

In some aspects, the biological sample is a whole blood sample.

In some aspects, the lysis agent of step (a) is an erythrocyte lysis agent.

In some aspects, the amplifying of step (b) and step (e) is in a multiplexed PCR reaction configured to amplify a plurality of target pathogen nucleic acids.

In some aspects, step (c) comprises adding a plurality of different pathogen-specific fluorescent probes to the amplified solution.

In some aspects, the plurality of pathogen-specific fluorescent probes comprises three or more different pathogen-specific fluorescent probes.

In some aspects, the plurality of pathogen-specific fluorescent probes is between 5 and 15 probes.

In some aspects, a plurality (e.g., 2, 3, 4, 5, 6, or more) of pathogen-specific fluorescent probes is used to detect a single target nucleic acid. In some embodiments, 3 pathogen-specific fluorescent probes is used to detect a single target nucleic acid.

In some aspects, step (c) comprises transferring all or part of the amplified solution of step (b) to a multi-well plate containing the pathogen-specific fluorescent probe.

In some aspects, the multi-well plate is a 4-well plate, a 96-well plate, or a 384-well plate.

In some aspects, the multi-well plate is a 4-well plate.

In some aspects, the multi-well plate is a clear plate.

In some aspects, the multi-well plate is a black plate.

In some aspects, the pathogen-specific fluorescent probe of step (c) is present in a liquid volume or is a lyophilized fluorescent probe.

In some aspects, the pathogen-specific fluorescent probe of step (f) is present in a liquid volume.

In some aspects, the wells of the multi-well plate each comprise a reference dye.

In some aspects, the wells of the multi-well plate each comprise the same reference dye.

In some aspects, the wells of the multi-well plate each comprise different reference dyes.

In some aspects, the pathogen-specific fluorescent probe is a molecular beacon.

In some aspects, the molecular beacon comprises an organic dye fluorophore.

In some aspects, the organic dye fluorophore is ATTO 425, FAM, HEX, Cy5, ROX, ATTO 633, TAMARA, Cy5.5, or ALEXA® 750.

In some aspects, the organic dye fluorophore is ATTO 425, FAM, HEX, ROX, ATTO 633, Cy5.5, or ALEXA® 750.

In some aspects, the molecular beacon comprises a quantum dot.

In some aspects, the molecular beacon comprises an organic dye quencher.

In some aspects, the organic dye quencher is Black Hole Quencher 1, Black Hole Quencher 2, Iowa Black FQ, or Iowa Black RQ.

In some aspects, the molecular beacon comprises a gold nanoparticle or silica nanoparticle quencher.

In some aspects, an oil is added to a well of the multi-well plate after the amplified solution of step (b) is added.

In some aspects, the oil is a mineral oil.

In some aspects, the oil is a silicone oil.

In some aspects, the multi-well plate is sealed with a plastic seal.

In some aspects, the plastic seal covers the entire multi-well plate.

In some aspects, the plastic seal is a slit-seal.

In some aspects, the method comprises steps (i)-(iv), and wherein the pellet of step (ii) is washed by mixing with Tris-EDTA (TE) buffer.

In some aspects, the TE buffer has a volume of about 400 μL to about 2400 μL (e.g., about 400 μL, about 500 μL, about 600 μL, about 700 μL, about 800 μL, about 900 μL, about 1000 μL, about 1100 μL, about 1200 μL, about 1300 μL, about 1400 μL, about 1500 μL, about 1600 μL, about 1700 μL, about 1800 μL, about 1900 μL, about 2000 μL, about 2100 μL, about 2200 μL, about 2300 μL, or about 2400 UL).

In some aspects, the TE buffer has a volume of about 1200 μL.

In some aspects, the buffer of step (iv) is a PCR buffer that has a moderately alkaline pH at ambient temperature.

In some aspects, the lysing of step (a) is by detergent lysis, hypotonic lysis, and/or beadbeating.

In some aspects, the amplified solution of step (b) comprises whole blood proteins and non-target oligonucleotides.

In some aspects, the whole blood sample is from about 0.05 to about 10.0 mL.

In some aspects, the whole blood sample is (i) between about 1.25 and about 2.5 mL; or (ii) about 6 mL.

In some aspects, the whole blood sample is about 6 mL.

In another aspect, the invention features a method for amplifying and detecting a target pathogen nucleic acid in a biological sample, the method comprising: (a) contacting a biological sample suspected of containing one or more pathogen cells with a lysis agent, thereby lysing cells within the biological sample; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer to form a solution containing both subject cell nucleic acid and pathogen nucleic acid; (f) amplifying the pathogen nucleic acid in the solution of step (e) by 6 to 20 cycles of polymerase chain reaction (PCR) to form an amplified solution; (g) adding a pathogen-specific fluorescent probe to the amplified solution; (h) measuring a signal from the pathogen-specific fluorescent probe in the amplified solution of step (g) to obtain a baseline measurement; (i) further amplifying the pathogen nucleic acid in the amplified solution of step (g) by 30 to 50 cycles of PCR; (j) measuring the signal from the pathogen-specific fluorescent probe in the amplified solution of step (i); and (k) obtaining a final signal measurement by subtracting the baseline measurement from step (h) from the signal from step (j), wherein the presence of the target pathogen in the biological sample is detected based on the final signal measurement.

In some aspects, the amplifying of step (f) is symmetric PCR or asymmetric PCR.

In some aspects, the amplifying of step (f) is symmetric PCR.

In some aspects, the primer concentration for the symmetric PCR is between 50 nM to 300 nM (e.g., 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, or 300 nM).

In some aspects, the amplifying of step (f) is asymmetric PCR.

In some aspects, the amplifying of step (i) is asymmetric PCR.

In some aspects, the excess primer concentration for the asymmetric PCR is between 100 nM to 1000 nM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1000 nM).

In some aspects, the amplifying of step (f) is 8 cycles of PCR or 16 cycles of PCR.

In some aspects, the amplifying of step (i) is 46 cycles of PCR or 38 cycles of PCR.

In some aspects, the biological sample is a whole blood sample.

In some aspects, the lysis agent of step (a) is an erythrocyte lysis agent.

In some aspects, the amplifying of step (f) and step (i) is in a multiplexed PCR reaction configured to amplify a plurality of target pathogen nucleic acids.

In some aspects, step (g) comprises adding a plurality of different pathogen-specific fluorescent probes to the amplified solution.

In some aspects, the plurality of pathogen-specific fluorescent probes comprises three or more different pathogen-specific fluorescent probes.

In some aspects, the plurality of pathogen-specific fluorescent probes is between 5 and 15 probes.

In some aspects, a plurality (e.g., 2, 3, 4, 5, 6, or more) of pathogen-specific fluorescent probes is used to detect a single target nucleic acid. In some embodiments, 3 pathogen-specific fluorescent probes is used to detect a single target nucleic acid.

In some aspects, step (g) comprises transferring all or part of the amplified solution of step (f) to a multi-well plate containing the pathogen-specific fluorescent probe.

In some aspects, the multi-well plate is a 4-well plate, a 96-well plate, or a 384-well plate.

In some aspects, the multi-well plate is a 4-well plate.

In some aspects, the multi-well plate is a clear plate.

In some aspects, the multi-well plate is a black plate.

In some aspects, the pathogen-specific fluorescent probe of step (f) is present in a liquid volume or is a lyophilized fluorescent probe.

In some aspects, the pathogen-specific fluorescent probe of step (f) is present in a liquid volume.

In some aspects, the wells of the multi-well plate each comprise a reference dye.

In some aspects, the wells of the multi-well plate each comprise the same reference dye.

In some aspects, the wells of the multi-well plate each comprise different reference dyes.

In some aspects, the pathogen-specific fluorescent probe is a molecular beacon.

In some aspects, the molecular beacon comprises an organic dye fluorophore.

In some aspects, the organic dye fluorophore is ATTO 425, FAM, HEX, Cy5, ROX, ATTO 633, TAMARA, Cy5.5, or ALEXA® 750.

In some aspects, the organic dye fluorophore is ATTO 425, FAM, HEX, ROX, ATTO 633, Cy5.5, or ALEXA® 750.

In some aspects, the molecular beacon comprises a quantum dot.

In some aspects, the molecular beacon comprises an organic dye quencher.

In some aspects, the organic dye quencher is Black Hole Quencher 1, Black Hole Quencher 2, Iowa Black FQ, or Iowa Black RQ.

In some aspects, the molecular beacon comprises a gold nanoparticle or silica nanoparticle quencher.

In some aspects, an oil is added to a well of the multi-well plate after the amplified solution of step (f) is added.

In some aspects, the oil is a mineral oil.

In some aspects, the oil is a silicone oil.

In some aspects, the multi-well plate is sealed with a plastic seal.

In some aspects, the plastic seal covers the entire multi-well plate.

In some aspects, the plastic seal is a slit-seal.

In some aspects, the pellet of step (c) is washed by mixing with Tris-EDTA (TE) buffer.

In some aspects, the TE buffer has a volume of about 400 μL to about 2400 μL (e.g., about 400 μL, about 500 μL, about 600 μL, about 700 μL, about 800 μL, about 900 μL, about 1000 μL, about 1100 μL, about 1200 μL, about 1300 μL, about 1400 μL, about 1500 μL, about 1600 μL, about 1700 μL, about 1800 μL, about 1900 μL, about 2000 μL, about 2100 μL, about 2200 μL, about 2300 μL, or about 2400 UL).

In some aspects, the TE buffer has a volume of about 1200 μL.

In some aspects, the buffer of step (e) is a PCR buffer that has a moderately alkaline pH at ambient temperature.

In some aspects, the lysing step (a) is by detergent lysis, hypotonic lysis, and/or beadbeating.

In some aspects, the amplified solution of step (f) comprises whole blood proteins and non-target oligonucleotides.

In some aspects, the whole blood sample is from 0.05 to 10.0 mL.

In some aspects, the whole blood sample is (i) between about 1.25 and about 2.5 mL; or (ii) about 6 mL.

In some aspects, the whole blood sample is about 6 mL.

In some aspects, the pathogen is a Candida species.

In some aspects, the Candida species is selected from the group consisting of Candida albicans, Candida krusei, Candida glabrata, Candida parapsilosis, Candida auris, Candida lusitaniae, Candida dubliniensis, Candida kefyr, and Candida tropicalis.

In some aspects, the amplifying of steps (b) and (e) comprise amplifying a Candida nucleic acid to be detected in the presence of a forward primer and a reverse primer, each of which is universal to multiple Candida species to form a solution comprising a Candida amplicon.

In some aspects, the amplifying of steps (f) and (i) comprise amplifying a Candida nucleic acid to be detected in the presence of a forward primer and a reverse primer, each of which is universal to multiple Candida species to form a solution comprising a Candida amplicon.

In some aspects, the pathogen is a bacterial pathogen.

In some aspects, the bacterial pathogen is selected from the group consisting of Bacteroides fragilis, Burkholderia cepacia, Campylobacter jejuni/coli, Clostridium perfringens, Klebsiella aerogenes, Enterobacter cloacae, Enterococcus faecalis, Enterococcus faecium, Escherichia coli, Haemophilus influenzae, Kingella kingae, Klebsiella oxytoca, Klebsiella pneumoniae, Listeria monocytogenes, Morganella morganii, Neisseria meningitidis, Prevotella buccae, Prevotella intermedia, Prevotella melaninogenica, Propionibacterium acnes, Proteus mirabilis, Proteus vulgaris, Pseudomonas aeruginosa, Salmonella enterica, Serratia marcescens, Staphylococcus aureus, Staphylococcus haemolyticus, Staphylococcus lugdunensis, Stenotrophomonas maltophilia, Staphylococcus saprophyticus, Streptococcus agalactiae, Streptococcus bovis, Streptococcus dysgalactiae, Streptococcus mitis, Streptococcus mutans, Streptococcus pneumoniae, Streptococcus pyogenes, and Streptococcus sanguinis.

In some aspects, the bacterium is selected from the group consisting of Enterococcus faecalis, Enterococcus faecium, Staphylococcus aureus, Klebsiella pneumoniae, Acinetobacter baumannii, and Pseudomonas aeruginosa.

In some aspects, the bacterial pathogen is Escherichia coli.

In some aspects, the bacterium is selected from one or more of the group consisting of Escherichia coli, Enterococcus faecium, Staphylococcus aureus, Klebsiella pneumoniae, Acinetobacter baumannii and Pseudomonas aeruginosa.

In some aspects, the Staphylococcus aureus is methicillin-resistant Staphylococcus aureus (MRSA).

In some aspects, the bacterial pathogen is a Borrelia species.

In some aspects, the Borrelia species is Borrelia burgdorferi, Borrelia afzelii, or Borrelia garinii.

In some aspects, the target pathogen nucleic acid is characteristic of a genus of bacterial pathogens.

In some aspects, the genus of bacterial pathogens comprises coagulase negative staphylococci (CoNS), Enterobacter cloacae complex, Acinetobacter baumannii-calcoaceticus complex, Enterobacterales, Streptococcus spp., Citrobacter spp., Proteus spp., Serratia spp., Clostridium spp., Bacteroides spp., Pan Gram negative, or pan Gram positive.

In some aspects, the target pathogen nucleic acid is an antibiotic resistance target nucleic acid.

In some aspects, the antibiotic resistance target nucleic acid is mecA, mecC, KPC, NDM, VIM, IMP, OXA-48-like, CTX-M, CMY, DHA, FOX, mcr-1, OXA-23-like, or van A/vanB.

In some aspects, the pathogen is a viral pathogen.

In some aspects, the viral pathogen is a Cytomegalovirus (CMV), an Epstein Barr Virus, a BK Virus, a Hepatitis B virus, a Hepatitis C virus, a Herpes simplex virus (HSV), HSV1, HSV2, a Respiratory syncytial virus (RSV), an Influenza virus, Influenza A virus, Influenza A subtype H1 virus, Influenza A subtype H3 virus, Influenza B virus, Human Herpes Virus 6, Human Herpes Virus 8, Human Metapneumovirus (hMPV), a Rhinovirus, Parainfluenza 1 virus, Parainfluenza 2 virus, Parainfluenza 3 virus, an Adenovirus, or a Coronavirus.

In some aspects, the method comprises detecting the panel set forth in Table 3.

In some aspects, the method further comprises selecting a therapy for the patient based on the detection of the target pathogen nucleic acid.

In some aspects, the method further comprises the therapy to the patient based on the detection of the target pathogen nucleic acid.

Other features and advantages of the invention will be apparent from the following detailed description, drawings, and the claims.

### BRIEF DESCRIPTION OF THE DRAWINGS

FIG. 1 is a schematic diagram of an exemplary asymmetric PCR method and fluorescence detection approach of the present disclosure.

FIG. 2 is a schematic diagram of an exemplary symmetric-asymmetric PCR method and fluorescence detection approach of the present disclosure.

FIG. 3 is a graph showing fluorescence detection of vanA/vanB with asymmetric and symmetric-asymmetric methods.

FIG. 4 is a graph showing fluorescence detection of Serratia spp. with asymmetric and symmetric-asymmetric methods.

FIG. 5 is a graph showing fluorescence detection of E. faecium with asymmetric and symmetric-asymmetric methods.

FIG. 6 is a graph showing fluorescence detection of S. pneumoniae with asymmetric and symmetric-asymmetric methods.

FIG. 7 is a graph showing results of a limit of detection screen for vanA/vanB antibiotic resistance gene.

FIG. 8 is a graph showing results of a limit of detection screen for Serratia spp. channel.

FIG. 9 is a graph showing results of a limit of detection screen for E. faecium channel.

FIG. 10 is a graph showing results of a limit of detection screen for S. pneumoniae channel.

FIG. 11 is a graph showing results of a cross-reactivity assessment for the isolate of E. coli carrying CMY.

FIG. 12 is a graph showing results of a cross-reactivity assessment for the isolate K. pneumoniae carrying OXA-48 and CTX-M.

FIG. 13 is a graph showing results of a cross-reactivity assessment for isolate K. pneumoniae carrying VIM.

FIG. 14 is a graph showing results of a CMY single-versus multi-spike comparison.

FIG. 15 is a graph showing results of an OXA-48 single-versus multi-spike comparison.

FIG. 16 is a graph showing results of a VIM single-versus multi-spike comparison.

FIG. 17 is a graph showing results of a competitive inhibition study with E. faecium (Efm) and Serratia marcescens (Sm).

FIG. 18 is a series of graphs showing a comparison of the 2-stage PCR (deltaF) versus 1-stage PCR approach for MRN healthy donors for CMY-HEX.

FIG. 19 is a series of graphs showing a comparison of the 2-stage PCR (deltaF) versus 1-stage PCR approach for MRN healthy donors for mecA-Cy5.

FIG. 20 is a series of graphs showing a comparison of the 2-stage PCR (deltaF) versus 1-stage PCR approach for unhealthy patients for CMY-HEX.

FIG. 21 is a series of graphs showing a comparison of the 2-stage PCR (deltaF) versus 1-stage PCR approach for unhealthy patients for mecA-Cy5.

FIG. 22 is a series of graphs showing a comparison of the symmetric to asymmetric 2-stage PCR approach compared to the asymmetric 2-stage PCR approach. Individual values are represented by dots enclosed in a kernel density estimation that suggests probability based on the data distribution. The symmetric to asymmetric PCR condition produced delta F values significantly greater than the purely asymmetric PCR condition.

FIG. 23 is a graph showing a comparison of 1-stage and 2-stage symmetric-asymmetric amplification methods with 10 CFU/mL S. lugdunensis.

FIG. 24 is a graph showing a comparison of 1-stage and 2-stage symmetric-asymmetric amplification methods with 10 CFU/mL B. fragilis.

### DETAILED DESCRIPTION OF EMBODIMENTS OF THE INVENTION

The invention provides methods for amplification (e.g., multiplex amplification) and detection of one or more target nucleic acids in complex biological samples containing cells and/or cell debris. The methods described herein are more sensitive than previously described PCR methods, allowing for reliable detection of a small number of pathogens in a blood sample. As a result, the methods described result in fewer false negative results and, accordingly, allow for more rapid and accurate diagnosis and treatment of a patient infected with a pathogen. The amplification methods disclosed herein are also amenable to multiplexed reactions, allowing for sensitive detection of a large number of different target nucleic acids (e.g., panels of target nucleic acids disclosed herein). Further, the amplification methods disclosed herein also enable normalization that can reduce patient-to-patient variability, for example, by allowing for subtraction of aberrant autofluorescence present in patient samples following a first stage of PCR.

In some embodiments, detection of the target nucleic acid amplicon(s) allows for rapid, accurate, and high sensitivity detection and identification of a microbial or viral pathogen present in a biological sample containing host cells and/or cell debris (e.g., whole blood, processed whole blood (e.g., a crude whole blood lysate), serum, plasma, or other blood derivatives; bloody fluids such as wound exudate, phlegm, bile, and the like; tissue samples (e.g., tissue biopsies); and sputum (e.g., purulent sputum and bloody sputum)), which may be used, for example, for diagnosis of a disease (e.g., sepsis, bloodstream infections (BSIs) (e.g., bacteremia, fungemia (e.g., Candidemia), and viremia), Lyme disease, septic shock, and diseases that may manifest with similar symptoms to diseases caused by or associated with microbial pathogens, e.g., systemic inflammatory response syndrome (SIRS)). In some embodiments, the methods of the invention allow for amplification of target nucleic acids using nucleic acid polymerases (e.g., thermostable DNA polymerases, including commercially available thermostable DNA polymerases such as Taq) that are typically inhibited by the presence of complex samples containing cells and/or cell debris, e.g., blood.

### Definitions

The terms “amplification” or “amplify” or derivatives thereof as used herein mean one or more methods known in the art for copying a target or template nucleic acid, thereby increasing the number of copies of a selected nucleic acid sequence. Amplification may be exponential or linear. A target or template nucleic acid may be either DNA or RNA. The sequences amplified in this manner form an “amplified region” or “amplicon.” Primers and probes can be readily designed by those skilled in the art to target a specific template nucleic acid sequence. In some embodiments, amplification is performed by polymerase chain reaction (PCR). In some embodiments, the amplification is multiplex amplification (e.g., multiplexed for detection of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more target nucleic acids). In some embodiments, the multiplex amplification is configured to detect 12 target nucleic acids. In some embodiments, multiple multiplex amplification reactions are performed in parallel (e.g., 2, 3, 4, 5, 6, 7, 8, 9, 10, or more parallel reactions). As one non-limiting example, in some embodiments, 4 parallel reactions of 12 target nucleic acids are performed.

The term “asymmetric PCR” refers to a type of PCR that preferentially amplifies one strand of an original target nucleic acid (e.g., DNA) compared to the other strand. Asymmetric PCR can be performed, e.g., using a primer pair in which the concentration of a first primer (the “excess” primer) for a chosen strand is higher than the concentration of a second primer (the “limiting” primer). Asymmetric PCR can result in generation of an excess of single-stranded DNA of a chosen strand. See, e.g., Gyllensten et al. Proc. Nat'l Acad. Sci. USA 85 (20): 7652-7652, 1988. An example of asymmetric PCR is linear-after-the-exponential (LATE)-PCR (see, e.g., Sanchez et al. Proc. Nat'l Acad. Sci USA 101 (7): 1933-1938, 2004).

The term “symmetric PCR” refers to a type of PCR that amplifies both strands of an original target nucleic acid (e.g., DNA) to similar degrees. Symmetric PCR can be performed, e.g., using a primer pair in which the concentration of a first primer is equal (or approximately equal) to the concentration of the second primer.

By “analyte” is meant a substance or a constituent of a sample to be analyzed. Exemplary analytes include one or more species of one or more of the following: a nucleic acid, an oligonucleotide, RNA (e.g., mRNA), DNA, a protein, a peptide, a polypeptide, an amino acid, an antibody, a carbohydrate, a polysaccharide, glucose, a lipid, a gas (e.g., oxygen or carbon dioxide), an electrolyte (e.g., sodium, potassium, chloride, bicarbonate, blood urea nitrogen (BUN), magnesium, phosphate, calcium, ammonia, lactate), a lipoprotein, cholesterol, a fatty acid, a glycoprotein, a proteoglycan, a lipopolysaccharide, a cell surface marker (e.g., a cell surface protein of a pathogen), a cytoplasmic marker (e.g., CD4/CD8 or CD4/viral load), a therapeutic agent, a metabolite of a therapeutic agent, a marker for the detection of a weapon (e.g., a chemical or biological weapon), an organism, a pathogen, a pathogen byproduct, a parasite (e.g., a protozoan or a helminth), a protist, a fungus (e.g., yeast or mold), a bacterium, an actinomycete, a cell (e.g., a whole cell, a tumor cell, a stem cell, a white blood cell, a T cell (e.g., displaying CD3, CD4, CD8, IL2R, CD35, or other surface markers), or another cell identified with one or more specific markers), a virus, a prion, a plant component, a plant by-product, algae, an algae by-product, plant growth hormone, an insecticide, a man-made toxin, an environmental toxin, an oil component, and components derived therefrom. In particular embodiments, the analyte is a nucleic acid (e.g., DNA or RNA (e.g., mRNA)). In particular embodiments, the analyte is a DNA.

A “biological sample” is a sample obtained from a subject including but not limited to blood (e.g., whole blood, processed whole blood (e.g., a crude whole blood lysate), serum, plasma, and other blood derivatives), bloody fluids (e.g., wound exudate, phlegm, bile, and the like), cerebrospinal fluid (CSF), urine, synovial fluid, breast milk, sweat, tears, saliva, semen, feces, vaginal fluid or tissue, sputum (e.g., purulent sputum and bloody sputum), nasopharyngeal aspirate or swab, lacrimal fluid, mucous, or epithelial swab (buccal swab), tissues (e.g., tissue biopsies (e.g., skin biopsies (e.g., from wounds, burns, or tick bites), muscle biopsies, or lymph node biopsies)), including tissue homogenates), organs, bones, teeth, among others. In several embodiments, the biological sample contains cells and/or cell debris derived from the subject from which the sample was obtained. In particular embodiments, the subject is a host of a pathogen, and the biological sample obtained from the subject includes subject (host)-derived cells and/or cell debris, as well as one or more pathogen cells.

A “biomarker” is a biological substance that can be used as an indicator of a particular disease state or particular physiological state of an organism, generally a biomarker is a protein or other native compound measured in bodily fluid whose concentration reflects the presence or severity or staging of a disease state or dysfunction, can be used to monitor therapeutic progress of treatment of a disease or disorder or dysfunction, or can be used as a surrogate measure of clinical outcome or progression. In some embodiments, the biomarker is a nucleic acid (e.g., RNA (e.g., mRNA) or DNA).

As used herein, a “fluorescent probe” is a probe containing a fluorophore. The probe may be, for example, an oligonucleotide hybridization probe, a molecular beacon, a SCORPION® probe, a hydrolysis probe, or a FRET hybridization probe.

The term “molecular beacon” as used herein refers to a detectable molecule, wherein the detectable property of the molecule is detectable only under certain specific conditions, thereby enabling it to function as a specific and informative signal. Non-limiting examples of detectable properties are optical properties, such as fluorescence, electrical properties, magnetic properties, chemical properties and time or speed through an opening of known size. A molecular beacon can include a single-stranded oligonucleotide capable of forming a stem-loop structure.

As used herein, a “hydrolysis probe” contains an oligonucleotide designed to bind a region of interest between the binding sites for PCR amplification primers, where the oligonucleotide includes a marker. For example, for a hydrolysis probe containing a fluorophore, when the DNA polymerase reaches the bound hydrolysis probe, the 5′-3′ exonuclease activity of the DNA polymerase degrades the hydrolysis probe, which separates the fluorescent reporter molecule from the rest of the probe allowing the reporter molecule to fluoresce.

As used herein, a “SCORPION® probe” is a sequence-specific, bi-labeled fluorescent probe/primer hybrid designed for quantitative PCR (qPCR). A SCORPION® probe sequence is held in a hairpin loop configuration by complementary stem sequences on the 5′ and 3′ sides of the probe. In one example, a fluorophore attached to the 5′-end and is quenched by a moiety joined to the 3′-end of the loop. The hairpin loop is linked to the 5′-end of a primer via a PCR stopper. The PCR stopper prevents read-through, which could lead to opening of the hairpin loop in the absence of the specific target sequence.

As used herein, “linked” means attached or bound by covalent bonds, non-covalent bonds, and/or linked via Van der Waals forces, hydrogen bonds, and/or other intermolecular forces.

A “pathogen” means an agent causing disease or illness to its host, such as an organism or infectious particle, capable of producing a disease in another organism, and includes but is not limited to bacteria, viruses, protozoa, prions, yeast and fungi or pathogen by-products. “Pathogen by-products” are those biological substances arising from the pathogen that can be deleterious to the host or stimulate an excessive host immune response, for example pathogen antigen/s, metabolic substances, enzymes, biological substances, or toxins. In some embodiments, the pathogen is a bacterial pathogen, e.g., a drug resistant bacterial pathogen, e.g., a bacterial pathogen that expresses one or more antibiotic resistance genes selected from the group consisting of NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M 14 and CTX-M 15), mefA, mefE, ermA, ermB, SHV, and TEM.

By “pathogen-associated analyte” is meant an analyte characteristic of the presence of a pathogen (e.g., a bacterium, fungus, or virus) in a sample. The pathogen-associated analyte can be a particular substance derived from a pathogen (e.g., a nucleic acid (e.g., DNA or RNA (e.g., mRNA)), protein, lipid, polysaccharide, or any other material produced by a pathogen) or a mixture derived from a pathogen (e.g., whole cells, or whole viruses). In certain instances, the pathogen-associated analyte is selected to be characteristic of the genus, species, or specific strain of pathogen being detected. Alternatively, the pathogen-associated analyte is selected to ascertain a property of the pathogen, such as resistance to a particular therapy. In some embodiments, a pathogen-associated analyte may be a target nucleic acid (e.g., DNA or RNA (e.g., mRNA)) that has been amplified.

A “subject” is an animal, preferably a mammal (including, for example, rodents (e.g., mice or rats), farm animals (e.g., cows, sheep, horses, and donkeys), pets (e.g., cats and dogs), or primates (e.g., non-human primates and humans)). In particular embodiments, the subject is a human. A subject may be a patient (e.g., a patient having or suspected of having a disease associated with or caused by a pathogen). In some embodiments, a subject is a host of one or more pathogens.

The term “drug resistance” refers to the ability of a pathogen to resist one or more effects of a therapeutic agent. For example, “antimicrobial resistance” refers to the ability of a microbe (e.g., a bacterial or fungal pathogen) to resist one or more effects of an antimicrobial agent, and “antibiotic resistance” refers to the ability of a bacterium to resist one or more effects of an antibiotic agent. Drug-resistant pathogens can be more difficult to treat than drug-sensitive pathogens. Resistance can occur naturally in pathogens, or can arise via spontaneous mutation or by gene transfer between different species. A pathogen may be become resistant to a therapeutic agent that previously was able to treat an infection caused by the pathogen. In some embodiments, a drug-resistant pathogen is able to survive or proliferate upon exposure to a concentration of a therapeutic agent that would kill or slow proliferation of a drug-sensitive pathogen.

The terms “drug resistance gene,” a “resistance gene,” a “drug resistance target nucleic acid,” or a “resistance target nucleic acid” are used interchangeably herein and refer to a gene that confers or facilitates drug (e.g., antibiotic) resistance, or a portion thereof.

For example, an “antibiotic resistance gene,” or an “antibiotic resistance target nucleic acid” refers to a gene that confers or facilitates antibiotic resistance, or a portion thereof. Exemplary antibiotic (e.g., carbapenem) resistance genes include, but are not limited to, NDM, KPC, IMP, VIM, OXA (e.g., OXA-23-like or OXA-48-like), DHA, CMY, FOX, mecA, mecC, MCR (e.g., mcr-1), vanA, vanB, CTX-M (e.g., CTX-M 14 and CTX-M 15), mefA, mefE, ermA, ermB, SHV, TEM, FKS, PDR1, and ERG11. Additional antibiotic resistance genes are described herein or are known in the art. In the literature, the enzymes encoded by these genes are typically spelled in capital letters, while the gene names are italicized. For example, the enzyme NDM is encoded by the bla_{NDM} gene. This convention generally holds for all of the beta lactamase genes (e.g., NDM, KPC, IMP, VIM, DHA, CMY, FOX, CTX-M, SHV, TEM, and OXA (e.g., OXA-23-like or OXA-48-like). In the present application, these terms are used interchangeably, and the capitalized shorthand terms, e.g., “NDM” may be used to refer to a nucleic acid for simplicity. Other resistance genes are typically italicized in the literature (mecA, mecC, vanA, vanB, mefA, mefE, ermA, ermB, FKS, PDR1, and ERG11), but in the present application, it is to be understood that italicized and non-italicized versions of these names are used interchangeably.

The terms “NDM” or “bla_{NDM}” refer to New Delhi metallo-beta-lactamase (e.g., NDM-1), as well as variants thereof, which may differ from NDM-1 by one or more amino acid substitutions, insertions, and/or deletions (including, e.g., NDM-2, NDM-3, NDM-4, NDM-5, NDM-6, NDM-7, NDM-8, NDM-9, NDM-10, NDM-11, NDM-12, NDM-13, NDM-14, NDM-15, NDM-16, NDM-17, NDM-18, NDM-19, NDM-20, NDM-21, NDM-22, NDM-23, NDM-24, and NDM-27).

The terms “KPC” or “bla_{KPC}” refer to K. pneumoniae carbapenemase (e.g., KPC-2), as well as variants thereof, which may differ from KPC-2 by one or more amino acid substitutions, insertions, and/or deletions (including, e.g., KPC-3, KPC-4, KPC-5, KPC-6, KPC-7, KPC-8, KPC-10, KPC-11, KPC-12, KPC-13, KPC-14, KPC-15, KPC-16, KPC-17, KPC-18, KPC-19, KPC-21, KPC-22, KPC-23, KPC-24, KPC-25, KPC-26, KPC-27, KPC-28, KPC-29, KPC-30, KPC-31, KPC-32, KPC-33, KPC-34, KPC-35, KPC-36, KPC-37, KPC-38, and KPC-39).

The terms “IMP” or “bla_{MP}” refers to a metallo-beta-lactamase active on imipenem, including IMP-1, as well as variants thereof, which may differ from IMP-1 by one or more amino acid substitutions, insertions, and/or deletions (including, e.g., IMP-2, IMP-3, IMP-4, IMP-5, IMP-6, IMP-7, IMP-8, IMP-9, IMP-10, IMP-11, IMP-12, IMP-13, IMP-14, IMP-15, IMP-16, IMP-17, IMP-18, IMP-19, IMP-20, IMP-21, IMP-22, IMP-23, IMP-24, IMP-25, IMP-26, IMP-27, IMP-28, IMP-29, IMP-30, IMP-31, IMP-32, IMP-33, IMP-34, IMP-35, IMP-37, IMP-38, IMP-40, IMP-41, IMP-42, IMP-43, IMP-44, IMP-45, IMP-48, IMP-49, IMP-51, IMP-52, IMP-53, IMP-54, IMP-55, IMP-56, IMP-58, IMP-59, IMP-60, IMP-61, IMP-62, IMP-63, IMP-64, IMP-66, IMP-67, IMP-68, IMP-70, IMP-71, IMP-73, IMP-74, IMP-75, IMP-76, IMP-77, IMP-78, IMP-79, and IMP-80).

The terms “VIM” or “bla_{VIM}” refers to Verona integron-encoded metallo-beta-lactamase, also referred to as VIM-1, as well as variants thereof, which may differ from VIM-1 by one or more amino acid substitutions, insertions, and/or deletions (including, e.g., VIM-2, VIM-3, VIM-4, VIM-5, VIM-6, VIM-7, VIM-8, VIM-9, VIM-10, VIM-11, VIM-12, VIM-13, VIM-14, VIM-15, VIM-16, VIM-17, VIM-18, VIM-19, VIM-20, VIM-23, VIM-24, VIM-25, VIM-26, VIM-27, VIM-28, VIM-29, VIM-30, VIM-31, VIM-32, VIM-33, VIM-34, VIM-35, VIM-36, VIM-37, VIM-38, VIM-39, VIM-40, VIM-41, VIM-42, VIM-43, VIM-44, VIM-45, VIM-46, VIM-47, VIM-49, VIM-50, VIM-51, VIM-52, VIM-53, VIM-54, VIM-56, VIM-57, VIM-58, VIM-59, VIM-60, VIM-61, and VIM-62).

The term “OXA” or “bla_{OXA}” refers to a group of carbapenem-hydrolyzing class D beta lactamases originally named for their activity against oxacillin. Exemplary OXA beta lactamases include, without limitation, OXA-23-like and OXA-48-like beta lactamases.

The term “OXA-23-like” refers to a group of carbapenem-hydrolyzing class D beta lactamases. This group encompasses OXA-23 (also referred to as bla_{OXA}-23) as well as OXA-23-like variants, which may differ from OXA-23 by one or more amino acid substitutions, insertions, and/or deletions (including, e.g., OXA-27, OXA-49, OXA-73, OXA-103, OXA-133, OXA-146, OXA-165, OXA-166, OXA-167, OXA-168, OXA-169, OXA-170, OXA-171, OXA-225, OXA-366, OXA-398, OXA-422, OXA-423, OXA-435, OXA-440, OXA-482, OXA-483, OXA-565, and OXA-657).

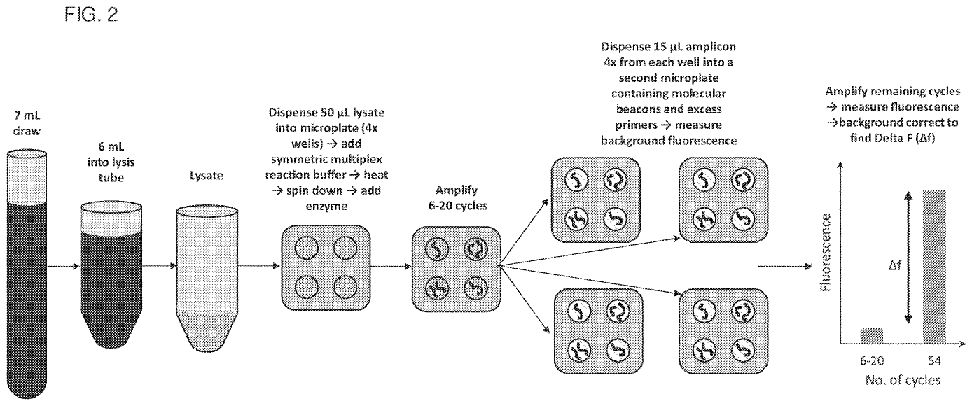

The term “OXA-48-like” refers to a group of carbapenem-hydrolyzing class D beta lactamases. This group encompasses OXA-48 (also referred to as bla_{OXA}-48) as well as OXA-48-like variants, which may differ from OXA-48 by one or more amino acid substitutions, insertions, and/or deletions (including, e.g., OXA-162, OXA-163, OXA-181, OXA-199, OXA-204, OXA-232, OXA-244, OXA-245, OXA-247, OXA-252, OXA-370, OXA-405, OXA-416, OXA-438, OXA-439, OXA-484, OXA-505, OXA-514, OXA-515, OXA-517, OXA-519, OXA-538, OXA-546, OXA-547, OXA-566, OXA-567). A sequence alignment of OXA-48 and OXA-48-like variants is shown in FIG. 2 of Poirel et al. J. Antimicrob. Chemother. 67 (7): 1597-606, 2012.

The terms “DHA” or “bla_{DHA}” refer to plasmid-mediated Dhahran beta-lactamase, including DHA-1, as well as variants thereof, which may differ from DHA-1 by one or more amino acid substitutions, insertions, and/or deletions (including, e.g., DHA-2, DHA-3, DHA-4, DHA-5, DHA-6, DHA-7, DHA-10, DHA-12, DHA-13, DHA-14, DHA-15, DHA-16, DHA-17, DHA-18, DHA-19, DHA-20, DHA-21, DHA-22, DHA-23, DHA-24, DHA-25, DHA-26, DHA-27, and DHA-28).

The terms “CMY” or “bla_{CMY}” refers to a group of plasmid-mediated class C beta-lactamases that encode for resistance to antibiotics such as cephamycins, including CMY-2, as well as variants thereof, which may differ from CMY-2 by one or more amino acid substitutions, insertions, and/or deletions (including, e.g., CMY-4, CMY-5, CMY-6, CMY-7, CMY-12, CMY-13, CMY-14, CMY-15, CMY-16, CMY-17, CMY-18, CMY-20, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-36, CMY-37, CMY-38, CMY-39, CMY-40, CMY-41, CMY-42, CMY-43, CMY-44, CMY-45, CMY-46, CMY-47, CMY-48, CMY-49, CMY-50, CMY-51, CMY-53, CMY-54, CMY-55, CMY-56, CMY-57, CMY-58, CMY-59, CMY-60, CMY-61, CMY-62, CMY-63, CMY-64, CMY-65, CMY-66, CMY-67, CMY-68, CMY-69, CMY-70, CMY-71, CMY-72, CMY-73, CMY-74, CMY-75, CMY-76, CMY-77, CMY-78, CMY-79, CMY-80, CMY-81, CMY-82, CMY-83, CMY-84, CMY-85, CMY-86, CMY-87, CMY-89, CMY-90, CMY-93, CMY-94, CMY-95, CMY-96, CMY-97, CMY-99, CMY-100, CMY-101, CMY-102, CMY-103, CMY-104, CMY-105, CMY-106, CMY-107, CMY-108, CMY-109, CMY-110, CMY-111, CMY-112, CMY-113, CMY-114, CMY-115, CMY-116, CMY-117, CMY-118, CMY-119, CMY-121, CMY-122, CMY-124, CMY-125, CMY-127, CMY-128, CMY-129, CMY-130, CMY-131, CMY-132, CMY-133, CMY-134, CMY-135, CMY-138, CMY-139, CMY-140, CMY-141, CMY-142, CMY-143, CMY-144, CMY-145, CMY-146, CMY-147, CMY-148, CMY-149, CMY-150, CMY-151, CMY-152, CMY-153, CMY-154, CMY-155, CMY-156, CMY-158, CMY-159, CMY-160, CMY-161, CMY-162, CMY-163, and BIL-1).

The term “mecA” refers to a gene that confers resistance to antibiotics such as methicillin and other beta-lactam antibiotics. Methicillin-resistant S. aureus (MRSA) is a commonly known carrier of the mecA gene. An exemplary mecA gene is provided in the NCBI AMR database under accession number NG_047937.1.

The term “mecC” refers to a gene that confers resistance to antibiotics such as methicillin and other beta-lactam antibiotics. mecC is a divergent homologue of mecA, and is also known as mecA_{LGA251}.

The term “vanA” refers to a class of antibiotic resistance genes conferring resistance to antibiotics such as vancomycin.

The term “vanB” refers to a class of antibiotic resistance genes conferring resistance to antibiotics such as vancomycin.

The terms “CTX-M” or “bla_{CTX-M}” refer to a class of extended spectrum beta-lactamases active on cefotaxime and first discovered in Munich. For example, in some embodiments the CTX-M belongs to the “CTX-M 14” group (also referred to as the CTX-M 9 group), which includes CTX-M-9, CTX-M-13, CTX-M-14, CTX-M-16, CTX-M-17, CTX-M-19, CTX-M-21, CTX-M-24, CTX-M-27, CTX-M-46, CTX-M-47, CTX-M-48, CTX-M-49, CTX-M-50, CTX-M-64, CTX-M-73, CTX-M-81, CTX-M-87, CTX-M-90, CTX-M-93, CTX-M-98, CTX-M-102, CTX-M-104, CTX-M-121, CTX-M-125, CTX-M-148, CTX-M-168, CTX-M-198, CTX-M-199, CTX-M-201, CTX-M-214, CTX-M-221, and CTX-M-223. In other embodiments, the CTX-M belongs to the “CTX-M 15” group (also referred to as the CTX-M 1 group), which includes CTX-M-1, CTX-M-3, CTX-M-10, CTX-M-12, CTX-M-15, CTX-M-22, CTX-M-23, CTX-M-28, CTX-M-29, CTX-M-30, CTX-M-32, CTX-M-33, CTX-M-36, CTX-M-42, CTX-M-53, CTX-M-54, CTX-M-55, CTX-M-61, CTX-M-66, CTX-M-69, CTX-M-71, CTX-M-72, CTX-M-80, CTX-M-82, CTX-M-101, CTX-M-114, CTX-M-116, CTX-M-117, CTX-M-144, CTX-M-166, CTX-M-170, CTX-M-178, CTX-M-179, CTX-M-180, CTX-M-181, CTX-M-182, CTX-M-186, CTX-M-187, CTX-M-188, CTX-M-189, CTX-M-190, CTX-M-197, CTX-M-206, CTX-M-207, and CTX-M-222. Other CTX-M variants are known in the art and may be detected using the approaches described herein.

The term “mefA” refers to a gene conferring resistance to antibiotics such as macrolides by encoding drug efflux pumps. The term encompasses subclasses of mefA, including mefA and mefE.

The term “erm” refers to a class of genes conferring resistance to antibiotics such as the macrolide erythromycin. The term encompasses, for example, ermA and ermB.

The terms “SHV” or “bla_{SHV}” refers to a class of beta-lactamases. The term encompasses, for example, SHV-1, as well as variants thereof, which may differ from SHV-1 by one or more amino acid substitutions, insertions, and/or deletions (including, e.g., SHV-1, SHV-1b, SHV-2, SHV-2A, SHV-3, SHV-5, SHV-7, SHV-8, SHV-9, SHV-11, SHV-12, SHV-13, SHV-14, SHV-15, SHV-16, SHV-18, SHV-24, SHV-27, SHV-28, SHV-30, SHV-31, SHV-33, SHV-34, SHV-35, SHV-36, SHV-37, SHV-38, SHV-40, SHV-41, SHV-42, SHV-43, SHV-44, SHV-45, SHV-46, SHV-48, SHV-49, SHV-50, SHV-51, SHV-52, SHV-55, SHV-56, SHV-57, SHV-59, SHV-60, SHV-61, SHV-62, SHV-63, SHV-64, SHV-65, SHV-66, SHV-67, SHV-69, SHV-70, SHV-71, SHV-72, SHV-73, SHV-74, SHV-75, SHV-76, SHV-77, SHV-78, SHV-79, SHV-80, SHV-81, SHV-82, SHV-85, SHV-86, SHV-89, SHV-92, SHV-93, SHV-94, SHV-95, SHV-96, SHV-97, SHV-98, SHV-99, SHV-100, SHV-101, SHV-102, SHV-103, SHV-104, SHV-105, SHV-106, SHV-107, SHV-108, SHV-109, SHV-110, SHV-111, SHV-115, SHV-119, SHV-120, SHV-121, SHV-128, SHV-129, SHV-132, SHV-133, SHV-134, SHV-135, SHV-137, SHV-141, SHV-142, SHV-143, SHV-144, SHV-145, SHV-146, SHV-147, SHV-148, SHV-149, SHV-150, SHV-151, SHV-152, SHV-153, SHV-154, SHV-155, SHV-156, SHV-157, SHV-158, SHV-159, SHV-160, SHV-161, SHV-162, SHV-163, SHV-164, SHV-165, SHV-168, SHV-172, SHV-173, SHV-178, SHV-179, SHV-180, SHV-182, SHV-183, SHV-185, SHV-186, SHV-187, SHV-188, SHV-189, SHV-190, SHV-191, SHV-193, SHV-194, SHV-195, SHV-196, SHV-197, SHV-198, SHV-199, SHV-200, SHV-201, SHV-202, SHV-203, SHV-204, SHV-205, SHV-206, SHV-207, SHV-208, SHV-209, SHV-210, SHV-211, SHV-212, SHV-213, SHV-214, SHV-215, SHV-216, SHV-217, SHV-218, SHV-219, SHV-220, SHV-221, SHV-222, SHV-223, SHV-224, SHV-225, SHV-226, SHV-227, and SHV-228). For a review, see Liakoipolos et al. Front. Microbiol. 7:1374, 2016, which shows an alignment of SHV-type genes.

The terms “TEM” or “bla_{TEM}” refers to a class of beta-lactamases. The term encompasses, for example, TEM-1, as well as variants thereof, which may differ from TEM-1 by one or more amino acid substitutions, insertions, and/or deletions (including, e.g., TEM-2, TEM-3, TEM-4, TEM-6, TEM-8, TEM-9, TEM-10, TEM-11, TEM-12, TEM-15, TEM-16, TEM-17, TEM-19, TEM-20, TEM-21, TEM-22, TEM-24, TEM-26, TEM-28, TEM-29, TEM-30, TEM-32, TEM-33, TEM-34, TEM-35, TEM-36, TEM-40, TEM-43, TEM-45, TEM-47, TEM-48, TEM-49, TEM-52, TEM-53, TEM-54, TEM-55, TEM-56, TEM-57, TEM-60, TEM-63, TEM-67, TEM-68, TEM-70, TEM-71, TEM-72, TEM-76, TEM-77, TEM-78, TEM-79, TEM-80, TEM-81, TEM-82, TEM-83, TEM-84, TEM-85, TEM-86, TEM-87, TEM-88, TEM-90, TEM-91, TEM-92, TEM-93, TEM-94, TEM-95, TEM-96, TEM-97, TEM-98, TEM-99, TEM-101, TEM-102, TEM-104, TEM-105, TEM-106, TEM-107, TEM-108, TEM-109, TEM-110, TEM-111, TEM-112, TEM-113, TEM-114, TEM-115, TEM-116, TEM-120, TEM-121, TEM-122, TEM-123, TEM-124, TEM-125, TEM-126, TEM-127, TEM-128, TEM-129, TEM-130, TEM-131, TEM-132, TEM-133, TEM-134, TEM-135, TEM-136, TEM-137, TEM-138, TEM-139, TEM-141, TEM-142, TEM-143, TEM-144, TEM-145, TEM-146, TEM-147, TEM-148, TEM-149, TEM-150, TEM-151, TEM-152, TEM-153, TEM-154, TEM-155, TEM-156, TEM-157, TEM-158, TEM-159, TEM-160, TEM-162, TEM-163, TEM-164, TEM-166, TEM-167, TEM-168, TEM-169, TEM-171, TEM-176, TEM-177, TEM-178, TEM-181, TEM-182, TEM-183, TEM-184, TEM-185, TEM-186, TEM-187, TEM-188, TEM-189, TEM-190, TEM-191, TEM-193, TEM-194, TEM-195, TEM-196, TEM-197, TEM-198, TEM-201, TEM-205, TEM-206, TEM-207, TEM-208, TEM-209, TEM-210, TEM-211, TEM-212, TEM-213, TEM-214, TEM-215, TEM-216, TEM-217, TEM-219, TEM-220, TEM-224, TEM-225, TEM-226, TEM-227, TEM-229, TEM-230, TEM-231, TEM-233, TEM-234, TEM-236, and TEM-237).

A “genus,” as used herein, refers to a grouping of organisms, including pathogens. In some embodiments, a genus may be a taxonomic classification, for instance, a taxonomic domain, a taxonomic kingdom, a taxonomic phylum, a taxonomic class, a taxonomic order, a taxonomic family, or a taxonomic genus. In other embodiments, a genus may be defined by any desired or suitable characteristics such as, for example, resistance to an antimicrobial agent or Gram staining (e.g., Gram negative or Gram positive). For example, the genus may be pan-Gram positive or pan-Gram negative. It is to be understood that, in some instances, a pathogen may belong to more than one genus.

A “genus-level” or “group-level” identification refers to identification of an analyte (e.g., a target nucleic acid) that provides information regarding a genus from which the analyte was obtained (e.g., a taxonomic classification, for instance, a taxonomic domain, a taxonomic kingdom, a taxonomic phylum, a taxonomic class, a taxonomic order, a taxonomic family, or a taxonomic genus). In some embodiments, a genus-level identification does not provide species-level identification.

The term “species,” as used herein, refers to a basic unit of biological classification as well as a taxonomic rank. A skilled artisan appreciates that a species may be defined based on a number of criteria, including, for example, DNA similarity, morphology, and ecological niche. The term encompasses any suitable species concept, including evolutionary species, phylogenetic species, typological species, genetic species, and reproductive species. The term also encompasses subspecies or strains.

A “species-level” identification refers to identification of an analyte (e.g., a target nucleic acid) that provides information regarding the species from which the analyte was obtained. With respect to target nucleic acids, in some embodiments, species-level identification provides information regarding nucleic acid variants (e.g., a single nucleotide polymorphism (SNP), an insertion/deletion (indel), a repetitive element, or a microsatellite repeat), which is also referred to herein as a “variant-level” identification. In some embodiments, a species-level or variant-level identification also provides a genus-level identification.

As used herein, the terms “unit” or “units,” when used in reference to thermostable nucleic acid polymerases, refer to an amount of the thermostable nucleic acid polymerase (e.g., thermostable DNA polymerase). Typically a unit is defined as the amount of enzyme that will incorporate a particular amount of dNTPs (e.g., 10-20 nmol) into acid-insoluble material in 30-60 min at 65° C.-75° C. under particular assay conditions, although each manufacturer may define units differently. Unit definitions and assay conditions for commercially-available thermostable nucleic acid polymerases are known in the art. In some embodiments, one unit of thermostable nucleic acid polymerase (e.g., Taq DNA polymerase) may be the amount of enzyme that will incorporate 15 nmol of dNTP into acid-insoluble material in 30 min at 75° C. in an assay containing 1× ThermoPol® Reaction Buffer (New England Biosciences), 200 UM dNTPs including [³H]-dTTP, and 15 nM primed M13 DNA.

It is contemplated that methods and processes of the claimed invention encompass variations and adaptations developed using information from the embodiments described herein. Throughout the description, where processes and methods are described as having, including, or including specific steps, it is contemplated that there are processes and methods according to the present invention that consist essentially of, or consist of, the recited processing steps. It should be understood that the order of steps or order for performing certain actions is immaterial, unless otherwise specified, so long as the invention remains operable. Moreover, in many instances two or more steps or actions may be conducted simultaneously.

### Analytes

Embodiments of the invention include methods and systems for detecting and/or measuring the concentration of one or more analytes in a biological sample, including but not limited to whole blood, a crude whole blood lysate, serum, or plasma. In several embodiments, the analyte may be a nucleic acid derived from an organism. In some embodiments, the nucleic acid is a target nucleic acid derived from the organism that has been amplified to form an amplicon. In some embodiments, the organism is a plant, a mammal, a microbial species, or a virus.

In several embodiments, the analyte may be derived from a microbial pathogen. In such embodiments, the biological sample may include cells and/or cell debris from the host mammalian subject as well as one or more microbial pathogen cells. In some embodiments, the analyte is derived from a Gram-negative bacterium, a Gram-positive bacterium, a fungal pathogen (e.g., a yeast (e.g., Candida spp.) or Aspergillus spp.), a protozoan pathogen, or a viral pathogen. In some embodiments, the analyte is derived from a bacterial pathogen, including Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (e.g., K. pneumoniae with resistance marker KPC), Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter koseri), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida) and Enterobacter spp. (e.g., Enterobacter cloacae). In some embodiments, the analyte is an antimicrobial resistance marker. Exemplary non-limiting antimicrobial resistance markers include vanA, vanB, mecA, mecC, IMP, CTX-M, KPC, NDM, OXA, VIM, CMY, DHA, mcr-1, and FKS. In some embodiments, the analyte is derived from a fungal pathogen, for example, Candida spp. (e.g., Candida albicans, Candida guilliermondii, Candida glabrata, Candida krusei, Candida lusitaniae, Candida parapsilosis, Candida dubliniensis, Candida auris, Candida kefyr, and Candida tropicalis) and Aspergillus spp. (e.g., Aspergillus fumigatus). In some embodiments, the analyte is derived from a protozoan pathogen such as a Babesia spp. (e.g., Babesia microti and Babesia divergens).

In some embodiments, the analyte is derived from a viral pathogen, for example, Cytomegalovirus (CMV), Epstein Barr Virus, BK Virus, Hepatitis virus, Herpes virus, Respiratory syncytial virus (RSV), Influenza virus, Human Metapneumovirus (hMPV), Rhinovirus, Parainfluenza virus, Adenovirus, or Coronavirus (e.g., SARS-COV-2).

In some embodiments, the viral pathogen is CMV. In some embodiments, the viral pathogen is Epstein Barr Virus. In some embodiments, the viral pathogen is BK virus. In some embodiments, the viral pathogen is a Hepatitis virus, for example, a Hepatitis B virus or a Hepatitis C virus. In some embodiments, the viral pathogen is a Herpes virus. In some embodiments, the viral pathogen is a Herpes simplex virus (HSV), for example, HSV1, or HSV2. In other embodiments, the viral pathogen is Human Herpes Virus 6 or Human Herpes Virus 8. In other embodiments, the viral pathogen is RSV. In some embodiments, the viral pathogen an Influenza virus, for example, Influenza A, Influenza A subtype H1, Influenza A subtype H3, or Influenza B. In other embodiments, the viral pathogen is a Parainfluenza virus, for example, Parainfluenza 1 virus, Parainfluenza 2 virus, or Parainfluenza 3 virus. In some embodiments, the viral pathogen is hMPV. In some embodiments, the viral pathogen is a Rhinovirus. In some embodiments, the viral pathogen is an Adenovirus. In some embodiments, the viral pathogen is a Coronavirus, for example, SARS-COV-2.

In some embodiments, a pathogen-associated analyte may be a nucleic acid derived from any of the organisms described above, for example, DNA or RNA (e.g., mRNA). In some embodiments, the nucleic acid is a target nucleic acid derived from the organism that has been amplified to form an amplicon. In some embodiments, the target nucleic acid may be a multi-copy locus. Use of a target nucleic acid derived from a multi-copy locus, in particular in methods involving amplification, may lead to an increase in sensitivity in the assay. Exemplary multi-copy loci may include, for example, ribosomal DNA (rDNA) operons and multi-copy plasmids. In other embodiments, the target nucleic acid may be a single-copy locus. In particular embodiments, the target nucleic acid may be derived from an essential locus, for example, an essential house-keeping gene. In particular embodiments, the target nucleic acid may be derived from a locus that is involved in virulence (e.g., a virulence gene). In any of the above embodiments, a locus may include a gene and/or an intragenic region, for example, an internally transcribed sequence (ITS) between rRNA genes (e.g., ITS1, between the 16S and 23S rRNA genes, or ITS2, between the 5S and 23S rRNA genes).

In some embodiments, a target nucleic acid may be (a) species-specific, (b) species-inclusive (in other words, present in all strains or subspecies of a given species), (c) compatible with an amplification/detection protocol, and/or (d) present in multiple copies. In particular embodiments, a target nucleic acid is chromosomally-encoded, which can help avoid loss by, for example, plasmid exchange and plasmid curing/transduction events.

### Medical Conditions

The methods of the invention can also be used to monitor and diagnose diseases and other medical conditions. In some embodiments, the methods of the invention may be used to monitor and diagnose disease in a multiplexed, automated, no sample preparation system.

The methods and systems of the invention can be used to identify and monitor the pathogenesis of disease in a subject, to select therapeutic interventions, and to monitor the effectiveness of the selected treatment. For example, for a patient having or at risk of bacteremia and/or sepsis, the methods and systems of the invention can be used to identify the infectious pathogen, pathogen load, and to monitor white blood cell count and/or biomarkers indicative of the status of the infection. The identity of the pathogen can be used to select an appropriate therapy. In some embodiments, the methods may further include administering a therapeutic agent following monitoring or diagnosing an infectious disease. The therapeutic intervention (e.g., a particular antibiotic agent) can be monitored as well to correlate the treatment regimen to the circulating concentration of antibiotic agent and pathogen load to ensure that the patient is responding to treatment.

Exemplary diseases that can be diagnosed and/or monitored by the methods and systems of the invention include diseases caused by or associated with microbial pathogens (e.g., bacterial infection or fungal infection), Lyme disease, bloodstream infection (e.g., bacteremia or fungemia), pneumonia, peritonitis, osteomyeletis, meningitis, empyema, urinary tract infection, sepsis, septic shock, and septic arthritis), diseases that may manifest with similar symptoms to diseases caused by or associated with microbial pathogens (e.g., SIRS), and diseases caused by viral pathogens.

For example, the methods and systems of the invention may be used to diagnose and/or monitor a disease caused by any suitable pathogen, including the following non-limiting examples of pathogens: bacterial pathogens, including Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (e.g., K. pneumoniae with resistance marker KPC), Klebsiella aerogenes, and Klebsiella oxytoca), Pseudomonas spp. (e.g., Pseudomonas aeruginosa), Staphylococcus spp. (e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter koseri), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), and Enterobacter spp. (e.g., Enterobacter aerogenes and Enterobacter cloacae); fungal pathogens including but not limited to Candida spp. (e.g., Candida albicans, Candida guilliermondii, Candida glabrata, Candida krusei, Candida lusitaniae, Candida parapsilosis, Candida dubliniensis, Candida auris, Candida kefyr, and Candida tropicalis) and Aspergillus spp. (e.g., Aspergillus fumigatus); and viral pathogens including but not limited to Cytomegalovirus (CMV), Epstein Barr Virus, BK Virus, Hepatitis B virus, Hepatitis C virus, Herpes simplex virus (HSV), HSV1, HSV2, Respiratory syncytial virus (RSV), Influenza, Influenza A, Influenza A subtype H1, Influenza A subtype H3, Influenza B, Human Herpes Virus 6, Human Herpes Virus 8, Human Metapneumovirus (hMPV), Rhinovirus, Parainfluenza 1, Parainfluenza 2, Parainfluenza 3, Adenovirus, and Coronavirus (e.g., SARS-COV-2).

In some embodiments, the pathogen may be a Borrelia spp., including Borrelia burgdorferi sensu lato (Borrelia burgdorferi, Borrelia afzelii, and Borrelia garinii) species, Borrelia americana, Borrelia andersonii, Borrelia bavariensis, Borrelia bissettii, Borrelia carolinensis, Borrelia californiensis, Borrelia chilensis, Borrelia genomosp. 1 and 2, Borrelia japonica, Borrelia kurtenbachii, Borrelia lusitaniae, Borrelia myomatoii, Borrelia sinica, Borrelia spielmanii, Borrelia tanukii, Borrelia turdi, Borrelia valaisiana and unclassified Borrelia spp. In other embodiments, the pathogen may be selected from the following: Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Babesia spp. (including Babesia microti and Babesia divergens), Anaplasma spp. (including Anaplasma phagocytophilum), Francisella spp., (including Francisella tularensis (including Francisella tularensis subspp. holarctica, mediasiatica, and novicida)), Streptococcus spp. (including Streptococcus pneumonia), and Neisseria spp. (including Neisseria meningitidis).

### Treatment

In some embodiments, the methods further include selecting a therapeutic agent for a subject following a diagnosis. In some embodiments, the methods further include administering a therapeutic agent to a subject following a diagnosis. Typically, the identification of a particular pathogen in a biological sample obtained from the subject (e.g., a complex sample containing host cells and/or cell debris, e.g., blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), or sputum) will guide the selection of the appropriate therapeutic agent.

For example, for a bacterial infection (e.g., bacteremia), a therapy may include an antibiotic. In some instances, an antibiotic may be administered orally. In other instances, the antibiotic may be administered intravenously. Exemplary non-limiting antibiotics that may be used in the methods of the invention include but are not limited to, acrosoxacin, amifioxacin, amikacin, amoxycillin, ampicillin, aspoxicillin, azidocillin, azithromycin, aztreonam, balofloxacin, benzylpenicillin, biapenem, brodimoprim, cefaclor, cefadroxil, cefatrizine, cefcapene, cefdinir, cefetamet, ceftmetazole, cefoxitin, cefprozil, cefroxadine, ceftarolin, ceftazidime, ceftibuten, ceftobiprole, cefuroxime, cephalexin, cephalonium, cephaloridine, cephamandole, cephazolin, cephradine, chlorquinaldol, chlortetracycline, ciclacillin, cinoxacin, ciprofloxacin, clarithromycin, clavulanic acid, clindamycin, clofazimine, cloxacillin, colistin, danofloxacin, dapsone, daptomycin, demeclocycline, dicloxacillin, difloxacin, doripenem, doxycycline, enoxacin, enrofloxacin, erythromycin, fleroxacin, flomoxef, flucloxacillin, flumequine, fosfomycin, gentamycin, isoniazid, imipenem, kanamycin, levofloxacin, linezolid, mandelic acid, mecillinam, meropenem, metronidazole, minocycline, moxalactam, mupirocin, nadifloxacin, nafcillin, nalidixic acid, netilmycin, netromycin, nifuirtoinol, nitrofurantoin, nitroxoline, norfloxacin, ofloxacin, oxacillin, oxytetracycline, panipenem, pefloxacin, phenoxymethylpenicillin, pipemidic acid, piromidic acid, pivampicillin, pivmecillinam, polymixin-b, prulifloxacin, rufloxacin, sparfloxacin, sulbactam, sulfabenzamide, sulfacytine, sulfametopyrazine, sulphacetamide, sulphadiazine, sulphadimidine, sulphamethizole, sulphamethoxazole, sulphanilamide, sulphasomidine, sulphathiazole, teicoplanin, temafioxacin, tetracycline, tetroxoprim, tigecycline, tinidazole, tobramycin, tosufloxacin, trimethoprim, vancomycin, and pharmaceutically acceptable salts or esters thereof.

In another example, for a fungal infection, a treatment may include an antifungal agent. Exemplary antifungal agents include, but are not limited to, polyenes (e.g., amphotericin B, candicidin, filipin, hamycin, natamycin, nystatin, and rimocidin), azoles (e.g., imidazoles such as bifonazole, butoconazole, clotrimazole, eberconazole, econazole, fenticonazole, flutrimazole, isoconazole, ketoconazole, luliconazole, miconazole, omoconazole, oxiconazole, sertaconazole, sulconazole, and tioconazole; triazoles such as albaconazole, efinaconazole, epoxiconazole, fluconazole, isavuconazole, itraconazole, posaconazole, propiconazole, ravuconazole, terconazole, and voriconazole; and thiazoles such as abafungin), allylamines (e.g., amorolfin, butenafine, naftifine, and terbinafine), echinocandins (e.g., anidulafungin, caspofungin, and micafungin), and other antifungal agents including but not limited to benzoic acid, ciclopirox olamine, 5-flucytosin, griseofulvin, haloprogin, tolnaftate, aminocandin, chlordantoin, chlorphenesin, nifuroxime, undecylenic acid, crystal violet, and pharmaceutically acceptable salts or esters thereof.

In a further example, for a viral infection, a treatment may include an antiviral agent.

In some embodiments, a method of treatment may include administering a treatment to an asymptomatic patient, for example, based on the detection and/or identification of a pathogen present in a biological sample derived from the patient by the methods of the invention. In other embodiments, a method of treatment may include administering a treatment to a symptomatic patient based on the detection of identification of a pathogen present in a biological sample derived from the patient by the methods of the invention. In several embodiments, the biological sample may contain cells and/or cell debris derived from both the host subject and a pathogen, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), or sputum (e.g., purulent sputum or bloody sputum). In some embodiments, the biological sample is blood (e.g., whole blood, a crude whole blood lysate, serum, or plasma) or a bloody fluid (e.g., wound exudate, phlegm, bile, and the like). In particular embodiments, the biological sample is whole blood. In other particular embodiments, the biological sample is a crude whole blood lysate.

In some embodiments, the treatment selected for a patient is based on the detection and/or identification of a pathogen by the methods of the invention. Appropriate treatments for different pathogen species are known in the art. In one example, if a Gram positive bacterium is detected in a biological derived from a patient, a method of treatment may involve administration of vancomycin. In another example, if a Gram negative bacterium is detected in a biological derived from a patient, a method of treatment may involve administration of pipercillin-tazobactam. In another example, in some embodiments, if an Acinetobacter spp. (e.g., Acinetobacter baumannii) is detected in a biological sample derived from a patient, a method of treatment may involve administration of colistin, meropenem, and/or gentamicin. In another example, in some embodiments, if a Klebsiella spp. (e.g., Klebsiella pneumoniae) is detected in a biological sample derived from a patient, a method of treatment may involve administration of meropenem. In yet another example, in some embodiments, if a Pseudomonas spp. (e.g., Pseudomonas aeruginosa) is detected in a biological sample derived from a patient, a method of treatment may involve administration of pipercillin-tazobactam. In a further example, in some embodiments, if an Escherichia spp. (e.g., Escherichia coli) is detected in a biological sample derived from a patient, a method of treatment may involve administration of meropenem. In another example, in some embodiments, if an Enterococcus spp. (e.g., Enterococcus faecium) is detected in a biological sample derived from a patient, a method of treatment may involve administration of daptomycin.

### Assay Reagents

The methods described herein may include any suitable reagents, for example, surfactants, buffer components, additives, chelating agents, and the like. Any suitable surfactant may be used. The surfactant may be selected from a wide variety of soluble non-ionic surface active agents including surfactants that are generally commercially available under the IGEPAL® trade name from GAF Company. The IGEPAL® liquid non-ionic surfactants are polyethylene glycol p-isooctylphenyl ether compounds and are available in various molecular weight designations, for example, IGEPAL® CA720, IGEPAL® CA630, and IGEPAL® CA890. Other suitable non-ionic surfactants include those available under the trade name TETRONIC® 909 from BASF Corporation. This material is a tetra-functional block copolymer surfactant terminating in primary hydroxyl groups. Suitable non-ionic surfactants are also available under the ALPHONIC® trade name from Vista Chemical Company and such materials are ethoxylates that are non-ionic biodegradables derived from linear primary alcohol blends of various molecular weights. The surfactant may also be selected from poloxamers, such as polyoxyethylene-polyoxypropylene block copolymers, such as those available under the trade names SYNPERONIC® PE series (ICI), PLURONIC® series (BASF), Supronic, MONOLAN®, PLURACARE®, and PLURODAC®, polysorbate surfactants, such as TWEEN® 20 (PEG-20 sorbitan monolaurate), and glycols such as ethylene glycol and propylene glycol. In some embodiments, an eco-friendly or biodegradable surfactant may be used (e.g., TERGITOL™ 15-S-9, TERGITOL™ 15-S-7, TERGITOL™ 15-S-30, TERGITOL™ 15-S-40, TERGITOL™ 15-S-5, TERGITOL™ 15-S-15, TERGITOL™ 15-S-3, TERGITOL™ 15-S-12, TERGITOL™ 15-S-20, TRITON™ GC-110, ECOSURFT EH-3, ECOSURFT EH-6, ECOSURFT EH-9, ECOSURF™ SA-4, ECOSURF™ SA-7, or a combination thereof). For example, in some embodiments, the surfactant may be TERGITOL™ 15-S-9, TERGITOL™ 15-S-7, or ECOSURF™ EH-9.

Such surfactants (e.g., non-ionic surfactants) may be selected to provide an appropriate amount of detergency for an assay without having a deleterious effect on assay reactions. In particular, surfactants may be included in a reaction mixture for the purpose of suppressing non-specific interactions among various ingredients of the aggregation assays of the invention. The surfactants (e.g., non-ionic surfactants) are typically added to the liquid sample prior in an amount from 0.01% (w/w) to 5% (w/w).

The surfactants (e.g., non-ionic surfactants) may be used in combination with one or more proteins (e.g., albumin, fish skin gelatin, lysozyme, or transferrin) also added to the liquid sample prior in an amount from 0.01% (w/w) to 5% (w/w).

Furthermore, the assays and methods of the invention can include additional suitable buffer components (e.g., Tris base, selected to provide a pH of about 7.8 to 8.2 in the reaction milieu); and chelating agents to scavenge cations (e.g., ethylene diamine tetraacetic acid (EDTA), EDTA disodium, citric acid, tartaric acid, glucuronic acid, saccharic acid or suitable salts thereof).

The methods may utilize one or more multi-well plates. Multi-well plates for PCR, and seals for such plates, are available from commercial sources such as Thermo Fischer Scientific and Bio-Rad. High-profile, low-profile, non-skirted, semi-skirted, skirted and bar-coded plates are available with a variety of well numbers. The multi-well plates may include any suitable number of wells. For example, 4-well, 6-well, 8-well, 10-well, 12-well, 14-well, 16-well, 18-well, 20-well, 22-well, 24-well, 48-well, 96-well, and 384-well plates may be used in the amplification methods described herein. The wells in the plates may be clear, white, or black. The wells may be thin-walled and may have a round bottom. In some embodiments, the multi-well plate is a 4-well plate.

One example of a seal for a PCR plate is the Slit Seal (BioChromato) which provides an instant, self-closing plate seal. This adhesive-free seal includes pre-cut slits made of silicone and PET and allows easy insertion and withdrawal of pipette tips and sampling needles without catching and dragging.

In another example, no plastic seal is required when the sample in each well is covered with oil (e.g., mineral oil or silicone oil).

### Fluorescent Probes

The methods described herein may include use of a fluorescent probe, e.g., a pathogen-specific fluorescent probe, for example, a molecular beacon, a SCORPION® probe, a FRET hybridization probe, or a hydrolysis probe. Any suitable fluorescent probe(s) may be used in the methods described herein.

In some embodiments, a molecular beacon can be a single-stranded oligonucleotide capable of forming a stem-loop structure, where the loop sequence may be complementary to a target nucleic acid sequence of interest, e.g., a pathogen sequence, and is flanked by short complementary arms that can form a stem. The oligonucleotide may be labeled at one end with a fluorophore and at the other end with a quencher molecule. In the stem-loop conformation, energy from the excited fluorophore is transferred to the quencher, through long-range dipole-dipole coupling similar to that seen in fluorescence resonance energy transfer, or FRET, and released as heat instead of light. When the loop sequence is hybridized to a specific target sequence, the two ends of the molecule are separated and the energy from the excited fluorophore is emitted as light, generating a detectable signal. Molecular beacons offer the added advantage that removal of excess probe is unnecessary due to the self-quenching nature of the unhybridized probe. In some embodiments, molecular beacon probes can be designed to discriminate or tolerate mismatches between the loop and target sequences by modulating the relative strengths of the loop-target hybridization and stem formation. As referred to herein, the term “mismatched nucleotide” or a “mismatch” refers to a nucleotide that is not complementary to the target sequence at that position or positions. A probe may have at least one mismatch, but can also have 2, 3, 4, 5, 6 or 7 or more mismatched nucleotides.

Non-limiting examples of fluorophores include dyes that can be synthesized or obtained commercially (e.g. Operon Biotechnologies, Huntsville, Ala.), as well as quantum dots (e.g., graphene quantum dots). A large number of dyes (more than 50) are available for application in fluorescence excitation applications. These dyes include those from the ATTO, fluorescein, rhodamine Alexa® Fluor, Biodipy, Coumarin, and Cyanine dye families. Specific examples of fluorophores include, but are not limited to, ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740, FAM or 6-FAM (6-carbolfuorescein), TET (tetrachlorofluorescein), JOE (6-carboxy-4′,5′-dichloro-2′,7′-dimethoxyfluorescein), HEX (6-carboxy-2,4,4,5,7,7-hexachlorofluorescein), TMR (tetramethylrhodamine), ROX (carboxy-X-rhodamine), X-Rhodamine, Texas Red®, LC red 640, Cy2, Cy3, Cy3B, Cy3.5, Cy5, Cy5.5, Cy7, LC red 705, Lissamine Rhodamine B, Allophycocyanin (APC), BODIPY-FL, FluorX, TruRed, PerCP, Red 613, R-Phycoerythrin (PE), NBD, Lucifer Yellow, Pacific Orange, Pacific Blue, Cascade Blue, Methoxycoumarin, Aminocoumarin, and Hydroxycoumarin. For example, in some instances, an ATTO dye (e.g., ATTO 425 or ATTO 633) is used. In some embodiments, dyes with emission maxima from 410 nm (e.g., Cascade Blue) to 775 nm (e.g., ALEXA® Fluor 750) are available and can be used.

In some embodiments, a quencher can be used for labeling oligo sequences to minimize background fluorescence or for use in fluorophore/quencher pairs, as described elsewhere herein. Quenchers are known to those of ordinary skill in the art. Non-limiting examples of quenchers include ATTO 540Q, ATTO 575Q, ATTO 580Q, ATTO 612Q, DDQ-I, Dabcyl, Eclipse, Iowa Black FQ, BHQ-1 (Black Hole Quencher® 1), QSY-7, BHQ-2 (Black Hole Quencher® 2), DDQ-II, Iowa Black RQ, QSY-21, TAMRA (tetramethyirhodamine), and BHQ-3 (Black Hole Quencher® 3), as well as gold nanoparticle quenchers and silica nanoparticle quenchers. In some embodiments, a quencher may have an absorption maximum within the range of 430 nm (e.g., DDQ-I) to 670 nm (e.g., BHQ-3).

SCORPION® probes are sequence-specific, bi-labeled fluorescent probe/primer hybrids designed for quantitative PCR (qPCR). A Scorpions Uni-Probe available from Sigma Aldrich consists of a single-stranded bi-labeled fluorescent probe sequence held in a hairpin-loop conformation with a 5′ end reporter and an internal quencher directly linked to the 5′ end of a PCR primer via a blocker. The blocker prevents the polymerase from extending the PCR primer. At the beginning of the qPCR, the polymerase extends the PCR primer and synthesizes the complementary strand of the specific target sequence. During the next cycle, the hairpin-loop unfolds and the loop-region of the probe hybridizes intramolecularly to the newly synthesized target sequence. Now that the reporter is no longer in close proximity to the quencher, fluorescence emission may take place. The fluorescent signal is detected by the qPCR instrument and is directly proportional to the amount of target DNA.

Hydrolysis probes may also be used to the methods described herein. Hydrolysis probes contain oligonucleotides designed to bind a region of interest between the binding sites for the PCR amplification primers. During the extension phase of the PCR cycle, the DNA polymerase (e.g., Taq polymerase) synthesizes the complementary strand downstream of the PCR primers. When extension reaches the bound hydrolysis probe the 5′-3′ exonuclease activity of the DNA polymerase degrades the hydrolysis probe. Cleavage of the probe separates the fluorescent reporter molecule from the rest of the probe allowing the reporter molecule to fluoresce. The DNA polymerase continues synthesizing the rest of the nascent strand. Thus, inclusion of the probe does not inhibit the PCR reaction. With subsequent PCR cycles the amount of fluorescent reporter released, and hence fluorescence, increases cumulatively.

TaqMan® probes are examples of hydrolysis probes. TaqMan® probes may include internal or terminal quenchers. In some examples, the TaqMan® probe may include a fluorophore covalently attached to the 5′-end of the oligonucleotide probe and a quencher at the 3′-end. In other examples, the TaqMan® probe may utilize an internal quencher. Several different fluorophores (e.g. 6-carbolfuorescein (FAM) or tetrachlorofluorescein (TET) and quenchers (e.g. tetramethylrhodamine (TAMRA)) are available. The quencher molecule quenches the fluorescence emitted by the fluorophore when excited by the cycler's light source via fluorescence resonance energy transfer (FRET). As long as the fluorophore and the quencher are in proximity. quenching inhibits any fluorescence signals.

TaqMan® probes are designed such that they anneal within a DNA region amplified by a specific set of primers. TaqMan® probes can be conjugated to a minor groove binder (MGB) moiety, dihydrocyclopyrroloindole tripeptide (DPI₃), in order to increase its binding affinity to the target sequence; MGB-conjugated probes have a higher melting temperature (Tm) due to increased stabilization of van der Waals forces. As the Taq polymerase extends the primer and synthesizes the nascent strand, the 5′ to 3′ exonuclease activity of the Taq polymerase degrades the probe that has annealed to the template. Degradation of the probe releases the fluorophore from it and breaks the proximity to the quencher, thus relieving the quenching effect and allowing fluorescence of the fluorophore. Hence, fluorescence detected in the quantitative PCR thermal cycler is directly proportional to the fluorophore released and the amount of DNA template present in the PCR.

FRET hybridization probes use two differently labeled oligonucleotide probes. The first oligonucleotide is labeled at the 3′-end (e.g., with fluorescein) and the second oligonucleotide is labeled at the 5′-end with a FRET acceptor (e.g., Cy5 or TAMRA). The first oligonucleotide hybridizes to the target in such a way that its 3′-end is separated from the 5′-end of the second oligonucleotide by no more than 1 base. When no complementary sequence is available, only the fluorescence of the donor is visible. If the target is present, the labeled probes will hybridize with the target and FRET can occur. The fluorescence of the acceptor is mediated by the quencher that emits fluorescence at a longer wavelength than that of the acceptor. The fluorescence intensity is proportional to the amount of POR product formed during the early exponential phase of POR (threshold value). The 3′-ends of both probes have to be protected against chain elongation during PCR.

Amplification and Detection of Nucleic Acids from Whole Blood Samples

In several embodiments, the methods and systems of the invention involve amplification of one or more nucleic acids. Amplification may be exponential or linear. A target or template nucleic acid may be either DNA or RNA (e.g., mRNA). The sequences amplified in this manner form an “amplified region” or “amplicon.” Primers and probes can be designed by those skilled in the art to target a specific template nucleic acid sequence. In certain preferred embodiments, resulting amplicons are short to allow for rapid cycling and generation of copies. The size of the amplicon can vary as needed, for example, to provide the ability to discriminate target nucleic acids from non-target nucleic acids. For example, amplicons can be less than about 1,000 nucleotides in length. Desirably the amplicons are from 50 to 500 nucleotides in length (e.g., 50 to 100, 100 to 200, 150 to 250, 300 to 400, 350 to 450, or 400 to 500 nucleotides in length). In some embodiments, more than one (e.g., 1, 2, 3, 4, 5, 6, 7, 8, 9, 10, 11, 12, 13, 14, 15, 16, 17, 18, 19, 20, or more than 20) target nucleic acids may be amplified in one reaction. In other embodiments, a single target nucleic acid may be amplified in one reaction. In some embodiments, the invention provides amplification-based nucleic acid detection assays conducted in complex samples containing cells and/or cell debris, including but not limited to blood (e.g., whole blood, a crude whole blood lysate, serum, or plasma), bloody fluids (e.g., wound exudate, phlegm, bile, and the like), tissue samples (e.g., tissue biopsies (e.g., skin biopsies, muscle biopsies, or lymph node biopsies), including homogenized tissue samples), or sputum (e.g., purulent sputum or bloody sputum). In several embodiments, the method provides methods for amplifying target nucleic acids in a biological sample that includes cells and/or cell debris derived from both a host mammalian subject and from a microbial organism, particularly a microbial pathogen.

Sample preparation typically involves removing or providing resistance for common PCR inhibitors found in complex samples containing cells and/or cell debris. Common inhibitors are listed in Table 1 (see also Wilson, Appl. Environ. Microbiol., 63:3741 (1997)). The “facilitators” in Table 1 indicate methodologies or compositions that may be used to reduce or overcome inhibition. Inhibitors typically act by either prevention of cell lysis, degradation or sequestering a target nucleic acid, and/or inhibition of a polymerase activity. The most commonly employed polymerase, Taq, is typically inhibited by the presence of 0.1% blood in a reaction. Mutant Taq polymerases have been engineered that are resistant to common inhibitors (e.g., hemoglobin and/or humic acid) found in blood (Kermekchiev et al., Nucl. Acid. Res., 37 (5): e40, (2009)). Manufacturer recommendations indicate these mutations enable direct amplification from up to 20% blood. Despite resistance afforded by the mutations, accurate real time PCR detection is complicated due to fluorescence quenching observed in the presence of blood sample (Kermekchiev et al., Nucl. Acid. Res., 37:e40 (2009)).

TABLE 1
PCR inhibitors and facilitators for overcoming inhibition.

| Sample or | | | |
|---|---|---|---|
| Specimen | | | |
| Type | Target | Inhibitor | Facilitator |
| whole blood | mammalian | >4 μl of blood/ | 1-2% blood per |
| | tissue | 100-ml reaction | reaction |
| | | mix (hemoglobin) | |
| feces | Rotavirus | unknown dilution | cellulose fiber |
| human blood | human genes | DNA binding | thermophilic protease |
| and tissue | | proteins | from Thermus strain |
| | | | rt44A |
| human | HIV type I | detergents | mineral oil |
| mononuclear | | | |
| blood cells | | | |
| bloodstain | human | unidentified heme | BSA |
| | mitochondrial | compound, hemin | |
| | DNA | | |
| blood | various | heparin | alternative |
| | | | polymerases and |
| | | | buffers, chelex, |
| | | | spermine, [Mg2+], |
| | | | glycerol, BSA, |
| | | | heparinase |

Polymerase chain reaction amplification of DNA or cDNA is a tried and trusted methodology; however, as discussed above, polymerases are inhibited by agents contained in complex biological samples containing cells and/or cell debris, including but not limited to commonly used anticoagulants and hemoglobin. Recently mutant Taq polymerases have been engineered to harbor resistance to common inhibitors found in blood and soil. Currently available polymerases, e.g., HemoKlenTaq® (New England BioLabs, Inc., Ipswich, MA) as well as OmniTaq® and OmniKlenTaq® (DNA Polymerase Technology, Inc., St. Louis, MO) are mutant (e.g., N-terminal truncation and/or point mutations) Taq polymerase that render them capable of amplifying DNA in the presence of up to 10%, 20% or 25% whole blood, depending on the product and reaction conditions (See, e.g., Kermekchiev et al. Nucl. Acids Res. 31:6139 (2003); and Kermekchiev et al., Nucl. Acid. Res., 37:e40 (2009); and see U.S. Pat. No. 7,462,475). Additionally, PHUSION® Blood Direct PCR Kits (Finnzymes Oy, Espoo, Finland), include a unique fusion DNA polymerase enzyme engineered to incorporate a double-stranded DNA binding domain, which allows amplification under conditions which are typically inhibitory to conventional polymerases such as Taq or Pfu, and allow for amplification of DNA in the presence of up to about 40% whole blood under certain reaction conditions. See Wang et al., Nucl. Acids Res. 32:1197 (2004); and see U.S. Pat. Nos. 5,352,778 and 5,500,363. Furthermore, Kapa Blood PCR Mixes (Kapa Biosystems, Woburn, MA), provide a genetically engineered DNA polymerase enzyme which allows for direct amplification of whole blood at up to about 20% of the reaction volume under certain reaction conditions. Despite these breakthroughs, direct optical detection of generated amplicons is typically not possible with existing methods since fluorescence, absorbance, and other light-based methods yield signals that are quenched by the presence of blood. See Kermekchiev et al., Nucl. Acid. Res., 37:e40 (2009). Table 2 shows a list of mutant thermostable DNA polymerases that are compatible with many types of interfering substances and that may be used in the methods of the invention for amplification and detection of target nucleic acids in biological samples containing cells and/or cell debris.

TABLE 2
Exemplary mutant thermostable DNA polymerases

| Polymerase | Reference |
|---|---|
| Klentaq ®1 | Barnes, Proc Natl Acad Sci USA. 91(6): 2216-2220, 1994. |
| Klentaq ® LA | ″ |
| Cesium Klentaq ® AC | Kermekchiev et al., Nuc. Acids Res. 31(21): 6139-6147, 2003. |
| Cesium Klentaq ® AC LA | ″ |
| Cesium Klentaq ® C | ″ |
| Cesium Klentaq ® C LA | ″ |
| Omni Klentaq ® | Kermekchiev et al. Nuc. Acids Res. 37(5): e40, 2009. |
| Omni Klentaq ® 2 | ″ |
| Omni Klentaq ® LA | ″ |
| Omni Taq | ″ |
| Omni Taq LA | ″ |
| Omni Taq 2 | ″ |
| Omni Taq 3 | ″ |
| Hemo KlenTaq ® | ″ |
| KAPA Blood DNA | KAPA Biosystems |
| Polymerase | |
| KAPA3G Plant DNA | KAPA Biosystems |
| Polymerase | |
| KAPA2G Robust DNA | KAPA Biosystems |
| Polymerase | |
| MyTaq ™ Blood-PCR Kit | Bioline |
| Phusion ® Blood DNA Kit | Thermo Scientific |
| with Hot Start Phusion II | Manage et al., Microfluid. Nanofluid. 10, 697-702, 2011. |

A variety of impurities and components of whole blood can be inhibitory to the polymerase and primer annealing. These inhibitors can sometimes lead to generation of false positives and low sensitivities. To reduce the generation of false positives and low sensitivities when amplifying and detecting nucleic acids in complex samples, it is desirable to utilize a thermal stable polymerase not inhibited by whole blood samples, for example as described above, and include one or more internal PCR assay controls (see Rosenstraus et al. J. Clin Microbiol. 36:191 (1998) and Hoofar et al., J. Clin. Microbiol. 42:1863 (2004)).

For example, the assay can include an internal control (IC) nucleic acid that contains primer binding regions identical to those of the target sequence to assure that clinical specimens are successfully amplified and detected. In some embodiments, the target nucleic acid and internal control can be selected such that each has a unique probe binding region that differentiates the internal control from the target nucleic acid. The internal control is, optionally, employed in combination with a processing positive control, a processing negative control, and a reagent control for the safe and accurate determination and identification of an infecting organism in, e.g., a whole blood clinical sample. The internal control can be an inhibition control that is designed to co-amplify with the nucleic acid target being detected. Failure of the internal inhibition control to be amplified is evidence of a reagent failure or process error. Universal primers can be designed such that the target sequence and the internal control sequence are amplified in the same reaction tube. Thus, using this format, if the target DNA is amplified but the internal control is not it is then assumed that the target DNA is present in a proportionally greater amount than the internal control and the positive result is valid as the internal control amplification is unnecessary. If, on the other hand, neither the internal control nor the target is amplified it is then assumed that inhibition of the PCR reaction has occurred and the test for that particular sample is not valid.

The assays of the invention can include one or more positive processing controls in which one or more target nucleic acids is included in the assay (e.g., each included with one or more cartridges) at 3× to 5× the limit of detection. The assays of the invention can include one or more negative processing controls consisting of a solution free of target nucleic acid (e.g., buffer alone). The purpose of the negative control is to detect carry-over contamination and/or reagent contamination. The assays of the invention can include one or more reagent controls. The reagent control will detect reagent failures in the PCR stage of the reaction (i.e. incomplete transfer of master mix to the PCR tubes).

In some embodiments, complex biological samples, which may be a liquid sample (including, for example, a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), or sputum) can be directly amplified using about 5%, about 10%, about 20%, about 25%, about 30%, about 25%, about 40%, about 45%, about 50%, about 55%, about 60%, about 65%, about 70%, or more complex liquid sample in amplification reactions, and that the resulting amplicons can be directly detected from amplification reaction using, for example, by measuring fluorescence.

While the exemplary methods described hereinafter relate to amplification using polymerase chain reaction (“PCR”), numerous other methods are known in the art for amplification of nucleic acids (e.g., isothermal methods, rolling circle methods, etc.). Those skilled in the art will understand that these other methods may be used either in place of, or together with, PCR methods. See, e.g., Saiki, “Amplification of Genomic DNA” in PCR Protocols, Innis et al., Eds., Academic Press, San Diego, Calif., pp 13-20 (1990); Wharam et al., Nucleic Acids Res. 29:E54 (2001); Hafner et al., Biotechniques, 30:852 (2001). Further amplification methods suitable for use with the present methods include, for example, reverse transcription PCR (RT-PCR), ligase chain reaction (LCR), transcription based amplification system (TAS), transcription mediated amplification (TMA), nucleic acid sequence based amplification (NASBA) method, the strand displacement amplification (SDA) method, the loop mediated isothermal amplification (LAMP) method, the isothermal and chimeric primer-initiated amplification of nucleic acid (ICAN) method, and the smart amplification system (SMAP) method. These methods, as well as others are well known in the art and can be adapted for use in conjunction with provided methods of detection of amplified nucleic acid.

The PCR method is a technique for making many copies of a specific template DNA sequence. The PCR process is disclosed in U.S. Pat. Nos. 4,683,195; 4,683,202; and 4,965,188, each of which is incorporated herein by reference. One set of primers complementary to a template DNA are designed, and a region flanked by the primers is amplified by DNA polymerase in a reaction including multiple amplification cycles. Each amplification cycle includes an initial denaturation, and up to 50 cycles of annealing, strand elongation (or extension) and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan, et al, Journal of Clinical Microbiology, 33: 556 (1995). Various modified PCR methods are available and well known in the art. Various modifications such as the “RT-PCR” method, in which DNA is synthesized from RNA using a reverse transcriptase before performing PCR; and the “TaqMan® PCR” method, in which only a specific allele is amplified and detected using a fluorescently labeled TaqMan® probe, and Taq DNA polymerase, are known to those skilled in the art. RT-PCR and variations thereof have been described, for example, in U.S. Pat. Nos. 5,804,383; 5,407,800; 5,322,770; and 5,310,652, and references described therein, which are hereby incorporated by reference; and TaqMan® PCR and related reagents for use in the method have been described, for example, in U.S. Pat. Nos. 5,210,015; 5,876,930; 5,538,848; 6,030,787; and 6,258,569, which are hereby incorporated by reference.

In some embodiments, asymmetric PCR is performed to preferentially amplify one strand of a double-stranded DNA template. Asymmetric PCR typically involves addition of an excess of the primer for the strand targeted for amplification. An exemplary asymmetric PCR condition is 300 nM of the excess primer and 75 nM of the limiting primer to favor single strand amplification. In other embodiments, 400 nM of the excess primer and 100 nM of the limiting primer may be used to favor single strand amplification.

In some embodiments, the PCR reaction is a multiplexed PCR reaction. In some embodiments, the method may include several parallel multiplexed PCR reactions (e.g., 2, 3, 4, 5, 6, 7, 8, 9, 10, or more parallel multiplexed PCR reactions). In some embodiments, the method may include 4 parallel multiplexed PCR reactions, e.g., 4 parallel reactions in with 8-16 target nucleic acids each are configured to be amplified).

In some embodiments, including embodiments that employ multiplexed PCR reactions, hot start PCR conditions may be used to reduce mis-priming, primer-dimer formation, improve yield, and/or and ensure high PCR specificity and sensitivity. A variety of approaches may be employed to achieve hot start PCR conditions, including hot start DNA polymerases (e.g., hot start DNA polymerases with aptamer-based inhibitors or with mutations that limit activity at lower temperatures) as well as hot start dNTPs (e.g., CLEANAMP™ dNTPs, TriLink Biotechnologies).

In some embodiments, a PCR reaction may include from about 6 cycles to about 55 cycles or more (e.g., about 6, 8, 10, 12, 20, 25, 30, 35, 40, 42, 44, 45, 46, 48, 50, or 55 cycles).

In some embodiments, the PCR method includes a two-stage PCR comprising a first PCR (e.g., of from 6 to 20 cycles) and a second PCR (e.g., of from 30 to 50 cycles). In some embodiments, both the first and the second PCR are asymmetric PCR. In other embodiments, the first PCR is a symmetric PCR, and the second PCR is an asymmetric PCR.

LCR is a method of DNA amplification similar to PCR, except that it uses four primers instead of two and uses the enzyme ligase to ligate or join two segments of DNA. Amplification can be performed in a thermal cycler (e.g., LCx of Abbott Labs, North Chicago, IL). LCR can be performed for example, as according to Moore et al., Journal of Clinical Microbiology 36:1028 (1998). LCR methods and variations have been described, for example, in European Patent Application Publication No. EP0320308, and U.S. Pat. No. 5,427,930, each of which is incorporated herein by reference.

The TAS method is a method for specifically amplifying a target RNA in which a transcript is obtained from a template RNA by a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS 86:1173 (1989). The TAS method has been described, for example, in International Patent Application Publication No. WO1988/010315, which is incorporated herein by reference.

Transcription mediated amplification (TMA) is a transcription-based isothermal amplification reaction that uses RNA transcription by RNA polymerase and DNA transcription by reverse transcriptase to produce an RNA amplicon from target nucleic acid. TMA methods are advantageous in that they can produce 100 to 1000 copies of amplicon per amplification cycle, as opposed to PCR or LCR methods that produce only 2 copies per cycle. TMA has been described, for example, in U.S. Pat. No. 5,399,491, which is incorporated herein by reference. NASBA is a transcription-based method which for specifically amplifying a target RNA from either an RNA or DNA template. NASBA is a method used for the continuous amplification of nucleic acids in a single mixture at one temperature. A transcript is obtained from a template RNA by a DNA-dependent RNA polymerase using a forward primer having a sequence identical to a target RNA and a reverse primer having a sequence complementary to the target RNA a on the 3′ side and a promoter sequence that recognizes T7 RNA polymerase on the 5′ side. A transcript is further synthesized using the obtained transcript as template. This method can be performed as according to Heim, et al., Nucleic Acids Res., 26:2250 (1998). The NASBA method has been described in U.S. Pat. No. 5,130,238, which is incorporated herein by reference.

The SDA method is an isothermal nucleic acid amplification method in which target DNA is amplified using a DNA strand substituted with a strand synthesized by a strand substitution type DNA polymerase lacking 5′->3′ exonuclease activity by a single stranded nick generated by a restriction enzyme as a template of the next replication. A primer containing a restriction site is annealed to template, and then amplification primers are annealed to 5′ adjacent sequences (forming a nick). Amplification is initiated at a fixed temperature. Newly synthesized DNA strands are nicked by a restriction enzyme and the polymerase amplification begins again, displacing the newly synthesized strands. SDA can be performed according to Walker, et al., PNAS, 89:392 (1992). SDA methods have been described in U.S. Pat. Nos. 5,455,166 and 5,457,027, each of which are incorporated by reference.

The LAMP method is an isothermal amplification method in which a loop is always formed at the 3′ end of a synthesized DNA, primers are annealed within the loop, and specific amplification of the target DNA is performed isothermally. LAMP can be performed according to Nagamine et al., Clinical Chemistry. 47:1742 (2001). LAMP methods have been described in U.S. Pat. Nos. 6,410,278; 6,974,670; and 7,175,985, each of which are incorporated by reference.

The ICAN method is anisothermal amplification method in which specific amplification of a target DNA is performed isothermally by a strand substitution reaction, a template exchange reaction, and a nick introduction reaction, using a chimeric primer including RNA-DNA and DNA polymerase having a strand substitution activity and RNase H. ICAN can be performed according to Mukai et al., J. Biochem. 142: 273 (2007). The ICAN method has been described in U.S. Pat. No. 6,951,722, which is incorporated herein by reference.

The SMAP (MITANI) method is a method in which a target nucleic acid is continuously synthesized under isothermal conditions using a primer set including two kinds of primers and DNA or RNA as a template. The first primer included in the primer set includes, in the 3′ end region thereof, a sequence (Ac′) hybridizable with a sequence (A) in the 3′ end region of a target nucleic acid sequence as well as, on the 5′ side of the above-mentioned sequence (Ac′), a sequence (B′) hybridizable with a sequence (Bc) complementary to a sequence (B) existing on the 5′ side of the above-mentioned sequence (A) in the above-mentioned target nucleic acid sequence. The second primer includes, in the 3′ end region thereof, a sequence (Cc′) hybridizable with a sequence (C) in the 3′ end region of a sequence complementary to the above-mentioned target nucleic acid sequence as well as a loopback sequence (D-Dc′) including two nucleic acid sequences hybridizable with each other on an identical strand on the 5′ side of the above-mentioned sequence (Cc′). SMAP can be performed according to Mitani et al., Nat. Methods, 4 (3): 257 (2007). SMAP methods have been described in U.S. Patent Application Publication Nos. 2006/0160084, 2007/0190531 and 2009/0042197, each of which is incorporated herein by reference.

The amplification reaction can be designed to produce a specific type of amplified product, such as nucleic acids that are double stranded; single stranded; double stranded with 3′ or 5′ overhangs; or double stranded with chemical ligands on the 5′ and 3′ ends. The amplified PCR product can be detected by: (i) hybridization mediated detection where the DNA of the amplified product must first be denatured; (ii) hybridization mediated detection where the particles hybridize to 5′ and 3′ overhangs of the amplified product; (iii) binding of the particles to the chemical or biochemical ligands on the termini of the amplified product, such as streptavidin functionalized particles binding to biotin functionalized amplified product.

The systems and methods of the invention can be used to perform real time PCR and provide quantitative information about the amount of target nucleic acid present in a sample (see, e.g., FIG. 52 and Example 18 of WO 2012/054639). Methods for conducting quantitative real time PCR are provided in the literature (see for example: RT-PCR Protocols. Methods in Molecular Biology, Vol. 193. Joe O'Connell, ed. Totowa, NJ: Humana Press, 2002, 378 pp. ISBN 0-89603-875-0.). Example 18 of WO 2012/054639 describes use of the methods of the invention for real time PCR analysis of a whole blood sample.

The systems and methods of the invention can be used to perform real time PCR directly in opaque samples, such as biological samples containing cells or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), or sputum, using magnetic nanoparticles modified with capture probes and magnetic separation. Using real-time PCR allows for the quantification of a target nucleic acid without opening the reaction tube after the PCR reaction has commenced.

In certain embodiments, the invention features the use of enzymes compatible with whole blood, e.g., mutant thermostable DNA polymerases including but not limited to NEB HemoKlenTaq™, DNAP OmniKlenTaq™, Kapa Biosystems whole blood enzyme, Thermo-Fisher Finnzymes PHUSION® enzyme, or any of the mutant thermostable DNA polymerases shown in Table 2.

### Sample Preparation and Cell Lysis

The methods and systems of the invention may involve sample preparation and/or cell lysis. For example, a pathogen present in a biological sample containing cells or cell debris including but not limited to blood (e.g., whole blood, a crude whole blood lysate, serum, or plasma) may be lysed prior to amplification of a target nucleic acid. Suitable lysis methods for lysing pathogen cells in a biological sample include, for example, mechanical lysis (e.g., beadbeating and sonication), heat lysis, and alkaline lysis. In some embodiments, beadbeating may be performed by adding glass beads (e.g., 0.5 mm glass beads, 0.6 mm glass beads, 0.7 mm glass beads, 0.8 mm glass beads, or 0.9 mm glass beads) to a biological sample to form a mixture and agitating the mixture. As an example, the sample preparation and cell lysis (e.g., beadbeating) may be performed using any of the approaches and methods described in WO 2012/054639. Following lysis, the sample may include cell debris derived from mammalian host cells and/or from the pathogen cell(s) present in the sample.

In some embodiments, the methods of the invention may include preparing a tissue homogenate. Any suitable method or approach known in the art and/or described herein may be used, including but not limited to grinding (e.g., mortar and pestle grinding, cryogenic mortar and pestle grinding, or glass homogenizer), shearing (e.g., blender, rotor-stator, dounce homogenizer, or French press), beating (e.g., bead beating), or sonication. In some embodiments, several approaches may be combined to prepare a tissue homogenate.

In some embodiments, the methods of the invention involve detection of one or more pathogen-associated analytes in a whole blood sample. In some embodiments, the methods may involve disruption of red blood cells (erythrocytes). In some embodiments, the disruption of the red blood cells can be carried out using an erythrocyte lysis agent (i.e., a lysis buffer, an isotonic lysis agent, or a nonionic detergent). Erythrocyte lysis buffers which can be used in the methods of the invention include, without limitation, isotonic solutions of ammonium chloride (optionally including carbonate buffer and/or EDTA), and hypotonic solutions. The basic mechanism of hemolysis using isotonic ammonium chloride is by diffusion of ammonia across red blood cell membranes. This influx of ammonium increases the intracellular concentration of hydroxyl ions, which in turn reacts with CO₂ to form hydrogen carbonate. Erythrocytes exchange excess hydrogen carbonate with chloride which is present in blood plasma via anion channels and subsequently increase in intracellular ammonium chloride concentrations. The resulting swelling of the cells eventually causes loss of membrane integrity.

Alternatively, the erythrocyte lysis agent can be an aqueous solution of nonionic detergents (e.g., nonyl phenoxypolyethoxylethanol (NP-40), 4-octylphenol polyethoxylate (TRITON™ X-100), BRIJ® 58, or related nonionic surfactants, and mixtures thereof). The erythrocyte lysis agent disrupts at least some of the red blood cells, allowing a large fraction of certain components of whole blood (e.g., certain whole blood proteins) to be separated (e.g., as supernatant following centrifugation) from the white blood cells or other cells (e.g., pathogen cells (e.g., bacterial cells and/or fungal cells)) present in the whole blood sample. Following erythrocyte lysis and centrifugation, the resulting pellet may be lysed, for example, as described above.

In some embodiments, the methods of the invention may include (a) providing a whole blood sample from a subject; (b) mixing the whole blood sample with an erythrocyte lysis agent solution to produce disrupted red blood cells; (c) following step (b), centrifuging the sample to form a supernatant and a pellet, discarding some or all of the supernatant, and resuspending the pellet to form an extract, (d) lysing cells of the extract (which may include white blood cells and/or pathogen cells) to form a lysate. In some embodiments, the method further comprises amplifying one or more target nucleic acids in the lysate. In some embodiments, the sample of whole blood is from about 0.5 to about 10 ml of whole blood, for example, 0.5 mL, 1 mL, 2 mL, 3 mL, 4 mL, 5 mL, 6 mL, 7 mL, 8 mL, 9 mL, or 10 mL of whole blood. In some embodiments, the method may include washing the pellet (e.g., with a buffer such as TE buffer) prior to resuspending the pellet and optionally repeating step (c). In some embodiments, the method may include 1, 2, 3, 4, 5, or more wash steps. In some embodiments, the method includes no more than 1 wash step. In other embodiments, the method is performed without performing any wash step. In some embodiments, the amplifying is in the presence of whole blood proteins, non-target nucleic acids, or both. In some embodiments, the amplifying may be in the presence of from 0.5 μg to 60 μg (e.g., 0.5 μg, 1 μg, 5 μg, 10 μg, 15 μg, 20 μg, 25 μg, 30 μg, 35 μg, 40 μg, 45 μg, 50 ug, 55 μg, or 60 μg) of subject (i.e., host) DNA. In some embodiments, the subject (i.e., host) DNA is from white blood cells of the subject.

Amplification and Detection of Target Nucleic Acids in Samples Containing Cells and/or Cell Debris

The invention provides methods for amplification and detection of target nucleic acids in biological samples containing cells and/or cell debris including but not limited to blood (e.g., whole blood, a crude whole blood lysate, serum, or plasma). In several embodiments, the sample contains cells and/or cell debris derived from a mammalian host subject and one or more pathogen cells.

For example, in one aspect, the invention provides 2-stage PCR methods for detection of target nucleic acids in samples containing cells and/or cell debris.

In one aspect, the invention features a method for amplifying and detecting a target pathogen nucleic acid in a biological sample suspected of containing one or more pathogen cells, the method comprising: (a) lysing one or more pathogen cells within the biological sample by contacting the biological sample with a lysis agent to form a solution containing both subject cell nucleic acid and pathogen nucleic acid; (b) amplifying the pathogen nucleic acid in the solution of step (a) by 6 to 20 cycles of PCR to form an amplified solution; (c) adding a pathogen-specific fluorescent probe to the amplified solution; (d) measuring a signal from the pathogen-specific fluorescent probe in the amplified solution of step (c) to obtain a baseline measurement; (e) further amplifying the pathogen nucleic acid in the amplified solution of step (c) by 30 to 50 cycles of PCR; (f) measuring the signal from the pathogen-specific fluorescent probe in the amplified solution of step (e); and (g) obtaining a final signal measurement by subtracting the baseline measurement from step (d) from the signal from step (f), wherein the presence of the target pathogen in the biological sample is detected based on the final signal measurement.

In some aspects, the amplifying of step (b) is symmetric PCR. In other aspects, the amplifying of step (b) is asymmetric PCR.

In some aspects, the amplifying of step (e) is symmetric PCR. In other aspects, the amplifying of step (e) is asymmetric PCR.

In some aspects, the amplifying of step (b) is symmetric PCR, and the amplifying of step (e) is asymmetric PCR.

In some aspects, the amplifying of step (b) is asymmetric PCR, and the amplifying of step (e) is asymmetric PCR.

In some aspects, the amplifying of step (b) is symmetric PCR, and the amplifying of step (e) is symmetric PCR.

In some aspects, the amplifying of step (b) is asymmetric PCR, and the amplifying of step (e) is symmetric PCR.

For example, in one aspect, the invention features a method for amplifying and detecting a target pathogen nucleic acid in a biological sample suspected of containing one or more pathogen cells, the method comprising: (a) lysing one or more pathogen cells within the biological sample by contacting the biological sample with a lysis agent to form a solution containing both subject cell nucleic acid and pathogen nucleic acid; (b) amplifying the pathogen nucleic acid in the solution of step (a) by 6 to 20 cycles of PCR to form an amplified solution, wherein the PCR is symmetric PCR; (c) adding a pathogen-specific fluorescent probe to the amplified solution; (d) measuring a signal from the pathogen-specific fluorescent probe in the amplified solution of step (c) to obtain a baseline measurement; (e) further amplifying the pathogen nucleic acid in the amplified solution of step (c) by 30 to 50 cycles of PCR, wherein the PCR is asymmetric PCR; (f) measuring the signal from the pathogen-specific fluorescent probe in the amplified solution of step (e); and (g) obtaining a final signal measurement by subtracting the baseline measurement from step (d) from the signal from step (f), wherein the presence of the target pathogen in the biological sample is detected based on the final signal measurement.

In one aspect, the invention features a method for amplifying and detecting a target pathogen nucleic acid in a biological sample suspected of containing one or more pathogen cells, the method comprising: (a) lysing one or more pathogen cells within the biological sample by contacting the biological sample with a lysis agent to form a solution containing both subject cell nucleic acid and pathogen nucleic acid, and optionally: (i) centrifuging the product of step (a) to form a supernatant and a pellet; (ii) discarding some or all of the supernatant of step (i) and optionally washing the pellet once; (iii) centrifuging the product of step (ii) to form a supernatant and a pellet; (iv) discarding some or all of the supernatant of step (iii) and mixing the pellet of step (iii) with a buffer; (b) amplifying the pathogen nucleic acid in the solution of step (a) by 6 to 20 cycles of PCR to form an amplified solution, wherein the PCR is symmetric PCR; (c) adding a pathogen-specific fluorescent probe to the amplified solution; (d) measuring a signal from the pathogen-specific fluorescent probe in the amplified solution of step (c) to obtain a baseline measurement; (e) further amplifying the pathogen nucleic acid in the amplified solution of step (c) by 30 to 50 cycles of PCR, wherein the PCR is asymmetric PCR; (f) measuring the signal from the pathogen-specific fluorescent probe in the amplified solution of step (e); and (g) obtaining a final signal measurement by subtracting the baseline measurement from step (d) from the signal from step (f), wherein the presence of the target pathogen in the biological sample is detected based on the final signal measurement.

In some embodiments, the primer concentration for the symmetric PCR of step (b) is between 50 nM to 300 nM (e.g., 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, or 300 nM). In some examples, the primer concentration for the symmetric PCR of step (b) is between 50 nM to 300 nM, between 50 nM to 250 nM, between 50 nM to 200 nM, between 50 nM to 150 nM, between 50 nM to 100 nM, between 100 nM to 300 nM, between 100 nM to 250 nM, between 100 nM to 200 nM, between 100 nM to 150 nM, between 150 nM to 300 nM, between 150 nM to 250 nM, between 150 nM to 200 nM, between 200 nM to 300 nM, between 200 nM to 250 nM, or between 250 nM to 300 nM.

In some embodiments, the excess primer concentration for the asymmetric PCR of step (e) is between 100 nM to 1000 nM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1000 nM).

In some embodiments, the excess primer concentration for the asymmetric PCR of step (e) is between 100 nM to 1000 nM, between 100 nM to 950 nM, between 100 nM to 900 nM, between 100 nM to 850 nM, between 100 nM to 800 nM, between 100 nM to 750 nM, between 100 nM to 700 nM, between 100 nM to 650 nM, between 100 nM to 600 nM, between 100 nM to 550 nM, between 100 nM to 500 nM, between 100 nM to 450 nM, between 100 nM to 400 nM, between 100 nM to 350 nM, between 100 nM to 300 nM, between 100 nM to 250 nM, between 100 nM to 200 nM, between 100 nM to 150 nM, between 150 nM to 1000 nM, between 150 nM to 950 nM, between 150 nM to 900 nM, between 150 nM to 850 nM, between 150 nM to 800 nM, between 150 nM to 750 nM, between 150 nM to 700 nM, between 150 nM to 650 nM, between 150 nM to 600 nM, between 150 nM to 550 nM, between 150 nM to 500 nM, between 150 nM to 450 nM, between 150 nM to 400 nM, between 150 nM to 350 nM, between 150 nM to 300 nM, between 150 nM to 250 nM, between 150 nM to 200 nM, between 200 nM to 1000 nM, between 200 nM to 950 nM, between 200 nM to 900 nM, between 200 nM to 850 nM, between 200 nM to 800 nM, between 200 nM to 750 nM, between 200 nM to 700 nM, between 200 nM to 650 nM, between 200 nM to 600 nM, between 200 nM to 550 nM, between 200 nM to 500 nM, between 200 nM to 450 nM, between 200 nM to 400 nM, between 200 nM to 350 nM, between 200 nM to 300 nM, between 200 nM to 250 nM, between 250 nM to 1000 nM, between 250 nM to 950 nM, between 250 nM to 900 nM, between 250 nM to 850 nM, between 250 nM to 800 nM, between 250 nM to 750 nM, between 250 nM to 700 nM, between 250 nM to 650 nM, between 250 nM to 600 nM, between 250 nM to 550 nM, between 250 nM to 500 nM, between 250 nM to 450 nM, between 250 nM to 400 nM, between 250 nM to 350 nM, between 250 nM to 300 nM, between 300 nM to 1000 nM, between 300 nM to 950 nM, between 300 nM to 900 nM, between 300 nM to 850 nM, between 300 nM to 800 nM, between 300 nM to 750 nM, between 300 nM to 700 nM, between 300 nM to 650 nM, between 300 nM to 600 nM, between 300 nM to 550 nM, between 300 nM to 500 nM, between 300 nM to 450 nM, between 300 nM to 400 nM, between 300 nM to 350 nM, between 350 nM to 1000 nM, between 350 nM to 950 nM, between 350 nM to 900 nM, between 350 nM to 850 nM, between 350 nM to 800 nM, between 350 nM to 750 nM, between 350 nM to 700 nM, between 350 nM to 650 nM, between 350 nM to 600 nM, between 350 nM to 550 nM, between 350 nM to 500 nM, between 350 nM to 450 nM, between 350 nM to 400 nM, between 400 nM to 1000 nM, between 400 nM to 950 nM, between 400 nM to 900 nM, between 400 nM to 850 nM, between 400 nM to 800 nM, between 400 nM to 750 nM, between 400 nM to 700 nM, between 400 nM to 650 nM, between 400 nM to 600 nM, between 400 nM to 550 nM, between 400 nM to 500 nM, between 400 nM to 450 nM, between 450 nM to 1000 nM, between 450 nM to 950 nM, between 450 nM to 900 nM, between 450 nM to 850 nM, between 450 nM to 800 nM, between 450 nM to 750 nM, between 450 nM to 700 nM, between 450 nM to 650 nM, between 450 nM to 600 nM, between 450 nM to 550 nM, between 450 nM to 500 nM, between 500 nM to 1000 nM, between 500 nM to 950 nM, between 500 nM to 900 nM, between 500 nM to 850 nM, between 500 nM to 800 nM, between 500 nM to 750 nM, between 500 nM to 700 nM, between 500 nM to 650 nM, between 500 nM to 600 nM, between 500 nM to 550 nM, between 550 nM to 1000 nM, between 550 nM to 950 nM, between 550 nM to 900 nM, between 550 nM to 850 nM, between 550 nM to 800 nM, between 550 nM to 750 nM, between 550 nM to 700 nM, between 550 nM to 650 nM, between 550 nM to 600 nM, between 600 nM to 1000 nM, between 600 nM to 950 nM, between 600 nM to 900 nM, between 600 nM to 850 nM, between 600 nM to 800 nM, between 600 nM to 750 nM, between 600 nM to 700 nM, between 600 nM to 650 nM, between 650 nM to 1000 nM, between 650 nM to 950 nM, between 650 nM to 900 nM, between 650 nM to 850 nM, between 650 nM to 800 nM, between 650 nM to 750 nM, between 650 nM to 700 nM, between 700 nM to 1000 nM, between 700 nM to 950 nM, between 700 nM to 900 nM, between 700 nM to 850 nM, between 700 nM to 800 nM, between 700 nM to 750 nM, between 750 nM to 1000 nM, between 750 nM to 950 nM, between 750 nM to 900 nM, between 750 nM to 850 nM, between 750 nM to 800 nM, between 800 nM to 1000 nM, between 800 nM to 950 nM, between 800 nM to 900 nM, between 800 nM to 850 nM, between 850 nM to 1000 nM, between 850 nM to 950 nM, between 850 nM to 900 nM, between 900 nM to 1000 nM, between 900 nM to 950 nM, or between 950 nM to 1000 nM.

In some embodiments, in step (b), the pathogen nucleic acid in the solution of step (a) is amplified by 6, 7, 8, 9, 10, 11, 12, 13, 14, 15, 16, 17, 18, 19, or 20 cycles of PCR (e.g., 6 to 20 cycles of PCR, 6 to 18 cycles of PCR, 6 to 16 cycles of PCR, 6 to 14 cycles of PCR, 6 to 12 cycles of PCR, 6 to 10 cycles of PCR, 6 to 8 cycles of PCR, 7 to 20 cycles of PCR, 7 to 18 cycles of PCR, 7 to 16 cycles of PCR, 7 to 14 cycles of PCR, 7 to 12 cycles of PCR, 7 to 11 cycles of PCR, 7 to 10 cycles of PCR, 7 to 9 cycles of PCR, 8 to 20 cycles of PCR, 8 to 18 cycles of PCR, 8 to 16 cycles of PCR, 8 to 16 cycles of PCR, 8 to 14 cycles of PCR, 8 to 12 cycles of PCR, 8 to 11 cycles of PCR, 8 to 10 cycles of PCR, 8 to 9 cycles of PCR, 9 to 20 cycles of PCR, 9 to 18 cycles of PCR, 9 to 16 cycles of PCR, 9 to 16 cycles of PCR, 9 to 14 cycles of PCR, 9 to 12 cycles of PCR, 9 to 11 cycles of PCR, 9 to 10 cycles of PCR, 10 to 20 cycles of PCR, 10 to 18 cycles of PCR, 10 to 16 cycles of PCR, 10 to 16 cycles of PCR, 10 to 14 cycles of PCR, 10 to 12 cycles of PCR, 10 to 11 cycles of PCR, 11 to 20 cycles of PCR, 11 to 18 cycles of PCR, 11 to 16 cycles of PCR, 11 to 16 cycles of PCR, 11 to 14 cycles of PCR, 11 to 12 cycles of PCR, 12 to 20 cycles of PCR, 12 to 18 cycles of PCR, 12 to 16 cycles of PCR, 12 to 16 cycles of PCR, 12 to 14 cycles of PCR, 13 to 20 cycles of PCR, 13 to 18 cycles of PCR, 13 to 16 cycles of PCR, 13 to 16 cycles of PCR, 13 to 14 cycles of PCR, 14 to 20 cycles of PCR, 14 to 18 cycles of PCR, 14 to 16 cycles of PCR, 14 to 16 cycles of PCR, 16 to 20 cycles of PCR, 16 to 18 cycles of PCR, 17 to 20 cycles of PCR, or 17 to 18 cycles of PCR). In some embodiments, the amplifying of step (b) is 8 cycle of PCR or 16 cycles of PCR.

In some embodiments, in step (e), the pathogen nucleic acid in the amplified solution of step (c) is further amplified by 30, 31, 32, 33, 34, 35, 36, 37, 38, 39, 40, 41, 42, 43, 44, 45, 46, 47, 48, 49, or 50 cycles of PCR (e.g., 30 to 50 cycles of PCR, 32 to 50 cycles of PCR, 34 to 50 cycles of PCR, 36 to 50 cycles of PCR, 38 to 50 cycles of PCR, 40 to 50 cycles of PCR, 42 to 50 cycles of PCR, 44 to 50 cycles of PCR, 46 to 50 cycles of PCR, 48 to 50 cycles of PCR, 30 to 48 cycles of PCR, 32 to 48 cycles of PCR, 34 to 48 cycles of PCR, 36 to 48 cycles of PCR, 38 to 48 cycles of PCR, 40 to 48 cycles of PCR, 42 to 48 cycles of PCR, 44 to 48 cycles of PCR, 46 to 48 cycles of PCR, 30 to 46 cycles of PCR, 32 to 46 cycles of PCR, 34 to 46 cycles of PCR, 36 to 46 cycles of PCR, 38 to 46 cycles of PCR, 40 to 46 cycles of PCR, 42 to 46 cycles of PCR, 44 to 46 cycles of PCR, 30 to 44 cycles of PCR, 32 to 44 cycles of PCR, 34 to 44 cycles of PCR, 36 to 44 cycles of PCR, 38 to 44 cycles of PCR, 40 to 44 cycles of PCR, 42 to 44 cycles of PCR, 30 to 42 cycles of PCR, 32 to 42 cycles of PCR, 34 to 42 cycles of PCR, 36 to 42 cycles of PCR, 38 to 42 cycles of PCR, 40 to 42 cycles of PCR, 30 to 40 cycles of PCR, 32 to 40 cycles of PCR, 34 to 40 cycles of PCR, 36 to 40 cycles of PCR, 38 to 40 cycles of PCR, 30 to 38 cycles of PCR, 32 to 38 cycles of PCR, 34 to 38 cycles of PCR, 36 to 38 cycles of PCR, 30 to 36 cycles of PCR, 32 to 36 cycles of PCR, 34 to 36 cycles of PCR, 30 to 34 cycles of PCR, 32 to 34 cycles of PCR, 30 to 32 cycles of PCR, 35 to 50 cycles of PCR, 45 to 50 cycles of PCR, 35 to 50 cycles of PCR, or 44 to 47 cycles of PCR).

In some aspects, the biological sample is a whole blood sample.

In some aspects, the lysis agent of step (a) is an erythrocyte lysis agent.

In some aspects, the amplifying of step (b) and step (e) is in a multiplexed PCR reaction configured to amplify a plurality of target pathogen nucleic acids (e.g., 2, 3, 4, 5, 6, 7, 8, 9, 10, 11, 12, 13, 14, 15, 16, 17, 18, 19, 20, or more target pathogen nucleic acids).

In some examples, step (c) involves adding a plurality of pathogen-specific fluorescent probes (e.g., 2, 3, 4, 5, 6, 7 8, 9, 10, 11, 12, 13, 14, 15, 16, 17, 18, 19, 20, or more different pathogen-specific fluorescent probes) to the amplified solution. In one embodiment, 3 different pathogen-specific fluorescent probes are added to the amplified solution. In other embodiments, 4 to 8 different pathogen-specific fluorescent probes are added to the amplified solution. In some examples, a target nucleic acid may be detected using a plurality of fluorescent probes having the same color (e.g., 2, 3, 4, 5, or more fluorescent probes having the same color). In some examples, a target nucleic acid may be detected using 3 fluorescent probes having the same color.

In some examples, the plurality of pathogen-specific fluorescent probes comprises three or more different pathogen-specific fluorescent probes.

In some examples, the plurality of pathogen-specific fluorescent probes is between 5 and 15 probes.

In some examples, a plurality (e.g., 2, 3, 4, 5, 6, or more) of pathogen-specific fluorescent probes is used to detect a single target nucleic acid. In some embodiments, 3 pathogen-specific fluorescent probes is used to detect a single target nucleic acid.

In some examples, step (c) comprises transferring all or part of the amplified solution of step (b) to a multi-well plate containing the pathogen-specific fluorescent probe.

Any suitable multi-well plate may be used. For example, in some embodiments, the multi-well plate is a 4-well plate, a 96-well plate, or a 384-well plate. In some aspects, the multi-well plate is a 4-well plate. In some aspects, the multi-well plate is a clear plate. In some aspects, the multi-well plate is a black plate.

In some embodiments, the pathogen-specific fluorescent probe of step (c) is present in a liquid volume or is a lyophilized fluorescent probe. In some examples, the pathogen-specific fluorescent probe of step (f) is present in a liquid volume.

In some embodiments, the wells of the multi-well plate each comprise a reference dye (e.g., a reference fluorescent dye). Any suitable reference dye may be used, including any of the fluorescent dyes described herein. In some embodiments, the wells of the multi-well plate each comprise the same reference dye. In some embodiments, the wells of the multi-well plate each comprise different reference dyes.

In some embodiments, an oil is added to a well of the multi-well plate after the amplified solution of step (b) is added. In some embodiments, the oil is a mineral oil. In some embodiments, the oil is a silicone oil.

In some embodiments, the multi-well plate is sealed with a plastic seal. In some embodiments, the plastic seal covers the entire multi-well plate. In some embodiments, the plastic seal is a slit-seal.

In some embodiments, the method comprises steps (i)-(iv), and wherein the pellet of step (ii) is washed by mixing with Tris-EDTA (TE) buffer. In some embodiments, the TE buffer has a volume of about 400 μL to about 2400 μL (e.g., about 400 μL, about 500 μL, about 600 μL, about 700 μL, about 800 μL, about 900 μL, about 1000 μL, about 1100 μL, about 1200 μL, about 1300 μL, about 1400 μL, about 1500 μL, about 1600 μL, about 1700 μL, about 1800 μL, about 1900 μL, about 2000 μL, about 2100 UL, about 2200 μL, about 2300 μL, or about 2400 μL). In some embodiments, the TE buffer has a volume of about 1200 μL.

In some embodiments, the method comprises steps (i)-(iv), and wherein the volume of the buffer mixed with the pellet in step (iii) is about 100 μL to about 1000 μL (e.g., about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, or about 1000 μL). In some embodiments, the volume of the buffer mixed with the pellet in step (iii) is about 200 μL to about 500 μL. In some embodiments, the volume of the buffer mixed with the pellet in step (iii) is about 300 μL.

In some embodiments, the buffer of step (iv) is a PCR buffer that has a moderately alkaline pH at ambient temperature.

In some embodiments, the lysing of step (a) is by detergent lysis, hypotonic lysis, and/or beadbeating.

In some embodiments, the amplified solution of step (b) comprises whole blood proteins and non-target oligonucleotides.

In one embodiment, the invention provides a method for amplifying and detecting a target nucleic acid in a biological sample obtained from a subject, wherein the biological sample includes subject-derived cells or cell debris. The method including: (a) contacting a biological sample (e.g., whole blood) suspected of containing one or more pathogen cells with a lysis agent, thereby lysing cells within the biological sample; (b) centrifuging the product of step (a) to form a supernatant and a pellet; (c) discarding some or all of the supernatant of step (b) and washing the pellet once; (d) centrifuging the product of step (c) to form a supernatant and a pellet; (e) discarding some or all of the supernatant of step (d) and mixing the pellet of (d) with a buffer to form a solution containing both subject cell nucleic acid and pathogen nucleic acid; (f) amplifying the pathogen nucleic acid in the solution of step (e) by 6 to 20 cycles of PCR to form an amplified solution; (g) adding a pathogen-specific fluorescent probe to the amplified solution; (h) measuring a signal from the pathogen-specific fluorescent probe in the amplified solution of step (g) to obtain a baseline measurement; (i) further amplifying the pathogen nucleic acid in the amplified solution of step (g) by 30 to 50 cycles of PCR; (j) measuring the signal from the pathogen-specific fluorescent probe in the amplified solution of step (i); and (k) obtaining a final signal measurement by subtracting the baseline measurement from step (h) from the signal from step (j). The presence of a pathogen in the sample is detected based on the final signal measurement.

In some embodiments, the amplifying of step (f) is symmetric PCR or asymmetric PCR. In some embodiments, the amplifying of step (f) is symmetric PCR. In other embodiments, the amplifying of step (f) is asymmetric PCR.

In some embodiments, in step (f), the pathogen nucleic acid in the solution of step (e) is amplified by 6, 7, 8, 9, 10, 11, 12, 13, 14, 15, 16, 17, 18, 19, or 20 cycles of PCR (e.g., 6 to 20 cycles of PCR, 6 to 18 cycles of PCR, 6 to 16 cycles of PCR, 6 to 14 cycles of PCR, 6 to 12 cycles of PCR, 6 to 10 cycles of PCR, 6 to 8 cycles of PCR, 7 to 20 cycles of PCR, 7 to 18 cycles of PCR, 7 to 16 cycles of PCR, 7 to 14 cycles of PCR, 7 to 12 cycles of PCR, 7 to 11 cycles of PCR, 7 to 10 cycles of PCR, 7 to 9 cycles of PCR, 8 to 20 cycles of PCR, 8 to 18 cycles of PCR, 8 to 16 cycles of PCR, 8 to 16 cycles of PCR, 8 to 14 cycles of PCR, 8 to 12 cycles of PCR, 8 to 11 cycles of PCR, 8 to 10 cycles of PCR, 8 to 9 cycles of PCR, 9 to 20 cycles of PCR, 9 to 18 cycles of PCR, 9 to 16 cycles of PCR, 9 to 16 cycles of PCR, 9 to 14 cycles of PCR, 9 to 12 cycles of PCR, 9 to 11 cycles of PCR, 9 to 10 cycles of PCR, 10 to 20 cycles of PCR, 10 to 18 cycles of PCR, 10 to 16 cycles of PCR, 10 to 16 cycles of PCR, 10 to 14 cycles of PCR, 10 to 12 cycles of PCR, 10 to 11 cycles of PCR, 11 to 20 cycles of PCR, 11 to 18 cycles of PCR, 11 to 16 cycles of PCR, 11 to 16 cycles of PCR, 11 to 14 cycles of PCR, 11 to 12 cycles of PCR, 12 to 20 cycles of PCR, 12 to 18 cycles of PCR, 12 to 16 cycles of PCR, 12 to 16 cycles of PCR, 12 to 14 cycles of PCR, 13 to 20 cycles of PCR, 13 to 18 cycles of PCR, 13 to 16 cycles of PCR, 13 to 16 cycles of PCR, 13 to 14 cycles of PCR, 14 to 20 cycles of PCR, 14 to 18 cycles of PCR, 14 to 16 cycles of PCR, 14 to 16 cycles of PCR, 16 to 20 cycles of PCR, 16 to 18 cycles of PCR, 17 to 20 cycles of PCR, or 17 to 18 cycles of PCR). In some embodiments, the amplifying of step (f) is 8 cycle of PCR or 16 cycles of PCR.

In some embodiments, in step (i), the pathogen nucleic acid in the amplified solution of step (g) is further amplified by 30, 31, 32, 33, 34, 35, 36, 37, 38, 39, 40, 41, 42, 43, 44, 45, 46, 47, 48, 49, or 50 cycles of PCR (e.g., 30 to 50 cycles of PCR, 32 to 50 cycles of PCR, 34 to 50 cycles of PCR, 36 to 50 cycles of PCR, 38 to 50 cycles of PCR, 40 to 50 cycles of PCR, 42 to 50 cycles of PCR, 44 to 50 cycles of PCR, 46 to 50 cycles of PCR, 48 to 50 cycles of PCR, 30 to 48 cycles of PCR, 32 to 48 cycles of PCR, 34 to 48 cycles of PCR, 36 to 48 cycles of PCR, 38 to 48 cycles of PCR, 40 to 48 cycles of PCR, 42 to 48 cycles of PCR, 44 to 48 cycles of PCR, 46 to 48 cycles of PCR, 30 to 46 cycles of PCR, 32 to 46 cycles of PCR, 34 to 46 cycles of PCR, 36 to 46 cycles of PCR, 38 to 46 cycles of PCR, 40 to 46 cycles of PCR, 42 to 46 cycles of PCR, 44 to 46 cycles of PCR, 30 to 44 cycles of PCR, 32 to 44 cycles of PCR, 34 to 44 cycles of PCR, 36 to 44 cycles of PCR, 38 to 44 cycles of PCR, 40 to 44 cycles of PCR, 42 to 44 cycles of PCR, 30 to 42 cycles of PCR, 32 to 42 cycles of PCR, 34 to 42 cycles of PCR, 36 to 42 cycles of PCR, 38 to 42 cycles of PCR, 40 to 42 cycles of PCR, 30 to 40 cycles of PCR, 32 to 40 cycles of PCR, 34 to 40 cycles of PCR, 36 to 40 cycles of PCR, 38 to 40 cycles of PCR, 30 to 38 cycles of PCR, 32 to 38 cycles of PCR, 34 to 38 cycles of PCR, 36 to 38 cycles of PCR, 30 to 36 cycles of PCR, 32 to 36 cycles of PCR, 34 to 36 cycles of PCR, 30 to 34 cycles of PCR, 32 to 34 cycles of PCR, 30 to 32 cycles of PCR, 35 to 50 cycles of PCR, 45 to 50 cycles of PCR, 35 to 50 cycles of PCR, or 44 to 47 cycles of PCR).

In some embodiments, the amplifying of step (f) and step (i) is in a multiplexed PCR reaction configured to amplify a plurality of target pathogen nucleic acids (e.g., 2, 3, 4, 5, 6, 7, 8, 9, 10, 11, 12, 13, 14, 15, 16, 17, 18, 19, 20, or more target pathogen nucleic acids).

In some embodiments, step (g) involves adding a plurality of pathogen-specific fluorescent probes (e.g., 2, 3, 4, 5, 6, 7 8, 9, 10, 11, 12, 13, 14, 15, 16, 17, 18, 19, 20, or more different pathogen-specific fluorescent probes) to the amplified solution. In one embodiment, 3 different pathogen-specific fluorescent probes are added to the amplified solution. In other embodiments, 4 to 8 different pathogen-specific fluorescent probes are added to the amplified solution. In some examples, a target nucleic acid may be detected using a plurality of fluorescent probes having the same color (e.g., 2, 3, 4, 5, or more fluorescent probes having the same color). In some examples, a target nucleic acid may be detected using 3 fluorescent probes having the same color.

In some embodiments, the pathogen-specific fluorescent probe is a molecular beacon (e.g., a molecular beacon containing an organic dye fluorophore and a quencher). In some embodiments, the organic dye fluorophore is ATTO 425, FAM, HEX, ATTO 633, Cy5, ROX, TAMARA, Cy5.5, ALEXA 750, or a quantum dot. In some embodiments, the organic dye fluorophore is ATTO 425, FAM, HEX, ROX, ATTO 633, Cy5.5, or ALEXA 750. For example, any of the methods or devices disclosed herein may be configured to detect one or more (e.g., 1, 2, 3, 4, 5, 6, or all 7) of ATTO 425, FAM, HEX, ROX, ATTO 633, Cy5.5, or ALEXA 750. For example, the device may include a detector with filters for one or more of ATTO 425, FAM, HEX, ROX, ATTO 633, Cy5.5, or ALEXA 750. In some embodiments, the organic dye fluorophore is ATTO 425. In some embodiments, the organic dye fluorophore is FAM. In some embodiments, the organic dye fluorophore is HEX. In some embodiments, the organic dye fluorophore is ROX. In some embodiments, the organic dye fluorophore is ATTO 633. In some embodiments, the organic dye fluorophore is Cy5.5. In some embodiments, the organic dye fluorophore is ALEXA 750. In some embodiments, the quencher is Black Hole Quencher 1, Black Hole Quencher 2, Iowa Black FQ, Iowa Black RQ, a gold nanoparticle, or a silica nanoparticle.

In some embodiments, the pathogen-specific fluorescent probes are contained in wells of a multi-well plate (e.g., a 2-well, 3-well, 4-well, 5-well, 6-well, 7-well, 8-well, 10-well, 12-well, 14-well, 16-well, 18-well, 20-well, 22-well, 24-well, 48-well, 96-well, or 384-well plate). In some embodiments, the multi-well plate is a 4-well plate. In some embodiments the multi-well plate is a clear plate. In other embodiments, the multi-well plate is a black plate. In other embodiments, the wells of the multi-well plate are thin walled. In some embodiments, the wells of the multi-well plate are round bottomed.

In some embodiments, an oil (e.g., a mineral oil or a silicone oil) is added to each well of a multi-well plate to which the amplified solution of step (f) was previously added.

In some embodiments, the multi-well plate is sealed with a plastic seal prior to the amplifying of step (i). In some embodiments the plastic seal is a whole plate plastic seal. In other embodiments, the plastic seal is a slit-seal.

In some embodiments, the multi-well plate is not sealed with a plastic seal.

In some embodiments, the final concentration of the thermostable nucleic acid polymerase used in step (f) or (i) is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the denatured reaction mixture. In some embodiments, step (f) or (i) includes adding to the denatured reaction mixture at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻³ micrograms, about 2×10⁻³ micrograms, 3×10⁻³ micrograms, about 4×10⁻³ micrograms, about 5×10⁻³ micrograms, about 6×10⁻³ micrograms, about 7×10⁻³ micrograms, about 8×10⁻³ micrograms, about 9×10⁻³ micrograms, about 0.01 micrograms, about 0.02 micrograms, about 0.03 micrograms, about 0.04 micrograms, about 0.05 micrograms, or more) of a thermostable nucleic acid polymerase per microliter of denatured reaction mixture. In some embodiments, the biological sample is about 0.2 mL to about 5 mL (e.g., about 0.2 mL, about 0.3 mL, about 0.4 mL, about 0.5 mL, about 0.6 mL, about 0.7 mL, about 0.8 mL, about 0.9 mL, about 1 mL, about 1.5 mL, about 2 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, or about 10 mL). In some embodiments, the biological sample is about 0.9 mL. In some embodiments, the biological sample is whole blood, a crude blood lysate, serum, or plasma.

In other embodiments, the invention provides a method for amplifying and detecting a target nucleic acid in a whole blood sample, the method including: (a) providing a crude blood lysate produced by lysing the red blood cells in a whole blood sample from a subject, centrifuging the sample to form a supernatant and a pellet including cells, discarding some or all of the supernatant, optionally washing the pellet, and lysing the cells in the pellet; (b) adding a buffer solution including a buffering agent to the crude blood lysate to form a reaction mixture, wherein the PCR buffer has a moderately alkaline pH at ambient temperature; (c) following step (b), heating the reaction mixture to form a denatured reaction mixture; (d) adding a thermostable nucleic acid polymerase to the denatured reaction mixture; and (e) amplifying the target nucleic acid to form an amplified solution including an amplicon, e.g., as described herein (e.g., in a 2-stage PCR approach, e.g., any of the 2-stage approaches described herein).

In some embodiments of any of the preceding methods, the concentration of thermostable nucleic acid polymerase in the reaction mixture is elevated relative to the amount typically recommended by the manufacturer of the thermostable nucleic acid polymerase, e.g., by about 1-, 2-, 3-, 4-, 5-, 6-, 7-, 8-, 9-, or 10-fold, or more.

In yet other embodiments, the invention provides a method for amplifying and detecting a target nucleic acid in a whole blood sample, the method including: (a) providing a crude blood lysate produced by lysing the red blood cells in a whole blood sample from a subject, centrifuging the sample to form a supernatant and a pellet including cells, discarding some or all of the supernatant, optionally washing the pellet, and lysing the cells in the pellet; (b) adding a buffer solution including a buffering agent to the crude blood lysate to form a reaction mixture; (c) following step (b), heating the reaction mixture to form a denatured reaction mixture; (d) adding a thermostable nucleic acid polymerase to the denatured reaction mixture, wherein the final concentration of the thermostable nucleic acid polymerase is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the denatured reaction mixture; and (e) amplifying the target nucleic acid to form an amplified solution including an amplicon, e.g., as described herein (e.g., in a 2-stage PCR approach, e.g., any of the 2-stage approaches described herein).

In still other embodiments, the invention provides a method for amplifying and detecting a target nucleic acid in a whole blood sample, the method including: (a) providing a crude blood lysate produced by lysing the red blood cells in a whole blood sample from a subject, centrifuging the sample to form a supernatant and a pellet including cells, discarding some or all of the supernatant, optionally washing the pellet, and lysing the cells in the pellet; (b) adding a buffer solution including a buffering agent to the crude blood lysate to form a reaction mixture; (c) following step (b), heating the reaction mixture to form a denatured reaction mixture; (d)) adding to the denatured reaction mixture at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻³ micrograms, about 2×10⁻³ micrograms, 3×10⁻³ micrograms, about 4×10⁻³ micrograms, about 5×10⁻³ micrograms, about 6×10⁻³ micrograms, about 7×10⁻³ micrograms, about 8×10⁻³ micrograms, about 9×10⁻³ micrograms, about 0.01 micrograms, about 0.02 micrograms, about 0.03 micrograms, about 0.04 micrograms, about 0.05 micrograms, or more) of a thermostable nucleic acid polymerase per microliter of denatured reaction mixture; and; and (e) amplifying the target nucleic acid to form an amplified solution including an amplicon, e.g., as described herein (e.g., in a 2-stage PCR approach, e.g., any of the 2-stage approaches described herein).

In some embodiments, the invention provides a method for amplifying and detecting a target nucleic acid in a whole blood sample, the method including one or more of the following steps: (a) providing a crude blood lysate produced by lysing the red blood cells in a whole blood sample from a subject, centrifuging the sample to form a supernatant and a pellet including cells, discarding some or all of the supernatant, optionally washing the pellet, and lysing the cells in the pellet; (b) adding to the crude blood lysate a buffer solution including a buffering agent to form a reaction mixture, wherein the buffer solution has a moderately alkaline pH at ambient temperature; (c) following step (b), heating the reaction mixture to form a denatured reaction mixture; (d) adding a thermostable nucleic acid polymerase to the denatured reaction mixture, wherein the final concentration of the thermostable nucleic acid polymerase is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the denatured reaction mixture; and (e) amplifying the target nucleic acid to form an amplified solution including an amplicon, e.g., as described herein (e.g., in a 2-stage PCR approach, e.g., any of the 2-stage approaches described herein).

In another example, in some embodiments, the invention provides a method for amplifying and detecting a target nucleic acid in a whole blood sample, the method including one or more of the following steps: (a) providing a crude blood lysate produced by lysing the red blood cells in a whole blood sample from a subject, centrifuging the sample to form a supernatant and a pellet including cells, discarding some or all of the supernatant, optionally washing the pellet, and lysing the cells in the pellet; (b) adding to the crude blood lysate a buffer solution including a buffering agent to form a reaction mixture, wherein the buffer solution has a moderately alkaline pH at ambient temperature; (c) following step (b), heating the reaction mixture to form a denatured reaction mixture; (d) adding to the denatured reaction mixture at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻³ micrograms, about 2×10⁻³ micrograms, 3×10⁻³ micrograms, about 4×10⁻³ micrograms, about 5×10⁻³ micrograms, about 6×10⁻³ micrograms, about 7×10⁻³ micrograms, about 8×10⁻³ micrograms, about 9×10⁻³ micrograms, about 0.01 micrograms, about 0.02 micrograms, about 0.03 micrograms, about 0.04 micrograms, about 0.05 micrograms, or more) of a thermostable nucleic acid polymerase per microliter of denatured reaction mixture; and (e) amplifying the target nucleic acid to form an amplified solution including an amplicon, e.g., as described herein (e.g., in a 2-stage PCR approach, e.g., any of the 2-stage approaches described herein).

In some embodiments of any of the preceding methods, the final concentration of the thermostable nucleic acid polymerase may range from about 0.01 units to about 1 unit (e.g., about 0.01 units to about 1 unit, about 0.01 units to about 0.9 units, about 0.01 units to about 0.8 units, about 0.01 units to about 0.7 units, about 0.01 units to about 0.6 units, about 0.01 units to about 0.5 units, about 0.01 units to about 0.4 units, about 0.01 units to about 0.3 units, about 0.01 units to about 0.25 units, about 0.01 units to about 0.2 units, about 0.01 units to about 0.1 unit, about 0.02 units to about 1 unit, about 0.02 units to about 0.9 units, about 0.02 units to about 0.8 units, about 0.02 units to about 0.7 units, about 0.02 units to about 0.6 units, about 0.02 units to about 0.5 units, about 0.02 units to about 0.4 units, about 0.02 units to about 0.3 units, about 0.02 units to about 0.25 units, about 0.02 units to about 0.2 units, about 0.02 units to about 0.1 units, about 0.04 units to about 1 unit, about 0.04 units to about 0.9 units, about 0.04 units to about 0.8 units, about 0.04 units to about 0.7 units, about 0.04 units to about 0.6 units, about 0.04 units to about 0.5 units, about 0.04 units to about 0.4 units, about 0.04 units to about 0.3 units, about 0.04 units to about 0.25 units, about 0.04 units to about 0.2 units, about 0.04 units to about 0.1 units, about 0.06 units to about 1 unit, about 0.06 units to about 0.9 units, about 0.06 units to about 0.8 units, about 0.06 units to about 0.7 units, about 0.06 units to about 0.6 units, about 0.06 units to about 0.5 units, about 0.06 units to about 0.4 units, about 0.06 units to about 0.3 units, about 0.06 units to about 0.25 units, about 0.06 units to about 0.2 units, about 0.06 units to about 0.1 units, about 0.08 units to about 1 unit, about 0.08 units to about 0.9 units, about 0.08 units to about 0.8 units, about 0.08 units to about 0.7 units, about 0.08 units to about 0.6 units, about 0.08 units to about 0.5 units, about 0.08 units to about 0.4 units, about 0.08 units to about 0.3 units, about 0.08 units to about 0.25 units, about 0.08 units to about 0.2 units, about 0.08 units to about 0.1 units, about 0.1 units to about 1 unit, about 0.1 units to about 0.9 units, about 0.1 units to about 0.8 units, about 0.1 units to about 0.7 units, about 0.1 units to about 0.6 units, about 0.1 units to about 0.5 units, about 0.1 units to about 0.4 units, about 0.1 units to about 0.3 units, about 0.1 units to about 0.25 units, about 0.1 units to about 0.2 units, about 0.2 units to about 1 unit, about 0.2 units to about 0.9 units, about 0.2 units to about 0.8 units, about 0.2 units to about 0.7 units, about 0.2 units to about 0.6 units, about 0.2 units to about 0.5 units, about 0.2 units to about 0.4 units, about 0.2 units to about 0.3 units, about 0.2 units to about 0.25 units, about 0.3 units to about 1 unit, about 0.3 units to about 0.9 units, about 0.3 units to about 0.8 units, about 0.3 units to about 0.7 units, about 0.3 units to about 0.6 units, about 0.3 units to about 0.5 units, about 0.3 units to about 0.4 units, about 0.4 units to about 1 unit, about 0.4 units to about 0.9 units, about 0.4 units to about 0.8 units, about 0.4 units to about 0.7 units, about 0.4 units to about 0.6 units, about 0.4 units to about 0.5 units, about 0.5 units to about 1 unit, about 0.5 units to about 0.9 units, about 0.5 units to about 0.8 units, about 0.5 units to about 0.7 units, about 0.5 units to about 0.6 units, about 0.6 units to about 1 unit, about 0.6 units to about 0.9 units, about 0.6 units to about 0.8 units, about 0.6 units to about 0.7 units, about 0.6 units to about 0.6 units, about 0.7 units to about 1 unit, about 0.7 units to about 0.9 units, about 0.7 units to about 0.8 units, about 0.8 units to about 1 unit, or about 0.8 units to about 0.9 units) per microliter of the denatured reaction mixture.

In some embodiments of any of the preceding methods, step (d) may include adding to the denatured reaction mixture from about 1×10⁻⁵ micrograms to about 0.05 micrograms (e.g., about 1×10⁻⁵ micrograms to about 0.05 micrograms, about 1×10⁻⁵ micrograms to about 0.025 micrograms, about 1×10⁻⁵ micrograms to about 0.01 micrograms, about 1×10⁻⁵ micrograms to about 0.0075 micrograms, about 1×10⁻⁵ micrograms to about 0.005 micrograms, about 1×10⁻⁵ micrograms to about 0.0025 micrograms, about 1×10⁻⁵ micrograms to about 0.001 micrograms, about 1×10⁻⁵ micrograms to about 1×10⁻⁴ micrograms, about 2×10⁻⁵ micrograms to about 0.05 micrograms, about 2×10⁻⁵ micrograms to about 0.025 micrograms, about 2×10⁻⁵ micrograms to about 0.01 micrograms, about 2×10⁻⁵ micrograms to about 0.0075 micrograms, about 2×10⁻⁵ micrograms to about 0.005 micrograms, about 2×10⁻⁵ micrograms to about 0.0025 micrograms, about 2×10⁻⁵ micrograms to about 0.001 micrograms, about 2×10⁻⁵ micrograms to about 1×10⁻⁴ micrograms, about 2.4×10⁻⁵ micrograms to about 0.05 micrograms, about 2.4×10⁻⁵ micrograms to about 0.025 micrograms, about 2.4×10⁻⁵ micrograms to about 0.01 micrograms, about 2.4×10⁻⁵ micrograms to about 0.0075 micrograms, about 2.4×10⁻⁵ micrograms to about 0.005 micrograms, about 2.4×10⁻⁵ micrograms to about 0.0025 micrograms, about 2.4×10⁻⁵ micrograms to about 0.001 micrograms, about 2.4×10⁻⁵ micrograms to about 1×10⁻⁴ micrograms, about 5×10⁻⁵ micrograms to about 0.05 micrograms, about 5×10⁻⁵ micrograms to about 0.025 micrograms, about 5×10⁻⁵ micrograms to about 0.01 micrograms, about 5×10⁻⁵ micrograms to about 0.0075 micrograms, about 5×10⁻⁵ micrograms to about 0.005 micrograms, about 5×10⁻⁵ micrograms to about 0.0025 micrograms, about 5×10⁻⁵ micrograms to about 0.001 micrograms, about 5×10⁻⁵ micrograms to about 1×10⁻⁴ micrograms, about 8×10⁻⁵ micrograms to about 0.05 micrograms, about 8×10⁻⁵ micrograms to about 0.025 micrograms, about 8×10⁻⁵ micrograms to about 0.01 micrograms, about 8×10⁻⁵ micrograms to about 0.0075 micrograms, about 8×10⁻⁵ micrograms to about 0.005 micrograms, about 8×10⁻⁵ micrograms to about 0.0025 micrograms, about 8×10⁻⁵ micrograms to about 0.001 micrograms, about 8×10⁻⁵ micrograms to about 1×10⁻⁴ micrograms, about 1×10⁻⁴ micrograms to about 0.05 micrograms, about 1×10⁻⁴ micrograms to about 0.025 micrograms, about 1×10⁻⁴ micrograms to about 0.01 micrograms, about 1×10⁻⁴ micrograms to about 0.0075 micrograms, about 1×10⁻⁴ micrograms to about 0.005 micrograms, about 1×10⁻⁴ micrograms to about 0.0025 micrograms, about 1×10⁻⁴ micrograms to about 0.001 micrograms, about 5×10⁻⁴ micrograms to about 0.05 micrograms, about 5×10⁻⁴ micrograms to about 0.025 micrograms, about 5×10⁻⁴ micrograms to about 0.01 micrograms, about 5×10⁻⁴ micrograms to about 0.0075 micrograms, about 5×10⁻⁴ micrograms to about 0.005 micrograms, about 5×10⁻⁴ micrograms to about 0.0025 micrograms, about 5×10⁻⁴ micrograms to about 0.001 micrograms, about 1×10⁻³ micrograms to about 0.05 micrograms, about 1×10⁻³ micrograms to about 0.025 micrograms, about 1×10⁻³ micrograms to about 0.01 micrograms, about 1×10⁻³ micrograms to about 0.0075 micrograms, about 1×10⁻³ micrograms to about 0.005 micrograms, or about 1×10⁻³ micrograms to about 0.0025 micrograms) of a thermostable nucleic acid polymerase per microliter of denatured reaction mixture. In some embodiments of any of the preceding methods, step (c) may further include centrifuging the denatured reaction mixture prior to step (d). In some embodiments of any of the preceding methods, step (c) may include heating the reaction mixture to greater than about 55° C., e.g., 55° C., 60° C., 65° C., 70° C., 75° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

In some embodiments of any of the preceding methods, the method further includes adding (i) deoxynucleotide triphosphates (dNTPs), (ii) magnesium, (iii) one or more forward primers (e.g., 1, 2, 3, 4, 5, 6, 7, 8, 9, 10, 11, 12, 13, 14, 15, 16, 17, 18, 19, 20, or more forward primers), and/or (iv) one or more reverse primers (e.g., 1, 2, 3, 4, 5, 6, 7, 8, 9, 10, 11, 12, 13, 14, 15, 16, 17, 18, 19, 20, or more reverse primers) during any amplification step.

In some embodiments of any of the preceding methods, the whole blood sample is about 0.2 mL to about 10 mL (e.g., about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, or about 10 mL).

In some embodiments of any of the preceding methods, the whole blood sample is about 0.2 mL to about 10 mL, about 0.2 mL to about 9.5 mL, about 0.2 mL to about 9 mL, about 0.2 mL to about 8.5 mL, about 0.2 mL to about 8 mL, about 0.2 mL to about 7.5 mL, about 0.2 mL to about 7 mL, about 0.2 mL to about 6.5 mL, about 0.2 mL to about 6 mL, about 0.2 mL to about 5.5 mL, about 0.2 mL to about 5 mL, about 0.2 mL to about 4.5 mL, about 0.2 mL to about 4 mL, about 0.2 mL to about 3.5 mL, about 0.2 mL to about 3 mL, about 0.2 mL to about 2.5 mL, about 0.2 mL to about 2 mL, about 0.2 mL to about 1.5 mL, about 0.2 mL to about 1 mL, about 0.2 mL to about 0.5 mL, about 0.5 mL to about 10 mL, about 0.5 mL to about 9.5 mL, about 0.5 mL to about 9 mL, about 0.5 mL to about 8.5 mL, about 0.5 mL to about 8 mL, about 0.5 mL to about 7.5 mL, about 0.5 mL to about 7 mL, about 0.5 mL to about 6.5 mL, about 0.5 mL to about 6 mL, about 0.5 mL to about 5.5 mL, about 0.5 mL to about 5 mL, about 0.5 mL to about 4.5 mL, about 0.5 mL to about 4 mL, about 0.5 mL to about 3.5 mL, about 0.5 mL to about 3 mL, about 0.5 mL to about 2.5 mL, about 0.5 mL to about 2 mL, about 0.5 mL to about 1.5 mL, about 0.5 mL to about 1 mL, about 1 mL to about 10 mL, about 1 mL to about 9.5 mL, about 1 mL to about 9 mL, about 1 mL to about 8.5 mL, about 1 mL to about 8 mL, about 1 mL to about 7.5 mL, about 1 mL to about 7 mL, about 1 mL to about 6.5 mL, about 1 mL to about 6 mL, about 1 mL to about 5.5 mL, about 1 mL to about 5 mL, about 1 mL to about 4.5 mL, about 1 mL to about 4 mL, about 1 mL to about 3.5 mL, about 1 mL to about 3 mL, about 1 mL to about 2.5 mL, about 1 mL to about 2 mL, about 1 mL to about 1.5 mL, about 2 mL to about 10 mL, about 2 mL to about 9.5 mL, about 2 mL to about 9 mL, about 2 mL to about 8.5 mL, about 2 mL to about 8 mL, about 2 mL to about 7.5 mL, about 2 mL to about 7 mL, about 2 mL to about 6.5 mL, about 2 mL to about 6 mL, about 2 mL to about 5.5 mL, about 2 mL to about 5 mL, about 2 mL to about 4.5 mL, about 2 mL to about 4 mL, about 2 mL to about 3.5 mL, about 2 mL to about 3 mL, about 2 mL to about 2.5 mL, about 3 mL to about 10 mL, about 3 mL to about 9.5 mL, about 3 mL to about 9 mL, about 3 mL to about 8.5 mL, about 3 mL to about 8 mL, about 3 mL to about 7.5 mL, about 3 mL to about 7 mL, about 3 mL to about 6.5 mL, about 3 mL to about 6 mL, about 3 mL to about 5.5 mL, about 3 mL to about 5 mL, about 3 mL to about 4.5 mL, about 3 mL to about 4 mL, about 3 mL to about 3.5 mL, about 4 mL to about 10 mL, about 4 mL to about 9.5 mL, about 4 mL to about 9 mL, about 4 mL to about 8.5 mL, about 4 mL to about 8 mL, about 4 mL to about 7.5 mL, about 4 mL to about 7 mL, about 4 mL to about 6.5 mL, about 4 mL to about 6 mL, about 4 mL to about 5.5 mL, about 4 mL to about 5 mL, about 4 mL to about 4.5 mL, about 5 mL to about 10 mL, about 5 mL to about 9.5 mL, about 5 mL to about 9 mL, about 5 mL to about 8.5 mL, about 5 mL to about 8 mL, about 5 mL to about 7.5 mL, about 5 mL to about 7 mL, about 5 mL to about 6.5 mL, about 5 mL to about 6 mL, about 5 mL to about 5.5 mL, about 6 mL to about 10 mL, about 6 mL to about 9.5 mL, about 6 mL to about 9 mL, about 6 mL to about 8.5 mL, about 6 mL to about 8 mL, about 6 mL to about 7.5 mL, about 6 mL to about 7 mL, about 6 mL to about 6.5 mL, about 7 mL to about 10 mL, about 7 mL to about 9.5 mL, about 7 mL to about 9 mL, about 7 mL to about 8.5 mL, about 7 mL to about 8 mL, about 7 mL to about 7.5 mL, about 8 mL to about 10 mL, about 8 mL to about 9.5 mL, about 8 mL to about 9 mL, about 8 mL to about 8.5 mL, about 9 mL to about 10 mL, or about 9 mL to about 9.5 mL. In some embodiments of any of the preceding methods, the whole blood sample is about 6 mL.

The invention allows use of a concentrated crude blood lysate prepared from a larger volume of whole blood. In some embodiments, a crude blood lysate produced from a whole blood sample of about 0.2 mL to about 10 mL has a volume of about 10 μL to about 1000 μL (e.g., about 10 μL, about 20 μL about 30 μL, about 40 μL, about 50 μL, about 60 μL, about 70 μL, about 80 μL, about 90 μL, about 100 μL, about 125 μL, about 150 μL, about 175 μL, about 200 μL, about 225 μL, about 250 μL, about 275 μL, about 300 μL, about 325 μL, about 350 μL, about 375 μL, about 400 μL, about 425 μL, about 450 μL, about 475 μL, about 500 μL, about 525 μL, about 550 μL, about 600 μL, about 625 μL, about 650 μL, about 675 μL, about 700 μL, about 725 μL, about 750 μL, about 775 μL, about 800 μL, about 825 μL, about 850 μL, about 875 μL, about 900 μL, about 925 μL, about 950 μL, about 975 μL, or about 1000 μL). In some embodiments, the crude blood lysate produced from the whole blood sample has a volume of about 25 μL to about 200 μL. In some embodiments, the crude blood lysate produced from the whole blood sample has a volume of about 50 μL.

In some embodiments, the crude blood lysate is concentrated at least 2-fold, 3-fold, 4-fold, 5-fold, 6-fold, 7-fold, 8-fold, 9-fold, 10-fold, or more compared to the whole blood sample.

In some embodiments, the reaction mixture for amplification contains about 20% to about 60% crude blood lysate (e.g., about 20%, about 25%, about 30%, about 35%, about 40%, about 45%, about 50%, about 55%, or about 60% crude blood lysate).

In some embodiments of any of the preceding methods, the denatured reaction mixture has a volume ranging from about 0.1 μL to about 250 μL or more, e.g., about 1 μL, about 10 μL, about 20 μL, about 30 μL, about 40 μL, about 50 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, or more. In some embodiments, the volume of the denatured reaction mixture is about 100 μL.

In another example, in some embodiments, the invention provides a method for amplifying and detecting a target nucleic acid in a sample including unprocessed whole blood, the method including: (a) providing a mixture including a buffer solution including a buffering agent, dNTPs, magnesium, a forward primer, a reverse primer, and a thermostable nucleic acid polymerase, wherein the buffer solution has a moderately alkaline pH at ambient temperature, and wherein the final concentration of the thermostable nucleic acid polymerase is at least about 0.01 units (e.g., about 0.01 units, about 0.02 units, about 0.03 units, about 0.04 units, about 0.05 units, about 0.06 units, about 0.07 units, about 0.08 units, about 0.09 units, about 0.10 units, about 0.15 units about 0.2 units, about 0.25 units, about 0.3 units, about 0.35 units, about 0.4 units, about 0.45 units, about 0.5 units, about 0.6 units, about 0.65 units, about 0.7 units, about 0.8 units, about 0.9 units, about 1 unit, or more) per microliter of the mixture; (b) adding to the mixture a portion of a whole blood sample obtained from a subject to form a reaction mixture; and (c) amplifying the target nucleic acid to form an amplified solution including an amplicon, e.g., as described herein (e.g., in a 2-stage PCR approach, e.g., any of the 2-stage approaches described herein). In some embodiments, the reaction mixture contains from about 1% to about 70% (v/v) whole blood, e.g., about 1%, about 2%, about 3%, about 4%, about 5%, about 10%, about 15%, about 20%, about 25%, about 30%, about 35%, about 40%, about 45%, about 50%, about 55%, about 60%, about 65%, or about 70% (v/v) whole blood).

In a still further example, in some embodiments, the invention provides a method for amplifying and detecting a target nucleic acid in a sample including whole blood, the method including: (a) providing a mixture, wherein the mixture includes a buffer solution including a buffering agent, dNTPs, magnesium, a forward primer, a reverse primer, and a thermostable nucleic acid polymerase, wherein the buffer solution has a moderately alkaline pH at ambient temperature, and wherein the mixture contains about at least about 1×10⁻⁵ micrograms (e.g., about 1×10⁻⁵ micrograms, about 1.5×10⁻⁵ micrograms, about 2×10⁻⁵ micrograms, about 2.4×10⁻⁵ micrograms, about 2.5×10⁻⁵ micrograms, about 3×10⁻⁵ micrograms, about 4×10⁻⁵ micrograms, about 5×10⁻⁵ micrograms, about 6×10⁻⁵ micrograms, about 7×10⁻⁵ micrograms, about 8×10⁻⁵ micrograms, about 9×10⁻⁵ micrograms, about 1×10⁻⁴ micrograms, about 2×10⁻⁴ micrograms, about 3×10⁻⁴ micrograms, about 4×10⁻⁴ micrograms, about 5×10⁻⁴ micrograms, about 6×10⁻⁴ micrograms, about 7×10⁻⁴ micrograms, about 8×10⁻⁴ micrograms, about 9×10⁻⁴ micrograms, about 1×10⁻³ micrograms, about 2×10⁻³ micrograms, 3×10⁻³ micrograms, about 4×10⁻³ micrograms, about 5×10⁻³ micrograms, about 6×10⁻³ micrograms, about 7×10⁻³ micrograms, about 8×10⁻³ micrograms, about 9×10⁻³ micrograms, about 0.01 micrograms, about 0.02 micrograms, about 0.03 micrograms, about 0.04 micrograms, about 0.05 micrograms, or more) of the thermostable nucleic acid polymerase per microliter of the mixture of the thermostable nucleic acid polymerase; (b) adding to the mixture a portion of a whole blood sample obtained from a subject to form a reaction mixture; and (c) amplifying the target nucleic acid to form an amplified solution including an amplicon, e.g., as described herein (e.g., in a 2-stage PCR approach, e.g., any of the 2-stage approaches described herein). In some embodiments, the reaction mixture contains from about 1% to about 70% (v/v) whole blood, e.g., about 1%, about 2%, about 3%, about 4%, about 5%, about 10%, about 15%, about 20%, about 25%, about 30%, about 35%, about 40%, about 45%, about 50%, about 55%, about 60%, about 65%, or about 70% (v/v) whole blood).

In some embodiments of any of the preceding methods, the final concentration of the thermostable nucleic acid polymerase may range from about 0.01 units to about 1 unit (e.g., about 0.01 units to about 1 unit, about 0.01 units to about 0.9 units, about 0.01 units to about 0.8 units, about 0.01 units to about 0.7 units, about 0.01 units to about 0.6 units, about 0.01 units to about 0.5 units, about 0.01 units to about 0.4 units, about 0.01 units to about 0.3 units, about 0.01 units to about 0.25 units, about 0.01 units to about 0.2 units, about 0.01 units to about 0.1 unit, about 0.02 units to about 1 unit, about 0.02 units to about 0.9 units, about 0.02 units to about 0.8 units, about 0.02 units to about 0.7 units, about 0.02 units to about 0.6 units, about 0.02 units to about 0.5 units, about 0.02 units to about 0.4 units, about 0.02 units to about 0.3 units, about 0.02 units to about 0.25 units, about 0.02 units to about 0.2 units, about 0.02 units to about 0.1 units, about 0.04 units to about 1 unit, about 0.04 unit to about 0.9 units, about 0.04 units to about 0.8 units, about 0.04 units to about 0.7 units, about 0.04 units to about 0.6 units, about 0.04 units to about 0.5 units, about 0.04 units to about 0.4 units, about 0.04 units to about 0.3 units, about 0.04 units to about 0.25 units, about 0.04 units to about 0.2 units, about 0.04 units to about 0.1 units, about 0.06 units to about 1 unit, about 0.06 units to about 0.9 units, about 0.06 units to about 0.8 units, about 0.06 units to about 0.7 units, about 0.06 units to about 0.6 units, about 0.06 units to about 0.5 units, about 0.06 units to about 0.4 units, about 0.06 units to about 0.3 units, about 0.06 units to about 0.25 units, about 0.06 units to about 0.2 units, about 0.06 units to about 0.1 units, about 0.08 units to about 1 unit, about 0.08 units to about 0.9 units, about 0.08 units to about 0.8 units, about 0.08 units to about 0.7 units, about 0.08 units to about 0.6 units, about 0.08 units to about 0.5 units, about 0.08 units to about 0.4 units, about 0.08 units to about 0.3 units, about 0.08 units to about 0.25 units, about 0.08 units to about 0.2 units, about 0.08 units to about 0.1 units, about 0.1 units to about 1 unit, about 0.1 units to about 0.9 units, about 0.1 units to about 0.8 units, about 0.1 units to about 0.7 units, about 0.1 units to about 0.6 units, about 0.1 units to about 0.5 units, about 0.1 units to about 0.4 units, about 0.1 units to about 0.3 units, about 0.1 units to about 0.25 units, about 0.1 units to about 0.2 units, about 0.2 units to about 1 unit, about 0.2 units to about 0.9 units, about 0.2 units to about 0.8 units, about 0.2 units to about 0.7 units, about 0.2 units to about 0.6 units, about 0.2 units to about 0.5 units, about 0.2 units to about 0.4 units, about 0.2 units to about 0.3 units, about 0.2 units to about 0.25 units, about 0.3 units to about 1 unit, about 0.3 units to about 0.9 units, about 0.3 units to about 0.8 units, about 0.3 units to about 0.7 units, about 0.3 units to about 0.6 units, about 0.3 units to about 0.5 units, about 0.3 units to about 0.4 units, about 0.4 units to about 1 unit, about 0.4 units to about 0.9 units, about 0.4 units to about 0.8 units, about 0.4 units to about 0.7 units, about 0.4 units to about 0.6 units, about 0.4 units to about 0.5 units, about 0.5 units to about 1 unit, about 0.5 units to about 0.9 units, about 0.5 units to about 0.8 units, about 0.5 units to about 0.7 units, about 0.5 units to about 0.6 units, about 0.6 units to about 1 unit, about 0.6 units to about 0.9 units, about 0.6 units to about 0.8 units, about 0.6 units to about 0.7 units, about 0.6 units to about 0.6 units, about 0.7 units to about 1 unit, about 0.7 units to about 0.9 units, about 0.7 units to about 0.8 units, about 0.8 units to about 1 unit, or about 0.8 units to about 0.9 units) per microliter of the mixture.

In some embodiments of any of the preceding methods, the mixture includes from about 1×10⁻⁵ micrograms to about 0.05 micrograms (e.g., about 1×10⁻⁵ micrograms to about 0.05 micrograms, about 1×10⁻⁵ micrograms to about 0.025 micrograms, about 1×10⁻⁵ micrograms to about 0.01 micrograms, about 1×10⁻⁵ micrograms to about 0.0075 micrograms, about 1×10⁻⁵ micrograms to about 0.005 micrograms, about 1×10⁻⁵ micrograms to about 0.0025 micrograms, about 1×10⁻⁵ micrograms to about 0.001 micrograms, about 1×10⁻⁵ micrograms to about 1×10⁻⁴ micrograms, about 2×10⁻⁵ micrograms to about 0.05 micrograms, about 2×10⁻⁵ micrograms to about 0.025 micrograms, about 2×10⁻⁵ micrograms to about 0.01 micrograms, about 2×10⁻⁵ micrograms to about 0.0075 micrograms, about 2×10⁻⁵ micrograms to about 0.005 micrograms, about 2×10⁻⁵ micrograms to about 0.0025 micrograms, about 2×10⁻⁵ micrograms to about 0.001 micrograms, about 2×10⁻⁵ micrograms to about 1×10⁻⁴ micrograms, about 2.4×10⁻⁵ micrograms to about 0.05 micrograms, about 2.4×10⁻⁵ micrograms to about 0.025 micrograms, about 2.4×10⁻⁵ micrograms to about 0.01 micrograms, about 2.4×10⁻⁵ micrograms to about 0.0075 micrograms, about 2.4×10⁻⁵ micrograms to about 0.005 micrograms, about 2.4×10⁻⁵ micrograms to about 0.0025 micrograms, about 2.4×10⁻⁵ micrograms to about 0.001 micrograms, about 2.4×10⁻⁵ micrograms to about 1×10⁻⁴ micrograms, about 5×10⁻⁵ micrograms to about 0.05 micrograms, about 5×10⁻⁵ micrograms to about 0.025 micrograms, about 5×10⁻⁵ micrograms to about 0.01 micrograms, about 5×10⁻⁵ micrograms to about 0.0075 micrograms, about 5×10⁻⁵ micrograms to about 0.005 micrograms, about 5×10⁻⁵ micrograms to about 0.0025 micrograms, about 5×10⁻⁵ micrograms to about 0.001 micrograms, about 5×10⁻⁵ micrograms to about 1×10⁻⁴ micrograms, about 8×10⁻⁵ micrograms to about 0.05 micrograms, about 8×10⁻⁵ micrograms to about 0.025 micrograms, about 8×10⁻⁵ micrograms to about 0.01 micrograms, about 8×10⁻⁵ micrograms to about 0.0075 micrograms, about 8×10⁻⁵ micrograms to about 0.005 micrograms, about 8×10⁻⁵ micrograms to about 0.0025 micrograms, about 8×10⁻⁵ micrograms to about 0.001 micrograms, about 8×10⁻⁵ micrograms to about 1×10⁻⁴ micrograms, about 1×10⁻⁴ micrograms to about 0.05 micrograms, about 1×10⁻⁴ micrograms to about 0.025 micrograms, about 1×10⁻⁴ micrograms to about 0.01 micrograms, about 1×10⁻⁴ micrograms to about 0.0075 micrograms, about 1×10⁻⁴ micrograms to about 0.005 micrograms, about 1×10⁻⁴ micrograms to about 0.0025 micrograms, about 1×10⁻⁴ micrograms to about 0.001 micrograms, about 5×10⁻⁴ micrograms to about 0.05 micrograms, about 5×10⁻⁴ micrograms to about 0.025 micrograms, about 5×10⁻⁴ micrograms to about 0.01 micrograms, about 5×10⁻⁴ micrograms to about 0.0075 micrograms, about 5×10⁻⁴ micrograms to about 0.005 micrograms, about 5×10⁻⁴ micrograms to about 0.0025 micrograms, about 5×10⁻⁴ micrograms to about 0.001 micrograms, about 1×10⁻³ micrograms to about 0.05 micrograms, about 1×10⁻³ micrograms to about 0.025 micrograms, about 1×10⁻³ micrograms to about 0.01 micrograms, about 1×10⁻³ micrograms to about 0.0075 micrograms, about 1×10⁻³ micrograms to about 0.005 micrograms, or about 1×10⁻³ micrograms to about 0.0025 micrograms) of the thermostable nucleic acid polymerase per microliter of the mixture.

In some embodiments of any of the preceding methods, the mixture has a volume ranging from about 0.1 μL to about 250 μL or more, e.g., about 1 μL, about 10 μL, about 20 μL, about 30 μL, about 40 μL, about 50 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, or more. In some embodiments, the volume of the mixture is about 100 μL.

In some embodiments of any of the preceding methods, the moderately alkaline pH at ambient temperature is from about pH 7.1 to about pH 11.5 or higher (e.g., about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, about pH 10.0, about pH 10.1, about pH 10.2, about pH 10.3, about pH 10.4, about pH 10.5, about pH 10.6, about pH 10.7, about pH 10.8, about pH 10.9, about pH 11, about pH 11.1, about pH 11.2, about pH 11.3, about pH 11.3, about pH 11.4, about pH 11.5, or higher. In some embodiments, the moderately alkaline pH at ambient temperature is from about pH 7.1 to about pH 11.5, about pH 7.1 to about pH 11.0, about pH 7.1 to about pH 10.5, about pH 7.1 to about pH 10.0, about pH 7.1 to about pH 9.5, about pH 7.1 to about pH 9.0, about pH 7.1 to about pH 8.5, about pH 7.1 to about pH 8, about pH 7.1 to about pH 7.5, about pH 7.5 to about pH 11.5, about pH 7.5 to about pH 11.0, about pH 7.5 to about pH 10.5, about pH 7.5 to about pH 10.0, about pH 7.5 to about pH 9.5, about pH 7.5 to about pH 9.0, about pH 7.5 to about pH 8.5, about pH 7.5 to about pH 8.0, about pH 8.0 to about pH 11.5, about pH 8.0 to about pH 11.0, about pH 8.0 to about pH 10.5, about pH 8.0 to about pH 10.0, about pH 8.0 to about pH 9.5, about pH 8.0 to about pH 9.0, about pH 8.0 to about pH 9.0, about pH 8.0 to about pH 8.5, about pH 8.5 to about pH 11.5, about pH 8.5 to about pH 11.0, about pH 8.5 to about pH 10.0, about pH 8.5 to about pH 9.5, about pH 8.5 to about pH 9.0, about pH 9.0 to about pH 11.5, about pH 9.0 to about pH 11.0, about pH 9.0 to about pH 10.5, about pH 9.0 to about pH 10.0, about pH 9.0 to about pH 9.5, about pH 9.5 to about pH 11.5, about pH 9.5 to about pH 11.0, about pH 9.5 to about pH 10.5, or about pH 9.5 to about pH 10.0. In some embodiments, the moderately alkaline pH at ambient temperature is about pH 8.7. In some embodiments, ambient temperature is about 25° C. (e.g., about 20° C., about 21° C., about 22° C., about 23° C., about 24° C., about 25° C., about 26° C., about 27° C., about 28° C., about 29° C., about 30° C.).

In some embodiments of any of the preceding methods, the pH of the buffer solution remains approximately at or above a neutral pH at 95° C. In some embodiments, the pH of the buffer solution is about pH 6.5 to about pH 10 (e.g., about pH 6.5, about pH 6.6, about pH 6.7, about pH 6.8, about pH 6.9, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, or about pH 10.0) at 95° C. For example, in some embodiments, the pH of the buffer solution at 95° C. is from about pH 6.5 to about pH 10.0, about pH 6.5 to about pH 9.5, about pH 6.5 to about pH 9.0, about pH 6.5 to about pH 8.5, about pH 6.5 to about pH 8.0, about pH 6.5 to about pH 7.5, about pH 7.0 to about pH 10.0, about pH 7.0 to about pH 9.5, about pH 7.0 to about pH 9.0, about pH 7.0 to about pH 8.5, about pH 7.0 to about pH 8.0, about pH 7.0 to about pH 7.5, about pH 7.5 to about pH 10.0, about pH 7.5 to about pH 9.5, about pH 7.5 to about pH 9.0, about pH 7.5 to about pH 8.5, about pH 7.5 to about pH 8.0, about pH 8.0 to about pH 10.0, about pH 8.0 to about pH 9.5, about pH 8.0 to about pH 9.0, about pH 8.0 to about pH 8.5, about pH 8.5 to about pH 10.0, about pH 8.5 to about pH 9.5, about pH 8.5 to about pH 9.0, about pH 9.0 to about pH 10.0, or about pH 9.5 to about pH 10.0.

Any suitable buffering agent may be used in the methods of the invention. For example, in some embodiments, any buffer with a pKa ranging from about 7.0 to about 9.2 (e.g., about 7.0 to about 7.6; from about 7.6 to about 8.2; or about 8.2 to about 9.2) may be used. Exemplary buffering agents with a pKa ranging from about 7.0 to about 7.6 include but are not limited to: MOPS, BES, phosphoric acid, TES, HEPES, and DIPSO. Exemplary buffering agents with a pKa ranging from about 7.6 to about 8.2 include but are not limited to: TAPSO, TEA, n-ethylmorpholine, POPSO, EPPS, HEPPSO, Tris, and Tricine. Exemplary buffering agents with a pKa ranging from about 8.2 to about 9.2 include but are not limited to: glycylglycine, Bicine, TAPS, morpholine, n-methyldiethanolamine, AMPD (2-amino-2-methyl-1,3-propanediol), diethanolamine, and AMPSO. In some embodiments, a buffering agent with a pKa greater than 9.2 may be used. Exemplary buffering agents with a pKa greater than 9.2 include but are not limited to boric acid, CHES, glycine, CAPSO, ethanolamine, AMP (2-amino-2-methyl-1-propanol), piperazine, CAPS, 1,3-diaminopropane, CABS, and piperadine.

In some embodiments of any of the preceding methods, the thermostable nucleic acid polymerase is a thermostable DNA polymerase. Any suitable thermostable DNA polymerase may be used in the methods of the invention, for example, commercially available thermostable DNA polymerases, or any thermostable DNA polymerase described herein and/or known in the art. In some embodiments, the thermostable DNA polymerase is a wild-type thermostable DNA polymerase, e.g., Thermus aquaticus (Taq) DNA polymerase (see, e.g., U.S. Pat. No. 4,889,818), Thermus thermophilus (Tth) DNA polymerase (see, e.g., U.S. Pat. Nos. 5,192,674; 5,242,818; and 5,413,926), Thermus filiformis (Tfi) DNA polymerase, Thermus flavus (Tfl) DNA polymerase, Thermococcus litoralis (Tli) DNA polymerase (see, e.g., U.S. Pat. No. 5,332,785), Thermatoga maritima (Tma) DNA polymerase, Thermus spp. Z05 DNA polymerase, Tsp sps17 DNA polymerase derived from Thermus species spsl 7, now called Thermus oshimai (see, e.g., U.S. Pat. No. 5,405,774), Bacillus stearothermophilus (Bst) DNA polymerase (see, e.g., U.S. Pat. No. 5,747,298), an archaeal polymerase (e.g., thermostable DNA polymerases from hyperthermophylic archaeons Pyrococcus furiosus (e.g., Pfu; see, e.g., U.S. Pat. No. 5,948,663), KOD DNA polymerase derived from Pyrococcus sp. KOD1 (e.g., U.S. Pat. No. 6,033,859), Thermococcus litoralis (e.g., VENTR® (NEB)), and 9° NTM (NEB)), or a mutant, derivative, or fragment thereof having DNA polymerase activity (e.g., mutant DNA polymerases that include point mutations compared to a reference thermostable DNA polymerase sequence, e.g., Taq A271 F667Y, Tth A273 F668Y, and Taq A271 F667Y E681W; truncation mutants, e.g., KlenTAQ®, an N-terminal deletion variant of Taq lacking the first 280 amino acids; and mutants that include truncations and point mutations, e.g., Hemo KlenTaq®, an N-terminal deletion variant of Taq lacking the first 280 amino acids containing three internal point mutations that make it resistant to inhibitors in whole blood). For example, suitable DNA polymerases include, but are not limited to, Taq, Hemo KlenTaq®, Hawk Z05, APTATAQ™, Pfu, and VENTR®.

In some embodiments, the thermostable DNA polymerase is a mutant thermostable DNA polymerase. In some embodiments, the mutant thermostable DNA polymerase is listed in Table 2. In some embodiments, the mutant thermostable DNA polymerase is selected from the group consisting of Klentaq® 1, Klentaq® LA, Cesium Klentaq® AC, Cesium Klentaq® AC LA, Cesium Klentaq® C, Cesium Klentaq® C LA, Omni Klentaq®, Omni Klentaq® 2, Omni Klentaq® LA, Omni Taq, OmniTaq LA, Omni Taq 2, Omni Taq 3, Hemo KlenTaq®, KAPA Blood DNA polymerase, KAPA3G Plant DNA polymerase, KAPA 3G Robust DNA polymerase, MyTaq™ Blood, and PHUSION® Blood II DNA polymerase. In some embodiments, the thermostable DNA polymerase is a hot start thermostable DNA polymerase (e.g., APTATAQ™, Hawk Z05, or PHUSION® Blood II DNA polymerase).

In some embodiments, the thermostable nucleic acid polymerase (e.g., thermostable DNA polymerase) is inhibited by the presence of subject-derived cells or cell debris under normal reaction conditions. In some embodiments, the thermostable nucleic acid polymerase (e.g., thermostable DNA polymerase) is inhibited by whole blood under normal reaction conditions. In some embodiments, the thermostable nucleic acid polymerase (e.g., thermostable DNA polymerase) is inhibited by 1% (v/v) whole blood under normal reaction conditions. In some embodiments, the thermostable nucleic acid polymerase (e.g., thermostable DNA polymerase) is inhibited by 8% (v/v) whole blood under normal reaction conditions. In some embodiments, the normal reaction conditions are the reaction conditions recommended by the manufacturer of the thermostable DNA polymerase or reaction conditions that are commonly used in the art.

In some embodiments of any of the preceding methods, the method further includes amplifying and detecting one or more additional target nucleic acids in a multiplexed PCR reaction to generate one or more additional amplicons. In some embodiments, the multiplexed PCR reaction amplifies 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, or more target nucleic acids.

In some embodiments of any of the preceding methods, an amplicon is produced in the presence of at least 1 μg of subject DNA, e.g., at least 1 μg of subject DNA, at least 5 μg of subject DNA, at least 10 μg of subject DNA, at least 15 μg of subject DNA, at least 20 μg of subject DNA, at least 25 μg of subject DNA, at least 30 μg of subject DNA, at least 35 μg of subject DNA, at least 40 μg of subject DNA, at least 45 μg of subject DNA, at least 50 μg of subject DNA, at least 55 μg of subject DNA, or at least 60 μg of subject DNA.

In some embodiments of any of the preceding methods, the method results in the production of at least 105 copies of the amplicon, e.g., at least 105 copies, at least 106 copies, at least 107 copies, at least 108 copies, at least 109 copies, at least 1010 copies, at least 1011 copies, at least 1012 copies, at least 1013 copies, or at least 1014 copies of the amplicon. For example, in some embodiments, the method results in the production of at least 108 copies of the amplicon. In some embodiments, the method results in the production of at least 109 copies of the amplicon.

### Contamination Control

One potential problem in the use of amplification methods such as PCR as an analytical tool is the risk of having new reactions contaminated with old, amplified products. Potential sources of contamination include a) large numbers of target organisms in clinical specimens that may result in cross-contamination, b) plasmid clones derived from organisms that have been previously analyzed and that may be present in larger numbers in the laboratory environment, and c) repeated amplification of the same target sequence leading to accumulation of amplification products in the laboratory environment. A common source of the accumulation of the PCR amplicon is aerosolization of the product. Typically, if uncontrolled aerosolization occurs, the amplicon will contaminate laboratory reagents, equipment, and ventilation systems. When this happens, all reactions will be positive, and it is not possible to distinguish between amplified products from the contamination or a true, positive sample. In addition to taking precautions to avoid or control this carry-over of old products, preferred embodiments include a blank reference reaction in every PCR experiment to check for carry-over. For example, carry-over contamination will be visible on the agarose gel as faint bands or fluorescent signal when TaqMan® probes, molecular beacons, or intercalating dyes, among others, are employed as detection mechanisms. Furthermore, it is preferred to include a positive sample. As an example, in some embodiments, contamination control is performed using any of the approaches and methods described in WO 2012/054639. In some embodiments, a bleach solution is used to neutralize potential amplicons. In some embodiments, contamination control includes the use of ethylene oxide (EtO) treatment, for example, of cartridge components.

Typically, the instrumentation and processing areas for samples that undergo amplification are split into pre- and post-amplification zones. This minimizes the chances of contamination of samples with amplicon prior to amplification.

### Panels

The methods of the invention can be configured to detect a predetermined panel of pathogens. In some embodiments, the panel may be configured to individually detect between 1 and 250 (e.g., 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, or 250) pathogens, including pathogens selected from the following: Acinetobacter spp. (e.g., Acinetobacter baumannii, Acinetobacter pittii, and Acinetobacter nosocomialis), Enterobacteriaceae spp., Enterococcus spp. (e.g., Enterococcus faecium (including E. faecium with resistance marker vanA/B) and Enterococcus faecalis), Klebsiella spp. (e.g., Klebsiella pneumoniae (including, e.g., K. pneumoniae with resistance marker KPC), Klebsiella aerogenes, and Klebsiella oxytoca), Pseudomonas spp. (e.g., Pseudomonas aeruginosa), Staphylococcus spp. (including, e.g., Staphylococcus aureus (e.g., S. aureus with resistance marker mecA), Staphylococcus haemolyticus, Staphylococcus lugdunensis, Staphylococcus maltophilia, Staphylococcus saprophyticus, coagulase-positive Staphylococcus species, and coagulase-negative (CoNS) Staphylococcus species), Streptococcus spp. (e.g., Streptococcus mitis, Streptococcus pneumoniae, Streptococcus agalactiae, Streptococcus anginosa, Streptococcus bovis, Streptococcus dysgalactiae, Streptococcus mutans, Streptococcus sanguinis, and Streptococcus pyogenes), Escherichia spp. (e.g., Escherichia coli), Stenotrophomonas spp. (e.g., Stenotrophomonas maltophilia), Proteus spp. (e.g., Proteus mirabilis and Proteus vulgaris), Serratia spp. (e.g., Serratia marcescens), Citrobacter spp. (e.g., Citrobacter freundii and Citrobacter koseri), Haemophilus spp. (e.g., Haemophilus influenzae), Listeria spp. (e.g., Listeria monocytogenes), Neisseria spp. (e.g., Neisseria meningitidis), Bacteroides spp. (e.g., Bacteroides fragilis), Burkholderia spp. (e.g., Burkholderia cepacia), Campylobacter (e.g., Campylobacter jejuni and Campylobacter coli), Clostridium spp. (e.g., Clostridium perfringens), Kingella spp. (e.g., Kingella kingae), Morganella spp. (e.g., Morganella morgana), Prevotella spp. (e.g., Prevotella buccae, Prevotella intermedia, and Prevotella melaninogenica), Propionibacterium spp. (e.g., Propionibacterium acnes), Salmonella spp. (e.g., Salmonella enterica), Shigella spp. (e.g., Shigella dysenteriae and Shigella flexneri), and Enterobacter spp. (e.g., Enterobacter aerogenes and Enterobacter cloacae), Borrelia spp., (e.g., Borrelia burgdorferi sensu lato (Borrelia burgdorferi, Borrelia afzelii, and Borrelia garinii) species), Rickettsia spp. (including Rickettsia rickettsii and Rickettsia parkeri), Ehrlichia spp. (including Ehrlichia chaffeensis, Ehrlichia ewingii, and Ehrlichia muris-like), Coxiella spp. (including Coxiella burnetii), Anaplasma spp. (including Anaplasma phagocytophilum), Francisella spp., (including Francisella tularensis (including Francisella tularensis subspp. holarctica, mediasiatica, and novicida)), Streptococcus spp. (including Streptococcus pneumonia), and Neisseria spp. (including Neisseria meningitidis). In some embodiments, the bacterial pathogen panel is further configured to detect a fungal pathogen, for example, Candida spp. (e.g., Candida albicans, Candida guilliermondii, Candida glabrata, Candida krusei, Candida lusitaniae, Candida parapsilosis, Candida dubliniensis, Candida kefyr, and Candida tropicalis) and Aspergillus spp. (e.g., Aspergillus fumigatus). In some embodiments, the pathogen panel is further configured to detect a Candida spp. (including Candida albicans, Candida guilliermondii, Candida glabrata, Candida krusei, Candida lusitaniae, Candida parapsilosis, Candida dubliniensis, Candida kefyr, and Candida tropicalis). In cases where multiple species of a genus are detected, the species may be detected using individual target nucleic acids or using target nucleic acids that are universal to all of the species, for example, target nucleic acids amplified using universal primers. In some embodiments, the panel may be configured to detect 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, or 100 or more pathogens. In some embodiments, the panel may be configured to detect 40-50 pathogens, 42-50 pathogens, 44-50 pathogens, 46-50 pathogens, 48-50 pathogens, 44-50 pathogens, 46-50 pathogens, 48-50 pathogens, 40-48 pathogens, 42-48 pathogens, 44-48 pathogens, 46-48 pathogens, 48-48 pathogens, 44-48 pathogens, 46-48 pathogens, 40-46 pathogens, 42-46 pathogens, 44-46 pathogens, 40-44 pathogens, 42-44 pathogens, or 40-42 pathogens. In some embodiments, the panel may be configured to detect 48 pathogens.

In some embodiments, the panel may be configured to individually detect one or more (e.g., 1, 2, 3, 4, 5, 6, or 7) of Acinetobacter baumannii, Enterococcus faecium, Enterococcus faecalis, Klebsiella pneumoniae, Pseudomonas aeruginosa, Escherichia coli, and Staphylococcus aureus.

In some embodiments, the panel may be configured to individually detect one or more (e.g., 1, 2, 3, 4, 5, 6, 7, 8, or 9) Candida spp. (e.g., Candida albicans, Candida guilliermondii, Candida glabrata, Candida krusei, Candida lusitaniae, Candida parapsilosis, Candida dubliniensis, Candida kefyr, and Candida tropicalis).

In some embodiments, the panel can be a Lyme disease pathogen panel configured to individually detect one, two, or three Borrelia burgdorferi sensu lato (Borrelia burgdorferi, Borrelia afzelii, and Borrelia garinii) species. These species may be detected using individual target nucleic acids or using target nucleic acids that are universal to all three species, for example, target nucleic acids amplified using universal primers. In some embodiments, the panel is configured to detect Borrelia burgdorferi. In some embodiments, the panel is configured to detect Borrelia afzelii. In some embodiments, the panel is configured to detect Borrelia garinii. In some embodiments, the panel is configured to detect Borrelia burgdorferi and Borrelia afzelii. In some embodiments, the panel is configured to detect Borrelia burgdorferi and Borrelia garinii. In some embodiments, the panel is configured to detect Borrelia afzelii and Borrelia garinii. In some embodiments, the panel is configured to detect Borrelia burgdorferi, Borrelia afzelii and Borrelia garinii. In some embodiments, the panel may be configured to individually detect one or more (e.g., 1, 2, 3, 4, 5, or 6) of Rickettsia rickettsii, Coxiella burnettii, Ehrlichia chaffeensis, Babesia microti, Francisella tularensis, and Anaplasma phagocytophilum.

In any of the above embodiments, the panel may be configured to detect a marker that is characteristic of a genus, for example, a pan-bacterial marker, a pan Gram positive marker, a pan Gram negative marker, a pan-Candida marker, or a pan-Borrelia marker. In any of the above panels, the analyte may be a nucleic acid (e.g., an amplified target nucleic acid, as described above), or a polypeptide (e.g., a polypeptide derived from the pathogen or a pathogen-specific antibody produced by a host subject, for example, an IgM or IgG antibody). In some embodiments, multiple analytes (e.g., multiple amplicons) are used to detect a pathogen. In any of the above panels, the biological sample may be a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), or sputum. In some embodiments, the biological sample is blood (e.g., whole blood, a crude whole blood lysate, serum, or plasma). Such panels may be used, for example, to diagnose bloodstream infections. In some embodiments, the biological sample may be a tissue sample, for example, a homogenized tissue sample. Such panels may be used, for example, to detect infections present in tissue, e.g., tissue biopsies of skin at the site of a tick bite to identify Borrelia spp. for diagnosis of Lyme disease.

For example, provided herein is a panel including at least 20 pathogen target nucleic acids (e.g., at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pathogen target nucleic acids), wherein the panel includes (i) one or more genus-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more genus-level target nucleic acids), (ii) one or more Gram positive bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram positive bacterial target nucleic acids), (iii) one or more Gram negative bacterial target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more Gram negative bacterial target nucleic acids), and/or (iv) one or more resistance gene target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more resistance gene target nucleic acids). In some embodiments, the panel includes at least 28 pathogen target nucleic acids.

In any of the panels described herein, the panel may further include (v) one or more pan-level target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more pan-level target nucleic acids).

In any of the panels described herein, the panel may further include (vi) one or more fungal target nucleic acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more fungal target nucleic acids).

Any of the panels described herein may have a percent coverage of greater than or equal to 90% (e.g., 90%, 91%, 92%, 93%, 94%, 95%, 96%, 97%, 98%, 99%, or 100%) of pathogen species associated with infections of the sample. For example, in some embodiments, the panel has a percent coverage of greater than or equal to 95%, 96%, 97%, 98%, or 99% of pathogen species associated with infections of the sample. In some embodiments, the panel has a percent coverage of greater than or equal to 99% of pathogen species associated with infections of the sample.

The panels described herein may be split across one or more subpanels. Any suitable number of subpanels may be used in any of the methods described herein. For example, in some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty subpanels. In some embodiments, the panel includes at least two subpanels. In some embodiments, the panel includes at least four subpanels. In some embodiments, the panel includes four subpanels. In some embodiments, the panel includes five subpanels.

Each subpanel may include any suitable number of pathogen target nucleic acids. For example, in some embodiments, the subpanel may include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more pathogen target nucleic acids. For example, in some embodiments, the subpanel includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, or more pathogen target nucleic acids. In some embodiments, each subpanel includes at least 6 pathogen target nucleic acids. In some embodiments, each subpanel includes 9 pathogen target nucleic acids. In some embodiments, each subpanel includes 14 pathogen target nucleic acids. In some embodiments, each subpanel includes an internal control channel.

The panels described herein may include any suitable number of pathogen target nucleic acids. For example, in some embodiments, the panel includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 84, 86, 87, 88, 89, 90, 91, 92, 93, 94, 95, 96, 97, 98, 99, 100, or more target nucleic acids. In some embodiments, the panel includes at least 28 pathogen target nucleic acids. In some embodiments, the panel includes at least 30 pathogen target nucleic acids. In some embodiments, the panel includes at least 32 pathogen target nucleic acids. In some embodiments, the panel includes at least 36 pathogen target nucleic acids. In some embodiments, the panel includes at least 36 pathogen target nucleic acids. In some embodiments, the panel includes at least 38 pathogen target nucleic acids. In some embodiments, the panel includes at least 40 pathogen target nucleic acids. In some embodiments, the panel includes at least 42 pathogen target nucleic acids. In some embodiments, the panel includes at least 44 pathogen target nucleic acids. In some embodiments, the panel includes 45 pathogen target nucleic acids. In some embodiments, the 45 pathogen target nucleic acids are split between five subpanels. In some embodiments, the panel includes at least 48 pathogen target nucleic acids. In some embodiments, the 48 pathogen target nucleic acids are split between four subpanels.

For example, in some embodiments, the panel includes between 15 and 80, between 15 and 75, between 15 and 70, between 15 and 65, between 15 and 60, between 15 and 55, between 15 and 50, between 15 and 45, between 15 and 40, between 15 and 35, between 15 and 30, between 15 and 25, between 15 and 20, between 20 and 80, between 20 and 75, between 20 and 70, between 20 and 65, between 20 and 60, between 20 and 55, between 20 and 50, between 20 and 45, between 20 and 40, between 20 and 35, between 20 and 30, between 20 and 25, between 25 and 80, between 25 and 75, between 25 and 70, between 25 and 65, between 25 and 60, between 25 and 55, between 25 and 50, between 25 and 45, between 25 and 40, between 25 and 35, between 25 and 30, between 30 and 80, between 30 and 75, between 30 and 70, between 30 and 65, between 30 and 60, between 30 and 55, between 30 and 50, between 30 and 45, between 30 and 40, between 30 and 35, between 35 and 80, between 35 and 75, between 35 and 70, between 35 and 65, between 35 and 60, between 35 and 55, between 35 and 50, between 35 and 45, between 35 and 40, between 40 and 80, between 40 and 75, between 40 and 70, between 40 and 65, between 40 and 60, between 40 and 55, between 40 and 50, between 40 and 45, between 45 and 80, between 45 and 75, between 45 and 70, between 45 and 65, between 45 and 60, between 45 and 55, between 45 and 50, between 50 and 80, between 50 and 75, between 50 and 70, between 50 and 65, between 50 and 60, between 50 and 55, between 55 and 80, between 55 and 75, between 55 and 70, between 55 and 65, between 55 and 60, between 60 and 80, between 60 and 75, between 60 and 70, between 60 and 65, between 65 and 80, between 65 and 75, between 65 and 70, between 65 and 65, between 70 and 80, between 70 and 75, or between 75 and 80 pathogen target nucleic acids.

Any suitable genus-level target nucleic acids may be included in any of the panels described herein. For example, in some embodiments, the one or more genus-level target nucleic acids are characteristic of a genus selected from the group consisting of Acinetobacter spp., anaerobes, Citrobacter spp., Clostridium spp., Corynebacterium spp., Enterobacter spp., Acinetobacter baumannii-calcoaceticus complex, Enterobacterales, Enterobacter cloacae complex, Enterobacteriaceae, Enterococcus spp., Klebsiella spp., Mycobacterium spp., Neisseria spp., Salmonella spp., Staphylococcus spp., coagulase negative Staphylococcus spp. (CoNS), Streptococcus spp., Viridans group Streptococcus, Aspergillus spp., Candida spp., Proteus spp., Serratia spp., Clostridium spp., Bacteroides spp., and Cryptococcus spp. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty genus-level target nucleic acids selected from the group consisting of Acinetobacter spp., anaerobes, Citrobacter spp., Clostridium spp., Corynebacterium spp., Enterobacter spp., Enterobacter cloacae complex, Enterobacteriaceae, Enterococcus spp., Klebsiella spp., Mycobacterium spp., Neisseria spp., Salmonella spp., Staphylococcus spp., coagulase negative Staphylococcus spp., Streptococcus spp., Viridans group Streptococcus, Aspergillus spp., Candida spp., and Cryptococcus spp. In some embodiments, the genus-level target nucleic acid characteristic of Enterobacteriaceae is characteristic of Klebsiella spp., Enterobacter spp., Citrobacter spp., Serratia spp., Proteus spp., and/or Morganella spp. In some embodiments, the genus-level target nucleic acid characteristic of coagulase negative Staphylococcus spp. is characteristic of S. epidermidis, S. haemolyticus, S. lugdunensis, and/or S. hominis. In some embodiments, the genus-level target nucleic acid characteristic of Viridans group Streptococcus is characteristic of S. anginosus, S. mitis, and/or S. oralis. In some embodiments, the genus-level target nucleic acid characteristic of anaerobes is characteristic of Clostridium spp. and/or Bacteroides spp.

Any suitable Gram positive bacterial target nucleic acid may be included in any of the panels described herein. In some embodiments, the one or more Gram positive bacterial target nucleic acids are selected from the group consisting of E. faecium, E. faecalis, S. aureus, S. pneumoniae, S. pyogenes, and S. agalactiae. In some embodiments, the panel includes at least two, at least three, at least four, at least five, or all six Gram positive bacterial target nucleic acids selected from the group consisting of E. faecium, E. faecalis, S. aureus, S. pneumoniae, S. pyogenes, and S. agalactiae. In some embodiments, the one or more Gram positive bacterial target nucleic acids is amplified in the presence of a forward primer and a reverse primer set forth in Table 3 of U.S. Provisional Patent Application No. 62/863,538 or PCT Publication No. WO 2020/257691, each of which is incorporated by reference herein in its entirety. In some embodiments, the one or more Gram positive bacterial target nucleic acid amplicons is detected using a 5′ capture probe and a 3′ capture probe set forth in Table 4 of U.S. Provisional Patent Application No. 62/863,538 or PCT Publication No. WO 2020/257691.

Any suitable Gram negative bacterial target nucleic acid may included in any of the panels described herein. In some embodiments, the one or more Gram negative bacterial target nucleic acids are selected from the group consisting of A. baumannii, E. coli, H. influenzae, Klebsiella pneumoniae, Klebsiella oxytoca, Klebsiella variicola, P. aeruginosa, S. marcescens, P. mirabilis, and S. maltophilia. In some embodiments, the panel includes at least two, at least three, at least four, at least five, at least six, at least seven, at least eight, at least nine, or all ten Gram negative bacterial target nucleic acids selected from the group consisting of A. baumannii, E. coli, H. influenzae, Klebsiella pneumoniae, Klebsiella oxytoca, Klebsiella variicola, P. aeruginosa, S. marcescens, P. mirabilis, and S. maltophilia. In some embodiments, the one or more Gram negative bacterial target nucleic acids is amplified in the presence of a forward primer and a reverse primer set forth in Table 3 of U.S. Provisional Patent Application No. 62/863,538 or PCT Publication No. WO 2020/257691. In some embodiments, the one or more Gram negative bacterial target nucleic acid amplicons is detected using a 5′ capture probe and a 3′ capture probe set forth in Table 4 of U.S. Provisional Patent Application No. 62/863,538 or PCT Publication No. WO 2020/257691.

Any suitable resistance gene target nucleic acid may be included in any of the panels described herein. In some embodiments, the one or more resistance gene target nucleic acids are selected from the group consisting of mecA, mecC, mefA, mefE, MCR (e.g., mcr-1), vanA, vanB, ermA, ermB, KPC, NDM, VIM, IMP, OXA-23-like, OXA-48-like, SHV, CMY, DHA, CTX-M (e.g., CTX-M 14 and/or CTX-M 15), TEM, FKS, PDR1, and ERG11. In some embodiments, the panel includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, or all twenty-four resistance gene target nucleic acids selected from the group consisting of mecA, mecC, mefA, mefE, MCR (e.g., mcr-1), vanA, vanB, ermA, ermB, KPC, NDM, VIM, IMP, OXA-23-like, OXA-48-like, SHV, CMY, DHA, CTX-M (e.g., CTX-M 14 and/or CTX-M 15), TEM, FKS, PDR1, and ERG11. In some embodiments, the resistance gene target nucleic acid is characteristic of mecA and mecC; mefA and mefE; vanA and vanB; ermA and ermB; NDM, VIM, and IMP; CMY and DHA; or CTX-M (e.g., CTX-M 14 and CTX M 15). In some embodiments, the one or more resistance gene target nucleic acids is amplified in the presence of a forward primer and a reverse set forth in Table 10, Table 12, Table 14, or Table 16 of U.S. Provisional Patent Application No. 62/863,538 or PCT Publication No. WO 2020/257691. In some embodiments, the one or more resistance gene target nucleic acid amplicons is detected using a 5′ capture probe and a 3′ capture probe set forth in Table 11, Table 13, Table 15, or Table 17 of U.S. Provisional Patent Application No. 62/863,538 or PCT Publication No. WO 2020/257691.

In some examples, any of the panels described herein may include any of the members of panels described in PCT Publication No. WO 2020/072858 and U.S. patent application Ser. No. 17/282,305, which are incorporated by reference herein in their entirety. For example, in some embodiments, the panel may include any of those described in Tables 1-15 or 19 of WO 2020/072858 and U.S. patent application Ser. No. 17/282,305. In some embodiments, the one or more resistance gene target nucleic acids is amplified in the presence of a forward primer and a reverse set forth in Tables 16, 17, or 20 or Example 6 of WO 2020/072858 and U.S. patent application Ser. No. 17/282,305. In some embodiments, the one or more resistance gene target nucleic acid amplicons is detected using a 5′ capture probe and a 3′ capture probe set forth in Tables 16, 17, or 20 or Example 6 of WO 2020/072858 and U.S. patent application Ser. No. 17/282,305.

Any suitable pan-level target nucleic acid may be included in any of the panels described herein. In some embodiments, the one or more pan-level target nucleic acids are selected from the group consisting of Pan-Bacterial, Pan-Gram positive, Pan-Gram negative, and Pan-Fungal. In some embodiments, the panel includes at least two, at least three, or all four pan-level target nucleic acids selected from the group consisting of Pan-Bacterial, Pan-Gram positive, Pan-Gram negative, and Pan-Fungal.

Any suitable fungal target nucleic acid may be included in any of the panels described herein. In some embodiments, the one or more fungal target nucleic acids are selected from the group consisting of C. albicans, C. tropicalis, C. dubliniensis, C. parapsilosis, C. krusei, C. glabrata, C. auris, C. lusitaniae, C. haemulonii, C. duobushaemulonii, C. kefyr, and C. pseudohaemulonii. In some embodiments, the panel includes at least two, at least three, at least four, at least five, at least six, at least seven, at least eight, at least nine, at least ten, at least eleven, or all twelve fungal target nucleic acids selected from the group consisting of C. albicans, C. tropicalis, C. dubliniensis, C. parapsilosis, C. krusei, C. glabrata, C. auris, C. lusitaniae, C. haemulonii, C. duobushaemulonii, C. kefyr, and C. pseudohaemulonii. In some embodiments, the one or more fungal target nucleic acids is amplified in the presence of a forward primer and a reverse primer set forth in Table 7 of U.S. Provisional Patent Application No. 62/863,538 or PCT Publication No. WO 2020/257691. In some embodiments, the one or more fungal target nucleic acid amplicons is detected using a 5′ capture probe and a 3′ capture probe set forth in Table 8 or Table 9 of U.S. Provisional Patent Application No. 62/863,538 or PCT Publication No. WO 2020/257691. In some embodiments, the panel includes at least two, at least three, at least four, at least five, at least six, at least seven, at least eight, or all nine fungal target nucleic acids selected from the group consisting of Candida spp., C. albicans, C. tropicalis, C. parapsilosis, C. krusei, C. glabrata, C. auris, Aspergillus spp., and Cryptococcus spp.

In some embodiments, the panel is any panel or set of targets described in U.S. Provisional Patent Application No. 62/863,538 or PCT Publication No. WO 2020/257691. In some embodiments, the panel is a panel shown in any one of Tables 20-24 of U.S. Provisional Patent Application No. 62/863,538 or PCT Publication No. WO 2020/257691.

In some embodiments, the panel includes: (i) a first subpanel including the following pathogen target nucleic acids: Pan Gram negative, E. coli, K. pneumoniae, Enterobacter spp., Enterobacter cloacae complex, Citrobacter spp., S. marcescens, P. mirabilis, Salmonella spp., and an internal control; (ii) a second subpanel including the following pathogen target nucleic acids: Acinetobacter spp., A. baumanii, P. aeruginosa, S. maltophilia, H. influenzae, KPC, NDM/VIM/IMP, OXA-48-like, CTX-M (e.g., CTX-M 14/15), and an internal control; (iii) a third subpanel including the following pathogen target nucleic acids: Pan Gram positive, Enterococcus spp., E. faecium, E. faecalis, Staphylococcus spp., S. aureus, coagulase negative Staphylococcus spp., mecA/C, vanA/B, and an internal control; (iv) a fourth subpanel including the following pathogen target nucleic acids: Streptococcus spp., S. pneumoniae, S. pyogenes, S. agalactiae, Viridans Group Streptococcus, Anaerobes, Corynebacterium spp., ermA/B, mefA/E, and an internal control; and (v) a fifth subpanel including the following pathogen target nucleic acids: Candida spp., C. albicans, C. tropicalis, C. parapsilosis, C. krusei, C. glabrata, C. auris, Aspergillus spp., Cryptococcus spp., and an internal control.

For example, in some embodiments, the panel includes one or more targets from Table 3 below. In some embodiments, the panel comprises or consists of the panel shown in Table 3. In some embodiments, the panel includes (i) a first subpanel comprising one or more of the following target nucleic acids: Staphylococcus aureus, Coagulase negative staphylococci (CoNS), mecA, mecC, Streptococcus agalactiae, Escherichia coli, Klebsiella pneumoniae, Klebsiella oxytoca, Klebsiella aerogenes, Enterobacter cloacae complex, Pseudomonas aeruginosa, and Acinetobacter baumannii-calcoaceticus complex; (ii) a second subpanel comprising one or more of the following target nucleic acids: Enterobacterales, KPC, NDM, VIM, IMP, OXA-48-like, CTX-M (e.g., CTX-M 14/15), CMY, DHA, FOX, mcr-1, and OXA-23-like; (iii) a third subpanel comprising one or more of the following target nucleic acids: Enterococcus faecalis, Enterococcus faecium, vanA/B, Streptococcus spp., Streptococcus pneumoniae, Streptococcus pyogenes, Staphylococcus lugdunensis, Citrobacter spp., Proteus spp., Serratia spp., Clostridium spp., and Bacteroides spp.; and/or (iv) a fourth subpanel comprising one or more of the following target nucleic acids: Candida albicans, Candida tropicalis, Candida glabrata, Candida krusei, Candida parapsilosis, Candida auris, Candida lusitaniae, Candida dubliniensis, Candida kefyr, Stenotrophomonas maltophilia, Pan Gram negative, and Pan Gram positive. In some embodiments, the first subpanel further includes an internal control. In some embodiments, the second subpanel further includes an internal control. In some embodiments, the third subpanel further includes an internal control. In some embodiments, the fourth subpanel further includes an internal control. In some embodiments, the first, second, third, and fourth subpanels each further include an internal control.

TABLE 3
Exemplary Panel

| Multiplex 1 | Multiplex 2 | Multiplex 3 | Multiplex 4 |
|---|---|---|---|
| Staphylococcus | Enterobacterales | Enterococcus | Candida albicans |
| aureus | | faecalis | |
| Coagulase | KPC | Enterococcus | Candida |
| negative | | faecium | tropicalis |
| staphylococci | | | |
| (CoNS) | | | |
| mecA | NDM | vanA/B | Candida glabrata |
| mecC | VIM | Streptococcus | Candida krusei |
| | | spp. | |
| Streptococcus | IMP | Streptococcus | Candida |
| agalactiae | | pneumoniae | parapsilosis |
| Escherichia | OXA-48-like | Streptococcus | Candida auris |
| coli | | pyogenes | |
| Klebsiella | CTX-M | Staphylococcus | Candida |
| pneumoniae | | lugdunensis | lusitaniae |
| Klebsiella | CMY | Citrobacter | Candida |
| oxytoca | | spp. | dubliniensis |
| Klebsiella | DHA | Proteus spp. | Candida kefyr |
| aerogenes | | | |
| Enterobacter | FOX | Serratia spp. | Stenotrophomonas |
| cloacae | | | maltophilia |
| complex | | | |
| Pseudomonas | mcr-1 | Clostridium | Pan Gram negative |
| aeruginosa | | spp. | |
| Acinetobacter | OXA-23-like | Bacteroides | Pan Gram positive |
| baumannii- | | spp. | |
| calcoaceticus | | | |
| complex | | | |
| IC | IC | IC | IC |

In other embodiments, the panel may include (i) a first subpanel comprising one or more (e.g., 1, 2, 3, 4, 5, 6, 7, 8, 9, 10, or all 11) of the following target nucleic acids: Staphylococcus aureus, Coagulase negative staphylococci (CoNS), mecA, Streptococcus agalactiae, Escherichia coli, Klebsiella pneumoniae, Klebsiella oxytoca, Klebsiella aerogenes, Enterobacter cloacae complex, Pseudomonas aeruginosa, and Acinetobacter baumannii-calcoaceticus complex; (ii) a second subpanel comprising one or more (e.g., 1, 2, 3, 4, 5, 6, 7, 8, 9, or all 10) of the following target nucleic acids: Enterobacterales, KPC, NDM, VIM, IMP, OXA-48-like, CTX-M (e.g., CTX-M 14/15), CMY, DHA, and OXA-23-like; (iii) a third subpanel comprising one or more (e.g., 1, 2, 3, 4, 5, 6, 7, 8, 9, 10, 11, or all 12) of the following target nucleic acids: Enterococcus faecalis, Enterococcus faecium, vanA/B, Streptococcus spp., Streptococcus pneumoniae, Streptococcus pyogenes, Staphylococcus lugdunensis, Citrobacter spp., Proteus spp., Serratia spp., Clostridium spp., and Bacteroides spp.; and/or (iv) a fourth subpanel comprising one or more (e.g., 1, 2, 3, 4, 5, 6, 7, 8, or all 9) of the following target nucleic acids: Candida albicans, Candida tropicalis, Candida glabrata, Candida krusei, Candida parapsilosis, Candida auris, Candida lusitaniae, Candida dubliniensis, and Stenotrophomonas maltophilia.

The group of Enterobacter cloacae complex is described, e.g., in Mezzatesta et al. Future Microbiology 7:887-902, 2012, which is incorporated by reference herein in its entirety. This group may include, e.g., Enterobacter cloacae, Enterobacter asburiae, Enterobacter hormaechei, Enterobacter kobei, Enterobacter ludwigii, and/or Enterobacter nimipressuralis. A target nucleic acid characteristic of Enterobacter cloacae complex may be characteristic of one or more (e.g., 1, 2, 3, 4, 5, or all 6) of Enterobacter cloacae, Enterobacter asburiae, Enterobacter hormaechei, Enterobacter kobei, Enterobacter ludwigii, and/or Enterobacter nimipressuralis.

The group of Acinetobacter baumannii-calcoaceticus complex is described, e.g., in Nemec et al. Res. Microbiol. 162 (4): 393-404, 2011, which is incorporated by reference herein in its entirety. This group may include, e.g., Acinetobacter calcoaceticus, Acinetobacter baumannii, Acinetobacter pittii and/or Acinetobacter nosocomialis. A target nucleic acid characteristic of Acinetobacter baumannii-calcoaceticus complex may be characteristic of one or more (e.g., 1, 2, 3, or all 4) of Acinetobacter calcoaceticus, Acinetobacter baumannii, Acinetobacter pittii and/or Acinetobacter nosocomialis.

Any of the preceding panels may be further configured to configured to individually detect one or more (e.g., 1, 2, 3, 4, 5, 6, or 7) of Acinetobacter baumannii, Enterococcus faecium, Enterococcus faecalis, Klebsiella pneumoniae, Pseudomonas aeruginosa, Escherichia coli, and Staphylococcus aureus. For example, in some embodiments, the panel may be configured to individually detect one or more (e.g., 1, 2, 3, 4, or 5) of Enterococcus faecium, Klebsiella pneumoniae, Pseudomonas aeruginosa, Escherichia coli, and Staphylococcus aureus, as in the FDA-cleared T2BACTERIA® panel (T2 Biosystems, Inc.).

Any of the preceding panels may be further configured to individually detect one or more (e.g., 1, 2, 3, 4, 5, 6, or 8) Candida spp. (e.g., Candida albicans, Candida guilliermondii, Candida glabrata, Candida krusei, Candida lusitaniae, Candida parapsilosis, Candida dubliniensis, and Candida tropicalis). For example, in some embodiments, the panel may be configured to individually detect one or more (e.g., 1, 2, 3, 4, or 5) of Candida albicans, Candida tropicalis, Candida krusei, Candida glabrata, and Candida parapsilosis, as in the FDA-cleared T2CANDIDA® panel (T2 Biosystems, Inc.).

Any of the preceding panels may be configured to individually detect one or more (e.g., 1, 2, 3, 4, 5, or 6) of Bacillus anthracis pX01 plasmid, Bacillus anthracis pX02 plasmid, Francisella tularensis, Burkholderia spp., Yersinia pestis, and Rickettsia prowazekii, or any other panel described in PCT Publication No. WO 2020/252084, which is incorporated by reference herein in its entirety.

Amplifying Multiple Amplicons Characteristic of a Species for Improved Sensitivity and/or Specificity

In some embodiments, the methods disclosed herein may involve amplification and detection of more than one amplicon characteristic of a species in a biological sample containing cells and/or cell debris including but not limited to blood (e.g., whole blood, a crude whole blood lysate, serum, or plasma), bloody fluids (e.g., wound exudate, phlegm, bile, and the like), tissue samples (e.g., tissue biopsies, including homogenized tissue samples), or sputum. In some embodiments, amplification of more than one target nucleic acid characteristic of a species increases the total amount of amplicons characteristic of the species in an assay (in other words, the amount of analyte is increased in the assay). This increase may allow, for example, an increase in sensitivity and/or specificity of detection of the species compared to a method that involves amplification and detection of a single amplicon characteristic of a species. In some embodiments, the methods of the invention may involve amplifying 2, 3, 4, 5, 6, 7, 8, 9, or 10 amplicons characteristic of a species.

In some embodiments, multiple (e.g., 2, 3, 4, 5, 6, 7, 8, 9, or 10) single-copy loci from a species are amplified and detected. In some embodiments, 2 single-copy loci from a species are amplified and detected. In some embodiments, amplification and detection of multiple single-copy loci from a species may allow for a sensitivity of detection comparable with methods that involve detecting an amplicon that is derived from a multi-copy locus. In some embodiments, methods involving detection of multiple single-copy loci amplified from a microbial species can detect from about 1-10 cells/mL (e.g., 1, 2, 3, 4, 5, 6, 7, 8, 9 or 10 cells/mL) of the microbial species in a liquid sample. In some embodiments, methods involving detection of multiple single-copy loci amplified from a microbial species have at least 95% correct detection when the microbial species is present in the liquid sample at a frequency of less than or equal to 5 cells/mL (e.g., 1, 2, 3, 4, or 5 cells/mL) of liquid sample.

The disclosure also provides embodiments in which at least three amplicons are produced by amplification of two target nucleic acids, each of which is characteristic of a species. For example, in some embodiments, a first target nucleic acid and a second target nucleic acid to be amplified may be separated (for example, on a chromosome or on a plasmid) by a distance ranging from about 50 base pairs to about 1000 1500 base pairs (bp), e.g., about 50, 100, 150, 200, 250, 300, 400, 500, 600, 700, 800, 900, or 1000, 1100, 1200, 1300, 1400, or 1500 bp base pairs. In some embodiments, a first target nucleic acid and a second target nucleic acid to be amplified may be separated (for example, on a chromosome or on a plasmid) by a distance ranging from about 50 bp to about 1000 bp (e.g., about 50, 100, 150, 200, 250, 300, 400, 500, 600, 700, 800, 900, or 1000 bp). In some embodiments the first target nucleic acid and the second target nucleic acid to be amplified may be separated by a distance ranging from about 50 bp to about 1500 bp, from about 50 bp to about 1400 bp, from about 50 bp to about 1300 bp, from about 50 bp to about 1200 bp, from about 50 bp to about 1100 bp, from about 50 bp to about 1000 bp, from about 50 bp to about 950 bp, from about 50 bp to about 900 bp, from about 50 bp to about 850 bp, from about 50 bp to about 800 bp, from about 50 bp to about 800 bp, from about 50 bp to about 750 bp, from about 50 bp to about 700 bp, from about 50 bp to about 650 bp, from about 50 bp to about 600 bp, from about 50 bp to about 550 bp, from about 50 bp to about 500 bp, from about 50 bp to about 500 bp, from about 50 bp to about 450 bp, from about 50 bp to about 400 bp, from about 50 bp to about 350 bp, from about 50 bp to about 300 bp, from about 50 bp to about 250 bp, from about 50 bp to about 200 bp, from about 50 bp to about 150 bp, or from about 50 bp to about 100 bp. In some embodiments, amplification of the first and second target nucleic acids using individual primer pairs (each having a forward and a reverse primer) may lead to amplification of an amplicon that includes the first target nucleic acid, an amplicon that includes the second target nucleic acid, and an amplicon that contains both the first and the second target nucleic acid. This may result in an increase in sensitivity of detection of the species compared to samples in which the third amplicon is not present. In any of the preceding embodiments, amplification may be by asymmetric PCR.

### EXAMPLES

The following examples are put forth so as to provide those of ordinary skill in the art with a complete disclosure and description of how the devices, systems, and methods described herein are performed, made, and evaluated, and are intended to be purely exemplary of the invention and are not intended to limit the scope of what the inventors regard as their invention.

### Example 1: Two-Stage PCR Methods

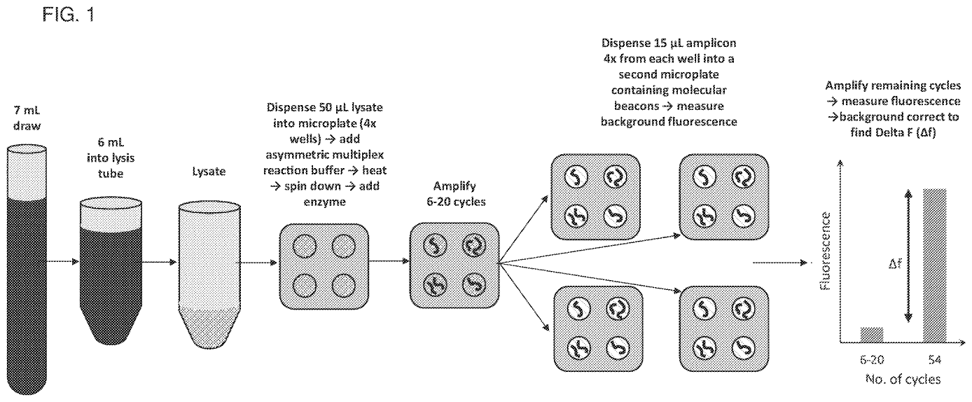

An exemplary two-stage asymmetric PCR method is outlined in FIG. 1. Whole blood samples were prepared with 20 colony forming units (CFU)/mL Enterococcus faecium, Serratia marcescens, and Streptococcus pneumoniae. 2 mL of blood was added into a lysis tube containing lysis buffer and zirconium oxide beads and centrifuged for 5 min at 6000×g. The supernatant was aspirated and 150 μL of TE buffer was added. The pellet was homogenized briefly on a vortexer and then centrifuged for 5 min at 6000×g. The supernatant was aspirated and 110 μL of TE was added, the tube was vortexed at 3200 rpm for 5 min, and then centrifuged for 2 min at 6000×g. In other examples, 6 mL of blood may be lysed as a single volume, centrifuged (e.g., as described above), and 400-2400 μL (e.g., 1200 μL) of TE buffer can be added, followed by brief homogenization, centrifugation, aspiration of the supernatant, and addition of buffer (e.g., TE buffer, e.g., 200-500 μL, e.g., 300 μL).

50 μL of lysate from the lysis tube was added to a 0.2 mL PCR tube, and 30 μL of reaction buffer containing an asymmetric multiplex primer mixture was added. The PCR tube was heated to 95° C. for 5 min, centrifuged for 5 min at 8000×g, and cooled to 25° C. 23 μL of formulated thermostable DNA polymerase enzyme was added.

The PCR tube was loaded into an EPPENDORF MASTERCYCLER® Pro and incubated for 5 min at 95° C. 8 amplification cycles were run in the EPPENDORF MASTERCYCLER® Pro, with hold times of 20 s denaturation, 30 s annealing, and 30 s extension at 95° C., 58° C., and 68° C., respectively, and then cooled to 4° C. In other examples, the first stage of amplification may occur in a multi-well plate (e.g., a 4-well plate).

The amplicon (supernatant) was transferred in 15 μL volumes into a 96-well plate containing a 5 μL mix of molecular beacons. 10 μL of mineral oil was added on top of each well. The entire plate was sealed with a plastic seal.

Fluorescence was measured on a Tecan INFINITE® 200 M monochromator plate reader, using the following settings: Green/FAM: 494 nm excitation, 527 to 537 nm emission; Yellow/HEX: 535 nm excitation, 567 to 577 nm emission; Red/Cy5: 647 nm excitation/679 to 689 nm emission. This measurement is the initial (8-cycle amplified) fluorescence measurement.

The plate was added into EPPENDORF MASTERCYCLER® Pro S and cycled for 46 additional cycles with the same PCR settings as indicated above to generate an asymmetric amplicon.

The plate was cooled down to 4° C.

Fluorescence was measured again using the same settings as after the initial amplification. This is the final fluorescence measurement.

The response (“Delta F”) is the final fluorescence at 54 cycles minus the fluorescence at 8 cycles within each well.

The 96-well plates with the molecular beacons were produced as follows. Molecular beacons were purchased from commercial sources with either a FAM, HEX, ATTO Rho101, or Cy5 fluorophore. For FAM and HEX, the quencher was Black Hole Quencher 1 (BHQ-1), and for ATTO Rho101 and Cy5, the quencher was Black Hole Quencher 2 (BHQ-2). The lyophilized molecular beacons were resuspended in Tris-EDTA as 100 μM stock solutions. The molecular beacon stock solutions for four different beacons were combined into a working stock solution in Tris-EDTA buffer, where each molecular beacon had a concentration of 1.2 μM. For example, a molecular beacon containing a FAM fluorophore and a Serratia spp. target sequence, a molecular beacon containing a HEX fluorophore and a vanA/vanB antibiotic resistance gene target sequence, a molecular beacon containing an ATTO Rho101 fluorophore and a E. faecium target sequence, and a molecular beacon containing a Cy5 fluorophore and a S. pneumoniae target sequence were combined in the molecular beacon working stock solution. 5 μL of the molecular beacon working stock solution was pipetted into each well of a clear Brooks 96-well PCR plate. Plates were prepared prior to experiments and stored at 2-8° C. up to 4 hours.

A two-stage symmetric-asymmetric method is outlined in FIG. 2. Samples were processed as above but amplified for the first 8 cycles with a symmetric multiplex reaction buffer containing 100 nM of each primer. The 96-well plates were prepared using working stocks containing 1.2 μM of each molecular beacons and 1.2 μM of each excess primer. For example, a molecular beacon containing a FAM fluorophore and a Serratia spp. target sequence, a Serratia spp. reverse primer, a molecular beacon containing a HEX fluorophore and a vanA/vanB antibiotic resistance gene target sequence, a vanA/vanB forward primer, a molecular beacon containing an ATTO Rho101 fluorophore and a E. faecium target sequence, a E. faecium forward primer, a molecular beacon containing a Cy5 fluorophore and a S. pneumoniae target sequence, and a S. pneumoniae forward primer in the working stock solution. 5 μL of the molecular beacon and excess primer working stock solution was pipetted into each well of a clear Brooks 96-well PCR plate. Plates were prepared prior to experiments and stored at 2-8° C. up to 4 hours.

### Results

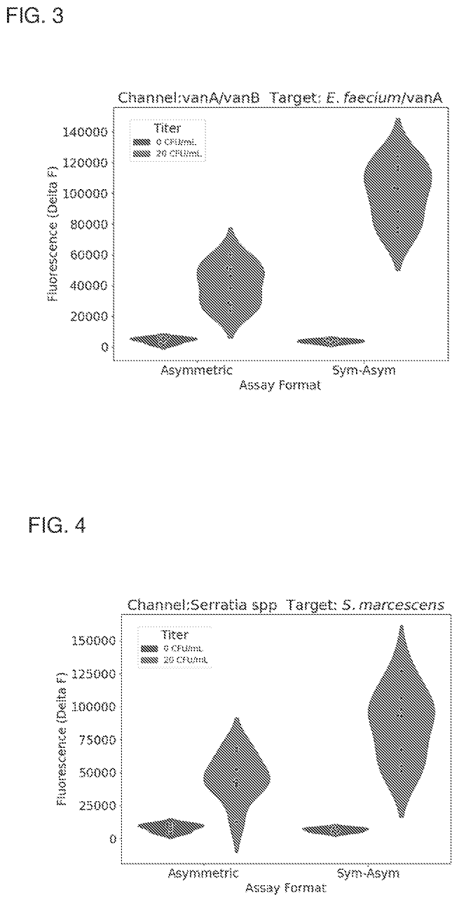

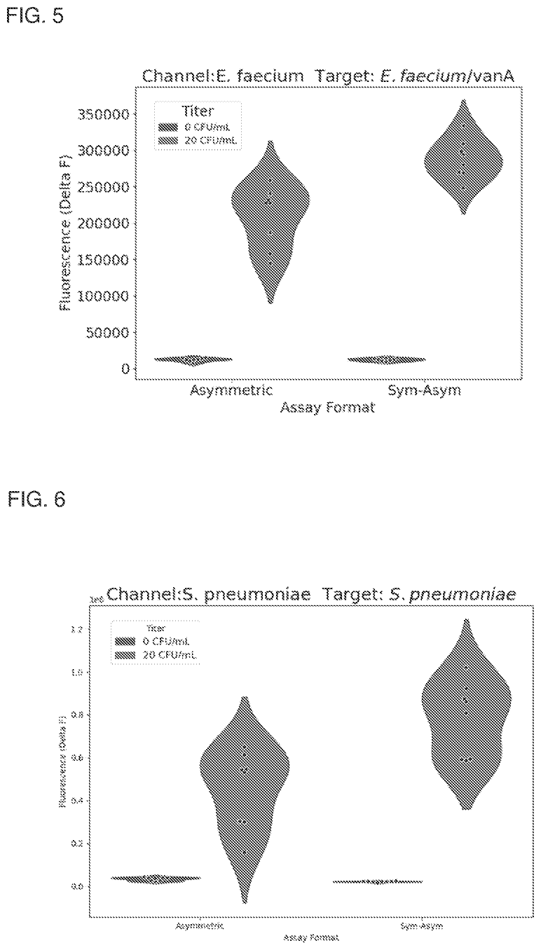

A Serratia marcescens isolate was tested against the Serratia spp. channel, an Enterococcus faecium isolate that contained the vanA antimicrobial resistance gene was tested against both the E. faecium and the vanA/vanB channels, and a Streptococcus pneumoniae isolate was tested against the S. pneumoniae channel using the asymmetric and symmetric-asymmetric methods. Separation between the positive and negatives was observed between all targets and methods (FIGS. 3, 4, 5, and 6). The symmetric-asymmetric method had a higher signal to noise ratio than the asymmetric method for all tested targets.

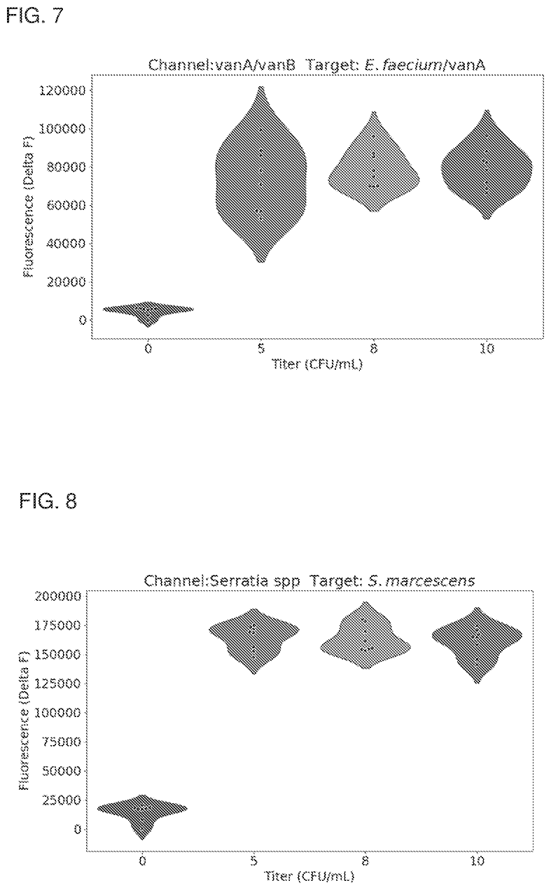

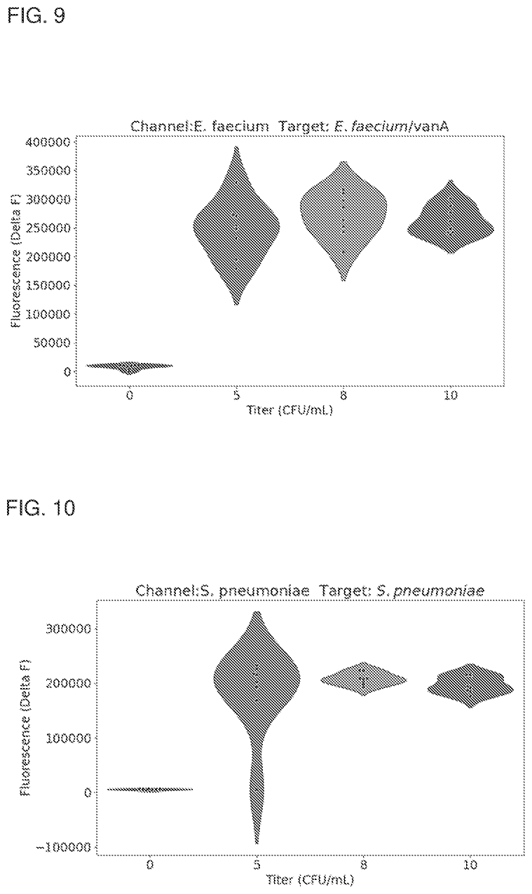

The limit of detection (LoD) was assessed for the channels Serratia spp., E. faecium, vanA/vanB, and S. pneumoniae using the symmetric-asymmetric assay format in which amplification was carried out with 16 PCR cycles in the first stage that was followed by 38 cycles in the second stage. Samples were prepared containing 0, 5, 8, or 10 CFU/mL of isolates of S. marcescens, E. faecium with vanA antimicrobial resistance gene, or S. pneumoniae. Separation of positive and negatives were observed for S. marcescens, E. faecium, and vanA at all tested titers, indicating that the LoD was 5 CFU/mL or less for these targets (FIGS. 7, 8, and 9, respectively). A single false negative was observed at 5 CFU/mL for the S. pneumoniae channel, which suggested that the LoD was between 5 and 10 CFU/mL (FIG. 10).

### Example 2: Cross-Reactivity and Single-Versus Multi-Spike Performance for a Large Multiplex Reaction

A symmetric-asymmetric PCR method was used to assess cross-reactivity and single-versus multi-spike performance for a large resistance gene multiplex reaction. Cross-reactivity was tested with isolates carrying CMY, a combination of OXA-48 and CTX-M, and VIM antibiotic resistance genes. Single versus multi-sample equivalence was tested by comparing individual strains or a mix of all strains containing the CMY, OXA-48, and VIM antibiotic resistance genes. Samples were processed and amplified as described in Example 1 for a symmetric-asymmetric PCR method.

The molecular beacon plate contained 4 separate mixes of molecular beacons and primers. Mix 1 contained molecular beacons and excess primers for the IMP, OXA-48, and VIM antibiotic resistance genes, Mix 2 contained molecular beacons and excess primers for the DHA, KPC, and CTX-M antibiotic resistance genes, Mix 3 contained molecular beacons and excess primers for the FOX and NDM antibiotic resistance genes and the Enterobacterales family target sequence, and Mix 4 contained molecular beacons and excess primers for the CMY, mcr-1, and OXA-23 antibiotic resistance genes. Cross-reactivity within the multiplex targets was assessed by testing a single spike against all multiplex members.

### Results

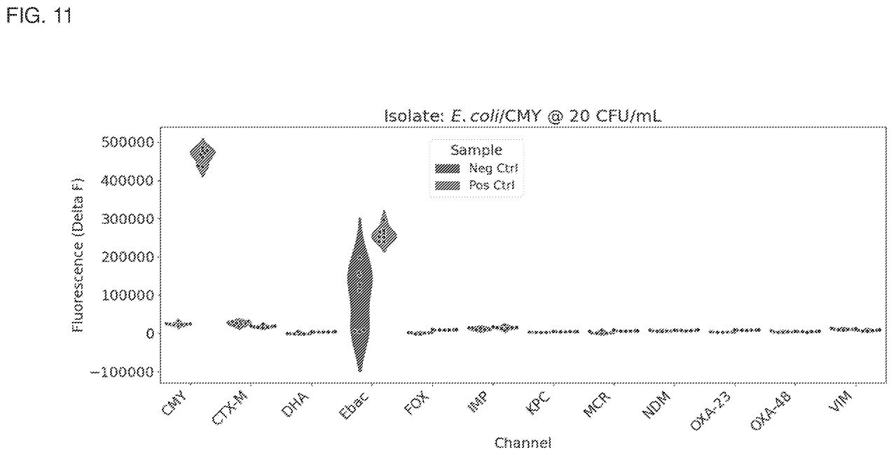

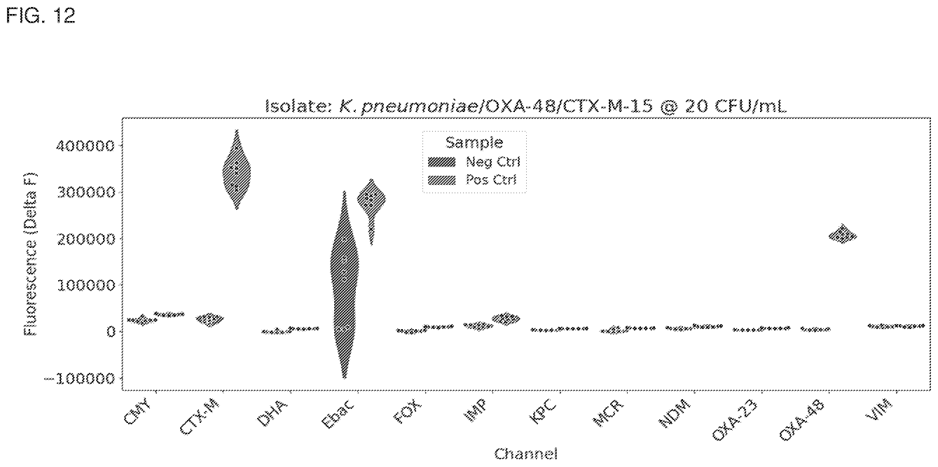

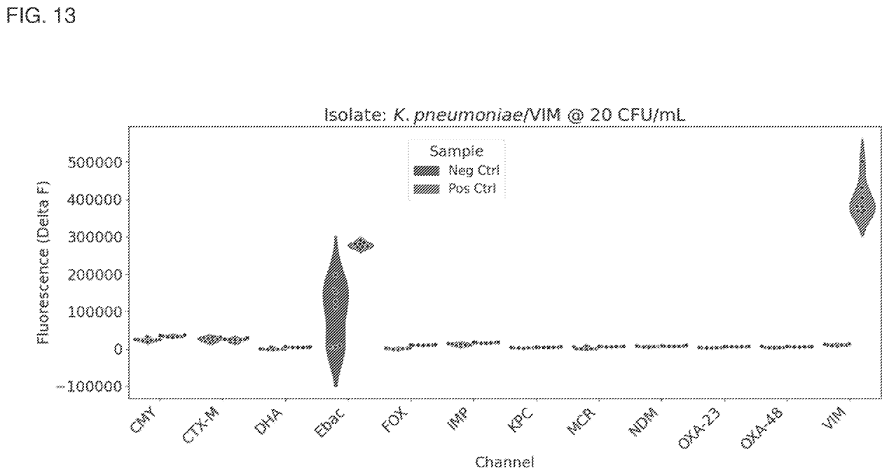

The panel detected that the E. coli isolate containing the CMY antibiotic resistance gene was positively detected for Enterobacterales family and CMY as expected (FIG. 11). A K. pneumoniae isolate carrying both OXA-48 and CTX-M-15 resistance genes was positively detected with the OXA-48, CTX-M, and Enterobacterales channels as expected (FIG. 12). The VIM and Enterobacterales channels were positive for a K. pneumoniae isolate carrying the VIM antibiotic resistance gene, as expected (FIG. 13). No cross-reactivity was observed for any of the isolates with the other channels in the multiplex. Some detection in the Enterobacterales channel was observed in the negative samples, but this can be attributed to potential contamination during amplification.

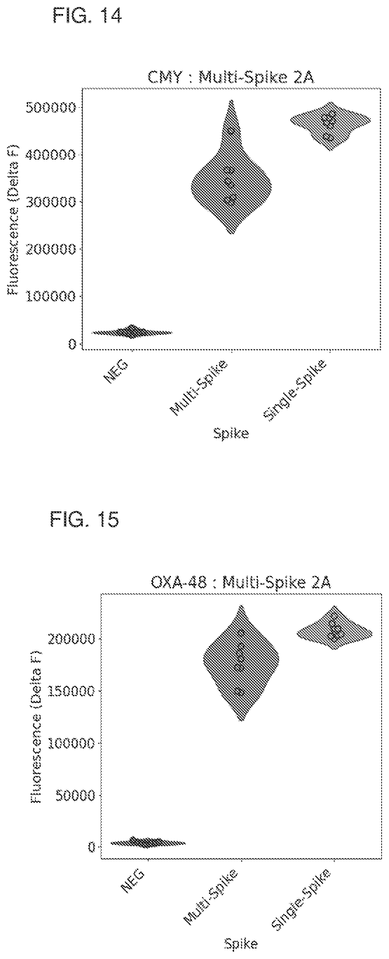

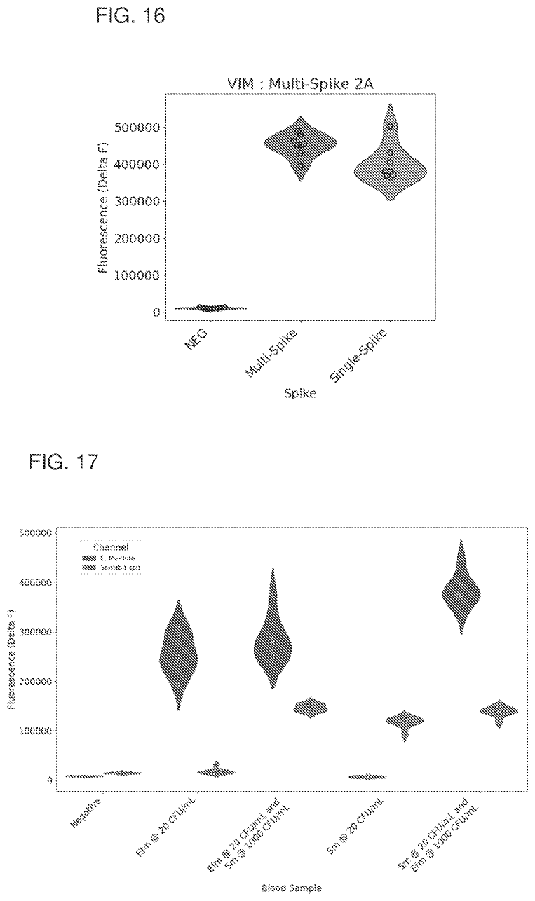

Single- and multi-spike equivalence were determined for the CMY, OXA-48, and VIM channels (FIGS. 14, 15, and 16, respectively). All three tests were statistically equivalent as determined by a two-one sided test using an equivalence limit of 40% and (1−2α)×100% confidence intervals.

### Example 3: Competitive Inhibition

To test whether high titers of one target were inhibitory to the detection of another target in the same multiplex reaction, samples were prepared containing 20 CFU/mL E. faecium, 20 CFU/mL S. marcescens, or combinations of E. faecium and S. marcescens with one target at 20 CFU/mL and the other at 1000 CFU/mL. The samples were processed using the previously described method in Example 1 and amplified using a symmetric-asymmetric PCR method.

### Results

Both E. faecium and S. marcescens were detected in the presence of their corresponding competitors (FIG. 17). The comparison of signal in the presence and absence of inhibitor were determined to be equivalent by a two-one sided t-test with α=0.05, Θ=40%, and (1−2α)×100% confidence intervals.

### Example 4: Two-Stage PCR Permits Higher Sensitivity Across Patients

To assess how the two-stage amplification and detection method and deltaF measurement disclosed herein permits higher sensitivity across patients, the healthy donor and unhealthy patient clinical data were re-analyzed to compare sensitivity between deltaF (two-stage PCR) and step 2 only (one-stage PCR) methods (assessing final fluorescence after 54 cycles for both methods). The cutoff was set for 100% specificity.

### Two-Stage Method

For the two-stage PCR method, a lysate was prepared. Primers and a thermostable DNA polymerase were added to the lysate. PCR was run in the tube for 8 cycles. 15 μL of the reaction product was aliquoted into a multi-well plate, e.g., a 4-well or 96-well plate, containing molecular beacons. 10 μL of mineral oil was added and fluorescence was measured (“stage 1”).

The reaction product was amplified for 46 more cycles in the multi-well plate and fluorescence was measured again (“stage 2”).

Fluorescence at stage 2 minus fluorescence at stage 1 is the response (deltaF).

### One-Stage Method

For the one-stage PCR method, a lysate was prepared. Primers and a thermostable DNA polymerase were added to the lysate. 15 μL of the reaction product was aliquoted into a well on a multi-well plate, e.g., a 96-well plate, containing molecular beacons. 10 μL of mineral oil was added, and fluorescence was measured (“baseline”).

PCR was run for 54 cycles in the multi-well plate and fluorescence is measured (“end”).

Fluorescence at end minus fluorescence at baseline is the response (one-stage).

### Results

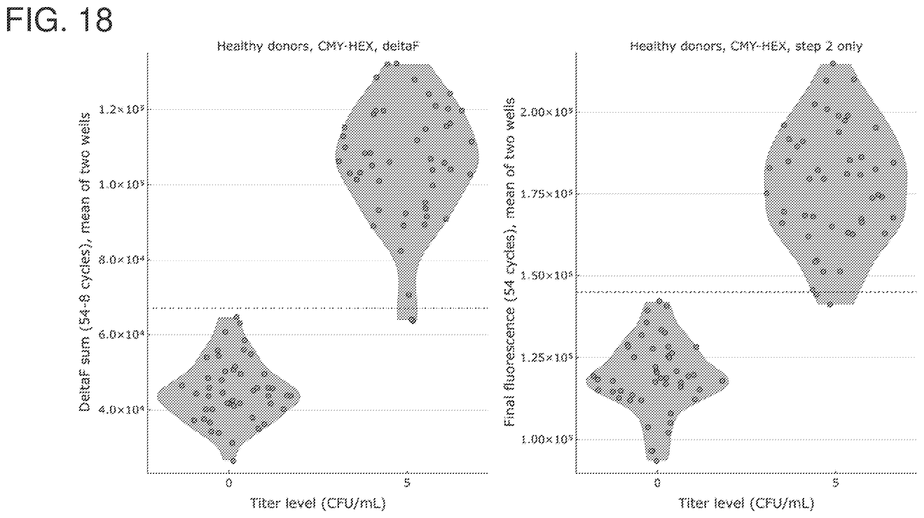

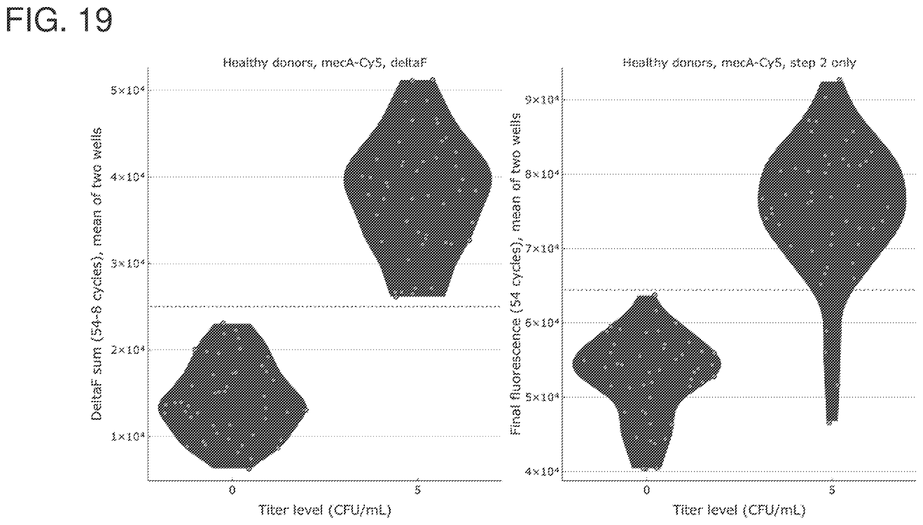

FIG. 18 shows a comparison of the deltaF (two-stage PCR) versus one-stage approach for MRN healthy donors for CMY-HEX. With healthy donors and the CMY target, deltaF (two-stage PCR) and one-stage PCR fluorescence produced the same sensitivity at 5 CFU/mL at 95.8% (46/48). FIG. 19 shows a comparison of the deltaF (two-stage PCR) versus one-stage PCR approach for MRN healthy donors for mecA-Cy5. With healthy donors and the mecA target, deltaF (two-stage PCR) produced 100% sensitivity (48/48), whereas one-stage PCR produced a sensitivity of 91.7% (44/48)

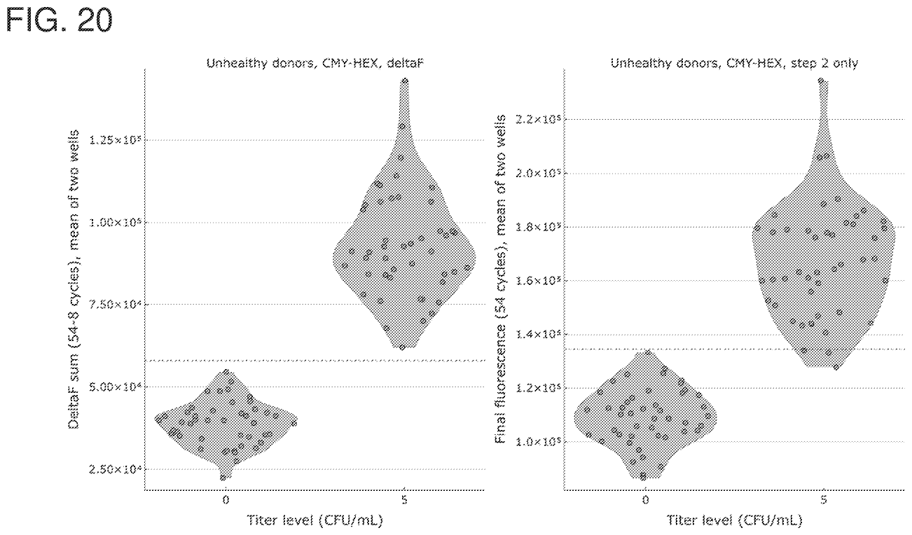

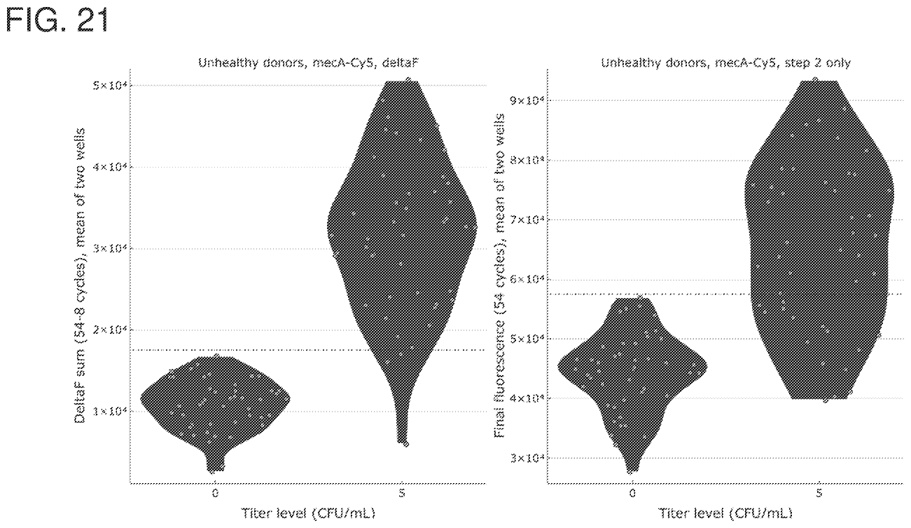

FIG. 20 shows a comparison of the deltaF (two-stage PCR) versus one-stage PCR approach for unhealthy patients for CMY-HEX. With unhealthy donors and the CMY target, the deltaF (two-stage PCR) method produced 100% sensitivity (46/46), whereas the one-stage PCR method produced 93.5% sensitivity (43/46). FIG. 21 shows a comparison of the deltaF (two-stage PCR) versus one-stage PCR approach for unhealthy patients for mecA-Cy5. With unhealthy donors and the mecA target, the deltaF (two-stage PCR) method produced 93.5% sensitivity (43/46), whereas the one-stage PCR method produced 67.4% sensitivity (31/46)

Overall, in healthy and unhealthy donors, at 5 CFU/mL titer the deltaF (two-stage PCR) method provides 10.1% higher sensitivity than the one-stage PCR fluorescence measurement alone (p=0.0003) (Table 4). DeltaF (two-stage PCR) appears to especially benefit sensitivity in hospitalized patients over healthy donors.

TABLE 4
Summary of deltaF (two-stage PCR) vs. one-stage PCR Analysis

| | | | | | | | All data |
|---|---|---|---|---|---|---|---|
| | | | | | | | p-value, |
| | | | | Aggregated | | All data | Fisher's |
| | | | Sensitivity @ | sensitivity @ | p-value, | aggregated | exact test, |
| | | | 5 CFU/mL | 5 CFU/mL | Fisher's | sensitivity @ | deltaF vs. |
| Sample type | Method | Channel | (%) | (%) | exact test | 5 CFU/mL | Step 2 |
| Healthy | deltaF | cmy-hex | 95.8 (46/48) | 97.9 (94/96) | 0.279 | deltaF: 97.3 | 0.0003 |
| donors | | meca-cy5 | 100 (48/48) | | | (183/188) | |
| | Step2 | cmy-hex | 95.8 (46/48) | 93.8 (90/96) | | | |
| | | meca-cy5 | 91.7 (44/48) | | | | |
| Hospitalized | deltaF | cmy-hex | 100 (46/46) | 96.7 (89/92) | 0.0008 | Step2: 87.2 | |
| patients with | | meca-cy5 | 93.5 (43/46) | | | (164/188) | |
| blood culture | Step2 | cmy-hex | 93.5 (43/46) | 80.4 (74/92) | | | |
| within 14 | | meca-cy5 | 67.4 (31/46) | | | | |
| days | | | | | | | |

### Example 5: Additional Data for Asymmetric to Symmetric 2-Stage PCR

Additional experiments were performed to assess whether using a symmetric reaction in the first PCR stage and adding the excess primers specifically needed for each second stage reaction improves fluorescence and signal to noise ratio (SNR).

### Method

The S. agalactiae and E. coli channels of a 12-member multiplex PCR reaction were tested with (i) the two stage method in which both the first and second stages are asymmetric (referred to as “asymmetric to asymmetric”), and (ii) the symmetric to asymmetric method. Genomic DNA was spiked into negative lysate at 25 copies per reaction for each method. The primer concentrations for the symmetric to asymmetric condition in the stage 1 PCR were 100 nM for each forward and reverse primer. In contrast, the asymmetric to asymmetric stage 1 PCR contained 400 nM of each excess primer. For the second stage of PCR, the first stage was split into four smaller reactions in an optical plate for both conditions. The asymmetric to asymmetric plate contained molecular beacons only, while the symmetric to asymmetric plate contained molecular beacons as well as 300 nM of the excess primers to sum to 400 nM for the S. agalactiae and E. coli channels only. Fluorescence measurements were performed as described above, with reads before and after stage 2 PCR to calculate the change in fluorescence, or delta F.

### Results

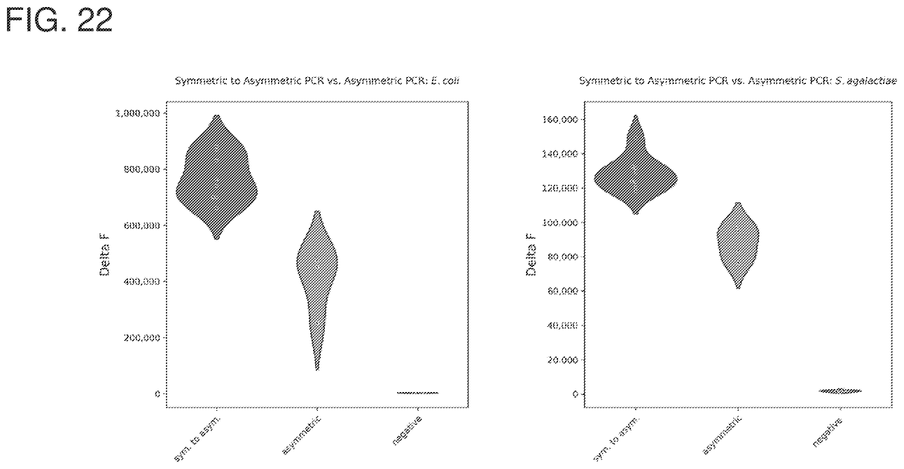

Strikingly, the average delta F for the S. agalactiae channel increased by 46% using the symmetric to asymmetric method (FIG. 22), which was found statistically significant by two-sample T-test (P-value=0.000). Similarly, the average delta F increased by 85% for the E. coli channel (P-value=0.005). SNR also increased from 175 to 258 for S. agalactiae and from 575 to 1068 for E. coli.

Both delta F and SNR increased using the symmetric to asymmetric method of amplification over the asymmetric to asymmetric method. Without wishing to be bound by theory, it is likely that by reducing the overall concentration of primers in both stages of PCR, there is a concurrent reduction in off-target amplification as well as primer-primer interaction that increases the efficiency of the desired amplification.

### Conclusion

The symmetric to asymmetric method significantly improved delta F and SNR for the S. agalactiae and E. coli channels using a 12-member multiplex. The method may be applied to other channels and multiplexes to improve their performance.

### Example 6: Comparison of 1-Stage Amplification Versus 2-Stage Symmetric-Asymmetric Amplification

This example describes a comparison of 1-stage PCR amplification versus a 2-stage symmetric-asymmetric PCR amplification approach as described herein.

### Methods

Whole blood samples spiked with 10 CFU/mL Staphylococcus lugdunensis and Bacillus fragilis were processed using manual assays as described above. The blood lysate was combined with multiplex reaction buffer and denatured at 95° C. for 5 min. The samples were centrifuged for 5 min at 8000×g and formulated thermostable DNA polymerase enzyme was added prior to amplification.

For samples undergoing the 1-stage method, the 100 μL reaction was amplified for 54 cycles, after which 15 μL of amplicon was added to a 96 well plate containing molecular beacons. A single fluorescence read was taken of the 1-stage reaction.

The 2-stage reactions consisted of 16 cycles for the 100 μL reaction followed by the addition of 15 μL of amplicon to a 96 well plate containing molecular beacons. An initial fluorescence read was taken and a second round of amplification was carried out for 38 cycles. A second fluorescence read was taken and Delta F was obtained by subtracting read 1 from read 2.

Signal to noise ratios were calculated as

(Mean positive signal−Mean negative signal)/Standard deviation of Negatives.

### Results

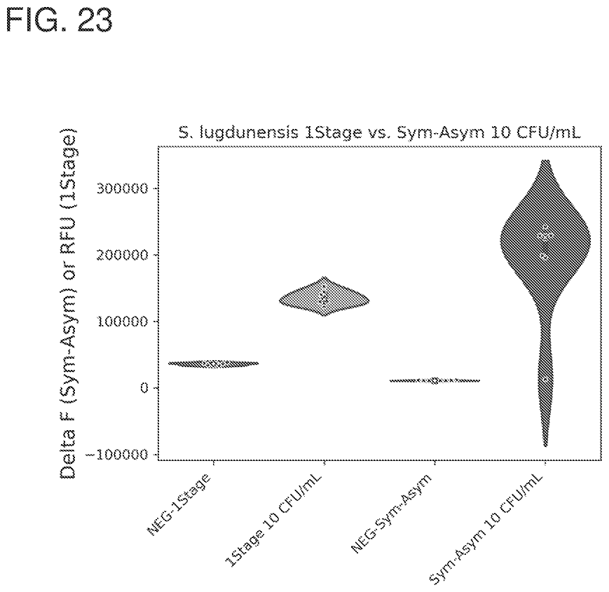

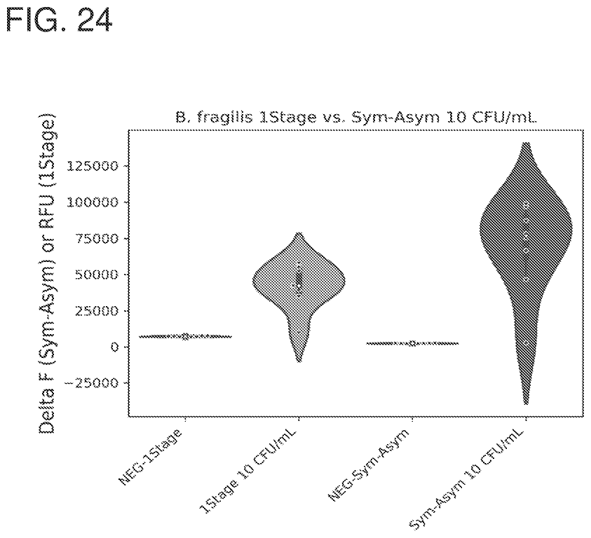

The signal to noise ratios were higher for both S. lugdunensis and B. fragilis using the two stage method (Table 5). The 1-stage method had higher sensitivity than the 2-stage method for S. lugdunensis, as a single false negative was observed in the 2-stage symmetric-asymmetric samples (FIG. 23). The sensitivity was similar for B. fragilis as both methods had a single false negative (FIG. 24).

TABLE 5
Signal to noise ratios for 1-stage and 2-stage methods

| | | 2-stage |
|---|---|---|
| | | symmetric- |
| | 1-stage | asymmetric |
| S. lugdunensis | 51 | 280 |
| B. fragilis | 104 | 302 |

### Conclusion

Higher positive signals and stronger signal to noise ratios were obtained using a 2-stage symmetric-asymmetric amplification method as described herein compared to a 1-stage PCR method.

### Example 7: Exemplary Multiplexed Detection of a Panel of Target Nucleic Acids Using 2-Stage PCR

An exemplary two-stage asymmetric PCR method is outlined in FIG. 1. Whole blood samples (e.g., 6 mL) may be lysed as a single volume, e.g., as described in Example 1, centrifuged (e.g., as described above), and 400-2400 μL (e.g., 1200 μL) of TE buffer can be added, followed by brief homogenization, centrifugation, and aspiration of the supernatant. TE buffer (e.g., 200-500 μL, e.g., 300 μL) is added, the tube is vortexed, and then centrifuged to form the lysate used for amplification.

A panel (e.g., as shown in Table 3) may be detected by performing parallel multiplex reactions. For example, for a panel that includes four multiplex reactions, the lysate from the lysis tube is added to each of four wells of a 4-well plate. The 4-well plate may heated to 95° C. (e.g., for 5 min), centrifuged, and cooled to 25° C. Formulated thermostable DNA polymerase enzyme is added.

The 4-well plate is loaded into a device comprising a thermocycler and incubated at 95° C. (e.g., for 5 min. Next, 6-20 cycles of PCR is performed in a first PCR stage. In some examples, the first PCR stage employs symmetric PCR. The primers for symmetric PCR may be present, e.g., in concentrations of from 50 nM to 300 nM. However, in other examples, asymmetric PCR may be performed in the first PCR stage. The concentration of the excess primer for asymmetric PCR may be between, e.g., 100 nM to 1000 nM.

The amplicon (supernatant) is transferred (e.g., in 15 μL volumes) into a 4-well plate containing a mix of molecular beacons (e.g., present in a 5 μL liquid volume in each plate). Each multiplex from stage 1 is dispensed to a separate plate. For example, each well may include 6 fluorescent channels, with one channel being a reference dye not linked to any nucleic acid sequence, which may serve as a fiduciary mark for the presence of a well, and can be used to correct for system-to-system or well-to-well variability. Mineral oil is added on top of each well. More than one probe can be present for a single fluorescent channel (for example, a target may use two or more (e.g., three) probes of the same color to boost inclusivity of detection). For example, 5-15 distinct probes can be included in each well of the 4-well plate. In some examples, the plate may include a slit seal or may lack a seal.

Fluorescence is measured by a detector included within the device. This measurement is the initial fluorescence measurement. In some examples, the detector includes filters for one or more of ATTO 425, FAM, HEX, ROX, ATTO 633, Cy5.5, or ALEXA 750.

The plate is then cycled for 30-50 additional cycles using asymmetric PCR to generate an asymmetric amplicon. The concentration of the excess primer for asymmetric PCR may be between, e.g., 100 nM to 1000 nM. The plate is then cooled down, e.g., to on board temperature (e.g., about 25° C.). Fluorescence is measured again using the same settings as after the initial amplification. This is the final fluorescence measurement.

The response (“Delta F”) is the final fluorescence minus the initial fluorescence within each well.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for amplifying and detecting a target pathogen nucleic acid in a biological sample suspected of containing one or more pathogen cells, the method comprising:
(a) lysing one or more pathogen cells within the biological sample by contacting the biological sample with a lysis agent to form a solution containing both subject cell nucleic acid and pathogen nucleic acid, and optionally:
(i) centrifuging the product of step (a) to form a supernatant and a pellet;
(ii) discarding some or all of the supernatant of step (i) and optionally washing the pellet once;
(iii) centrifuging the product of step (ii) to form a supernatant and a pellet;
(iv) discarding some or all of the supernatant of step (iii) and mixing the pellet of step (iii) with a buffer;
(b) amplifying the pathogen nucleic acid in the solution of step (a) by 6 to 20 cycles of PCR to form an amplified solution, wherein the PCR is symmetric PCR;
(c) adding a pathogen-specific fluorescent probe to the amplified solution;
(d) measuring a signal from the pathogen-specific fluorescent probe in the amplified solution of step (c) to obtain a baseline measurement;
(e) further amplifying the pathogen nucleic acid in the amplified solution of step (c) by 30 to 50 cycles of PCR, wherein the PCR is asymmetric PCR;
(f) measuring the signal from the pathogen-specific fluorescent probe in the amplified solution of step (e); and
(g) obtaining a final signal measurement by subtracting the baseline measurement from step (d) from the signal from step (f), wherein the presence of the target pathogen in the biological sample is detected based on the final signal measurement.

2. The method of claim 1, wherein the primer concentration for the symmetric PCR of step (b) is between 50 nM to 300 nM.

3. The method of claim 1 or 2, wherein the excess primer concentration for the asymmetric PCR of step (e) is between 100 nM to 1000 nM.

4. The method of any one of claims 1-3, wherein the amplifying of step (b) is 8 cycles of PCR or 16 cycles of PCR.

5. The method of any one of claims 1-4, wherein the amplifying of step (e) is 46 cycles of PCR or 38 cycles of PCR.

6. The method of any one of claims 1-5, wherein the biological sample is a whole blood sample.

7. The method of claim 6, wherein the lysis agent of step (a) is an erythrocyte lysis agent.

8. The method of any one of claims 1-7, wherein the amplifying of step (b) and step (e) is in a multiplexed PCR reaction configured to amplify a plurality of target pathogen nucleic acids.

9. The method of any one of claims 1-8, wherein step (c) comprises adding a plurality of different pathogen-specific fluorescent probes to the amplified solution.

10. The method of claim 9, wherein the plurality of pathogen-specific fluorescent probes comprises three or more different pathogen-specific fluorescent probes.

11. The method of claim 10, wherein the plurality of pathogen-specific fluorescent probes is between 5 and 15 probes.

12. The method of any one of claims 9-11, wherein a plurality of pathogen-specific fluorescent probes is used to detect a single target nucleic acid.

13. The method of any one of claims 1-12, wherein step (c) comprises transferring all or part of the amplified solution of step (b) to a multi-well plate containing the pathogen-specific fluorescent probe.

14. The method of claim 13, wherein the multi-well plate is a 4-well plate, a 96-well plate, or a 384-well plate.

15. The method of claim 14, wherein the multi-well plate is a 4-well plate.

16. The method of any one of claims 13-15, wherein the multi-well plate is a clear plate.

17. The method of any one of claims 13-15, wherein the multi-well plate is a black plate.

18. The method of any one of claims 1-17, wherein the pathogen-specific fluorescent probe of step (c) is present in a liquid volume or is a lyophilized fluorescent probe.

19. The method of any one of claims 13-18, wherein the wells of the multi-well plate each comprise a reference dye.

20. The method of claim 19, wherein the wells of the multi-well plate each comprise the same reference dye.

21. The method of claim 19, wherein the wells of the multi-well plate each comprise different reference dyes.

22. The method of any one of claims 1-21, wherein the pathogen-specific fluorescent probe is a molecular beacon.

23. The method of claim 22, wherein the molecular beacon comprises an organic dye fluorophore.

24. The method of claim 23, wherein the organic dye fluorophore is ATTO 425, FAM, HEX, Cy5, ROX, ATTO 633, TAMARA, Cy5.5, or ALEXA® 750.

25. The method of claim 24, wherein the organic dye fluorophore is ATTO 425, FAM, HEX, ROX, ATTO 633, Cy5.5, or ALEXA® 750.

26. The method of claim 22, wherein the molecular beacon comprises a quantum dot.

27. The method of claim 22, wherein the molecular beacon comprises an organic dye quencher.

28. The method of claim 27, wherein the organic dye quencher is Black Hole Quencher 1, Black Hole Quencher 2, Iowa Black FQ, or Iowa Black RQ.

29. The method of claim 22, wherein the molecular beacon comprises a gold nanoparticle or silica nanoparticle quencher.

30. The method of any one of claims 13-29, wherein an oil is added to a well of the multi-well plate after the amplified solution of step (b) is added.

31. The method of claim 30, wherein the oil is a mineral oil.

32. The method of claim 30, wherein the oil is a silicone oil.

33. The method of any one of claims 13-32, wherein the multi-well plate is sealed with a plastic seal.

34. The method of claim 33, wherein the plastic seal covers the entire multi-well plate.

35. The method of claim 33, wherein the plastic seal is a slit-seal.

36. The method of any one of claims 1-35, wherein the method comprises steps (i)-(iv), and wherein the pellet of step (ii) is washed by mixing with Tris-EDTA (TE) buffer.

37. The method of claim 36, wherein the TE buffer has a volume of about 400 μL to about 2400 μL.

38. The method of claim 37, wherein the TE buffer has a volume of about 1200 μL.

39. The method of any one of claims 36-38, wherein the buffer of step (iv) is a PCR buffer that has a moderately alkaline pH at ambient temperature.

40. The method of any one of claims 1-39, wherein the lysing of step (a) is by detergent lysis, hypotonic lysis, and/or beadbeating.

41. The method of any one of claims 6-40, wherein the amplified solution of step (b) comprises whole blood proteins and non-target oligonucleotides.

42. The method of claim any one of claims 6-41, wherein the whole blood sample is from 0.05 to 10.0 mL.

43. The method of claim 42, wherein the whole blood sample is (i) between about 1.25 and about 2.5 mL; or (ii) about 6 mL.

44. The method of claim 43, wherein the whole blood sample is about 6 mL.

45. A method for amplifying and detecting a target pathogen nucleic acid in a biological sample, the method comprising:
(a) contacting a biological sample suspected of containing one or more pathogen cells with a lysis agent, thereby lysing cells within the biological sample;
(b) centrifuging the product of step (a) to form a supernatant and a pellet;
(c) discarding some or all of the supernatant of step (b) and washing the pellet once;
(d) centrifuging the product of step (c) to form a supernatant and a pellet;
(e) discarding some or all of the supernatant of step (d) and mixing the pellet of (d) with a buffer to form a solution containing both subject cell nucleic acid and pathogen nucleic acid;
(f) amplifying the pathogen nucleic acid in the solution of step (e) by 6 to 20 cycles of polymerase chain reaction (PCR) to form an amplified solution;
(g) adding a pathogen-specific fluorescent probe to the amplified solution;
(h) measuring a signal from the pathogen-specific fluorescent probe in the amplified solution of step (g) to obtain a baseline measurement;
(i) further amplifying the pathogen nucleic acid in the amplified solution of step (g) by 30 to 50 cycles of PCR;
(j) measuring the signal from the pathogen-specific fluorescent probe in the amplified solution of step (i); and
(k) obtaining a final signal measurement by subtracting the baseline measurement from step (h) from the signal from step (j), wherein the presence of the target pathogen in the biological sample is detected based on the final signal measurement.

46. The method of claim 45, wherein the amplifying of step (f) is symmetric PCR or asymmetric PCR.

47. The method of claim 46, wherein the amplifying of step (f) is symmetric PCR.

48. The method of claim 47, wherein the primer concentration for the symmetric PCR is between 50 nM to 300 nM.

49. The method of claim 46, wherein the amplifying of step (f) is asymmetric PCR.

50. The method of any one of claims 45-49, wherein the amplifying of step (i) is asymmetric PCR.

51. The method of claim 49 or 50, wherein the excess primer concentration for the asymmetric PCR is between 100 nM to 1000 nM.

52. The method of any one of claims 45-51, wherein the amplifying of step (f) is 8 cycles of PCR or 16 cycles of PCR.

53. The method of any one of claims 45-52, wherein the amplifying of step (i) is 46 cycles of PCR or 38 cycles of PCR.

54. The method of any one of claims 45-53, wherein the biological sample is a whole blood sample.

55. The method of claim 54, wherein the lysis agent of step (a) is an erythrocyte lysis agent.

56. The method of any one of claims 45-55, wherein the amplifying of step (f) and step (i) is in a multiplexed PCR reaction configured to amplify a plurality of target pathogen nucleic acids.

57. The method of any one of claims 45-56, wherein step (g) comprises adding a plurality of different pathogen-specific fluorescent probes to the amplified solution.

58. The method of claim 57, wherein the plurality of pathogen-specific fluorescent probes comprises three or more different pathogen-specific fluorescent probes.

59. The method of claim 58, the plurality of pathogen-specific fluorescent probes is between 5 and 15 probes.

60. The method of any one of claims 57-59, wherein a plurality of pathogen-specific fluorescent probes is used to detect a single target nucleic acid.

61. The method of any one of claims 45-60, wherein step (g) comprises transferring all or part of the amplified solution of step (f) to a multi-well plate containing the pathogen-specific fluorescent probe.

62. The method of claim 61, wherein the multi-well plate is a 4-well plate, a 96-well plate, or a 384-well plate.

63. The method of claim 62, wherein the multi-well plate is a 4-well plate.

64. The method of any one of claims 61-63, wherein the multi-well plate is a clear plate.

65. The method of any one of claims 61-63, wherein the multi-well plate is a black plate.

66. The method of any one of claims 45-65, wherein the pathogen-specific fluorescent probe of step (f) is present in a liquid volume or is a lyophilized fluorescent probe.

67. The method of any one of claims 61-66, wherein the wells of the multi-well plate each comprise a reference dye.

68. The method of claim 67, wherein the wells of the multi-well plate each comprise the same reference dye.

69. The method of claim 67, wherein the wells of the multi-well plate each comprise different reference dyes.

70. The method of any one of claims 45-69, wherein the pathogen-specific fluorescent probe is a molecular beacon.

71. The method of claim 70, wherein the molecular beacon comprises an organic dye fluorophore.

72. The method of claim 71, wherein the organic dye fluorophore is ATTO 425, FAM, HEX, Cy5, ROX, ATTO 633, TAMARA, Cy5.5, or ALEXA® 750.

73. The method of claim 72, wherein the organic dye fluorophore is ATTO 425, FAM, HEX, ROX, ATTO 633, Cy5.5, or ALEXA® 750.

74. The method of claim 70, wherein the molecular beacon comprises a quantum dot.

75. The method of claim 70, wherein the molecular beacon comprises an organic dye quencher.

76. The method of claim 75, wherein the organic dye quencher is Black Hole Quencher 1, Black Hole Quencher 2, Iowa Black FQ, or Iowa Black RQ.

77. The method of claim 70, wherein the molecular beacon comprises a gold nanoparticle or silica nanoparticle quencher.

78. The method of any one of claims 1-77, wherein an oil is added to a well of the multi-well plate after the amplified solution of step (f) is added.

79. The method of claim 78, wherein the oil is a mineral oil.

80. The method of claim 78, wherein the oil is a silicone oil.

81. The method of any one of claims 61-80, wherein the multi-well plate is sealed with a plastic seal.

82. The method of claim 81, wherein the plastic seal covers the entire multi-well plate.

83. The method of claim 81, wherein the plastic seal is a slit-seal.

84. The method of any one of claims 1-83, wherein the pellet of step (c) is washed by mixing with Tris-EDTA (TE) buffer.

85. The method of claim 84, wherein the TE buffer has a volume of about 400 μL to about 2400 μL.

86. The method of claim 85, wherein the TE buffer has a volume of about 1200 μL.

87. The method of any one of claims 45-86, wherein the buffer of step (e) is a PCR buffer that has a moderately alkaline pH at ambient temperature.

88. The method of any one of claims 1-87, wherein the lysing step (a) is by detergent lysis, hypotonic lysis, and/or beadbeating.

89. The method of any one of claims 54-88, wherein the amplified solution of step (f) comprises whole blood proteins and non-target oligonucleotides.

90. The method of claim any one of claims 54-89, wherein the whole blood sample is from 0.05 to 10.0 mL.

91. The method of claim 90, wherein the whole blood sample is (i) between about 1.25 and about 2.5 mL; or (ii) about 6 mL.

92. The method of claim 91, wherein the whole blood sample is about 6 mL.

93. The method of any one of claims 1-92, wherein the pathogen is a Candida species.

94. The method of claim 93, wherein the Candida species is selected from the group consisting of Candida albicans, Candida krusei, Candida glabrata, Candida parapsilosis, Candida auris, Candida lusitaniae, Candida dubliniensis, Candida kefyr, and Candida tropicalis.

95. The method of any one of claims 1-44 and 94, wherein the amplifying of steps (b) and (e) comprise amplifying a Candida nucleic acid to be detected in the presence of a forward primer and a reverse primer, each of which is universal to multiple Candida species to form a solution comprising a Candida amplicon.

96. The method of any one of claims 45-94, wherein the amplifying of steps (f) and (i) comprise amplifying a Candida nucleic acid to be detected in the presence of a forward primer and a reverse primer, each of which is universal to multiple Candida species to form a solution comprising a Candida amplicon.

97. The method of any one of claims 1-92, wherein the pathogen is a bacterial pathogen.

98. The method of claim 97, wherein the bacterial pathogen is selected from the group consisting of Bacteroides fragilis, Burkholderia cepacia, Campylobacter jejuni/coli, Clostridium perfringens, Klebsiella aerogenes, Enterobacter cloacae, Enterococcus faecalis, Enterococcus faecium, Escherichia coli, Haemophilus influenzae, Kingella kingae, Klebsiella oxytoca, Klebsiella pneumoniae, Listeria monocytogenes, Morganella morganii, Neisseria meningitidis, Prevotella buccae, Prevotella intermedia, Prevotella melaninogenica, Propionibacterium acnes, Proteus mirabilis, Proteus vulgaris, Pseudomonas aeruginosa, Salmonella enterica, Serratia marcescens, Staphylococcus aureus, Staphylococcus haemolyticus, Staphylococcus lugdunensis, Stenotrophomonas maltophilia, Staphylococcus saprophyticus, Streptococcus agalactiae, Streptococcus bovis, Streptococcus dysgalactiae, Streptococcus mitis, Streptococcus mutans, Streptococcus pneumoniae, Streptococcus pyogenes, and Streptococcus sanguinis.

99. The method of claim 98, wherein the bacterium is selected from the group consisting of Enterococcus faecalis, Enterococcus faecium, Staphylococcus aureus, Klebsiella pneumoniae, Acinetobacter baumannii, and Pseudomonas aeruginosa.

100. The method of claim 99, wherein the bacterial pathogen is Escherichia coli.

101. The method of claim 97, wherein the bacterium is selected from one or more of the group consisting of Escherichia coli, Enterococcus faecium, Staphylococcus aureus, Klebsiella pneumoniae, Acinetobacter baumannii and Pseudomonas aeruginosa.

102. The method of claim 101, wherein the Staphylococcus aureus is methicillin-resistant Staphylococcus aureus (MRSA).

103. The method of claim 97, wherein the bacterial pathogen is a Borrelia species.

104. The method of claim 103, wherein the Borrelia species is Borrelia burgdorferi, Borrelia afzelii, or Borrelia garinii.

105. The method of any one of claims 1-92, wherein the target pathogen nucleic acid is characteristic of a genus of bacterial pathogens.

106. The method of claim 105, wherein the genus of bacterial pathogens comprises coagulase negative staphylococci (CoNS), Enterobacter cloacae complex, Acinetobacter baumannii-calcoaceticus complex, Enterobacterales, Streptococcus spp., Citrobacter spp., Proteus spp., Serratia spp., Clostridium spp., Bacteroides spp., Pan Gram negative, or pan Gram positive.

107. The method of any one of claims 1-92, wherein the target pathogen nucleic acid is an antibiotic resistance target nucleic acid.

108. The method of claim 107, wherein the antibiotic resistance target nucleic acid is mecA, mecC, KPC, NDM, VIM, IMP, OXA-48-like, CTX-M, CMY, DHA, FOX, mcr-1, OXA-23-like, or van A/vanB.

109. The method of any one of claims 1-92, wherein the pathogen is a viral pathogen.

110. The method of claim 109, wherein the viral pathogen is a Cytomegalovirus (CMV), an Epstein Barr Virus, a BK Virus, a Hepatitis B virus, a Hepatitis C virus, a Herpes simplex virus (HSV), HSV1, HSV2, a Respiratory syncytial virus (RSV), an Influenza virus, Influenza A virus, Influenza A subtype H1 virus, Influenza A subtype H3 virus, Influenza B virus, Human Herpes Virus 6, Human Herpes Virus 8, Human Metapneumovirus (hMPV), a Rhinovirus, Parainfluenza 1 virus, Parainfluenza 2 virus, Parainfluenza 3 virus, an Adenovirus, or a Coronavirus.

111. The method of any one of claims 1-92, wherein the method comprises detecting the panel set forth in Table 3.

112. The method of any one of claims 1-111, further comprising selecting a therapy for the patient based on the detection of the target pathogen nucleic acid.

113. The method of claim 112, further comprising administering the therapy to the patient based on the detection of the target pathogen nucleic acid.
